(19) United States
(12) Patent Application Publication
(10) Pub. No.: US 2022/0169607 A1
(43) Pub. Date: Jun. 2, 2022

(54) CARBAZOLE AND ACRIDINE PHOTOREDOX CATALYSTS FOR SMALL MOLECULE AND MACROMOLECULAR TRANSFORMATIONS

(71) Applicant: Colorado State University Research Foundation, Fort Collins, CO (US)

(72) Inventors: Bonnie L. Buss, Fort Collins, CO (US); Garret Miyake, Fort Collins, CO (US); Chern-Hooi Lim, Boulder, CO (US); Max Kudisch, Fort Collins, CO (US)

(73) Assignee: Colorado State University Research Foundation, Fort Collins, CO (US)

(21) Appl. No.: 17/593,101

(22) PCT Filed: Mar. 30, 2020
(86) PCT No.: PCT/US2020/025769

§ 371 Date: Sep. 9, 2021

Related U.S. Application Data
(60) Provisional application No. 62/826,633, filed on Mar. 29, 2019.

(51) Int. Cl. C07D 209/86; C07D 219/02; C07D 265/38

(52) U.S. Cl. CPC C07D 209/86; C07D 265/38; C07D 219/02

(57) ABSTRACT

The present invention provides photocatalysts, methods for their preparation, and methods for preparing linear polymers with high propagation rate.

### RELATED APPLICATIONS

This application claims the benefit of the filing date of U.S. Provisional Application No. 62/826,633, filed Mar. 29, 2019, the contents of which are herein incorporated by reference in its entirety.

### GOVERNMENTAL RIGHTS

This invention was made with government support under R35 GM119702 awarded by the National Institutes of Health. The government has certain rights in the inventions disclosed.

### FIELD OF THE INVENTION

The present disclosure generally relates to organic photocatalysts, the methods for their preparation, and methods of preparing non-statistical, linear polymers with high propagation rate constants.

### BACKGROUND OF THE INVENTION

The ability of photocatalysts to manipulate electron or energy transfer reactivity has revolutionized small molecule and macromolecular chemistry, presenting opportunities to develop new chemical transformations under mild and energy efficient reaction conditions. Recently, photoredox catalysis has been applied in controlled radical polymerization (CRP) approaches for light-regulated synthesis of well-defined polymers, most commonly in atom transfer radical polymerization (ATRP) and reversible addition-fragmentation transfer (RAFT). ATRP, the most widely studied CRP methodology, is used to access polymers with controlled properties, higher-order architectures, and consequently diverse applications. Traditionally, ATRP is operated through activation of a Cu(I) catalyst by heat to promote an inner-sphere electron transfer to generate a propagating radical species. However, in recent advances, new light-driven ATRP processes have been reported using photocatalysts (PCs) derived from copper, ruthenium, or iridium.

Organocatalyzed atom transfer radical polymerization (O-ATRP) is a metal-free variant of photoredox-catalyzed ATRP which eliminates the concern of trace metal contamination in the polymer product and is advantageous in electronic and biomedical applications, while also enabling opportunities for “greener” reaction design in polymer synthesis. Induced by light, O-ATRP relies on a strongly reducing organic PC to mediate an oxidative quenching catalytic cycle (FIG. 1). O-ATRP processes following a reductive quenching pathway have also been reported but rely on the presence of stoichiometric quantities of sacrificial electron donors, which can also induce undesirable side reactions. The proposed O-ATRP mechanism proceeds through four central steps. Photoexcitation of a ground-state PC generates ¹PC*, which can undergo intersystem crossing to produce a long-lived ³PC*. Either ¹PC* or ³PC* then directly reduces an alkyl halide initiator through outer-sphere electron transfer to produce a propagating radical species, as well as the ion-pair ²PC^{●+}X⁻. Deactivation of the propagating chain-end occurs through reinstallation of the halide, generating the PC and a dormant polymer. Central to success in O-ATRP, as determined by control over polymer molecular weight (MW) and dispersity (Ð) close to 1.0, is the presence of a dynamic equilibrium between the activation and deactivation steps, where the rate of deactivation (with rate constant k_{d}) must be higher than the rates of propagation (kₚ) and activation (kₐ), limiting radical concentrations and undesirable termination events via radical quenching.

To date, advances in O-ATRP have been enabled through development of strongly reducing organic PCs, which are capable of directly reducing an activated alkyl bromide ATRP initiator or dormant polymer chain-end (˜−0.8 V vs. SCE). In 2014, perylene and N-phenyl phenothiazine were reported as strongly reducing PCs for the polymerization of methacrylate monomers via O-ATRP. Since then, other organic PCs derived from N,N-diaryl dihydrophenazine and N-aryl phenoxazine families, among others, have been developed. N-aryl phenothiazine PCs have been applied in diverse contexts and have also been used for mechanistic analysis. PC structure-property relationships have been studied using N,N-diaryl dihydrophenazine and N-aryl phenoxazine PCs. Empirically, these studies have determined key PC design principles for effective catalytic performance in O-ATRP, among which are the ability of the PC to exhibit intramolecular charge transfer (CT) excited states, redox reversibility, and sufficient thermodynamic driving forces (redox potentials) to mediate the oxidative quenching O-ATRP cycle. N,N-diaryl Dihydrophenazine and N-aryl phenoxazine PCs have also been studied in diverse polymerization-related contexts, including the effects of light intensity and solvent, adaptation of O-ATRP to continuous-flow reactors, synthesis of star polymers, and demonstration of oxygen tolerance. In addition, these organic PCs were also applied in small molecule reactions, including trifluoromethylation, C—N and C—S cross couplings via dual catalytic approach with Ni(II) salts, and the reduction of carbon dioxide to methane using sunlight for solar fuel generation.

Despite advances in PC design, O-ATRP has largely been limited to polymerization of methacrylate monomers, but the controlled polymerization of other monomers is highly desired. Poly(acrylates) possess disparate thermal and mechanical properties, enabling widespread industrial and academic use, including drug delivery, superabsorbent materials, coatings, adhesives and additive manufacturing. As such, we sought to leverage current understanding of organic PC design to target the O-ATRP of acrylate monomers. The CRP of acrylates is inherently challenging due to high kₚ, with values ranging from 15,000 to 24,000 L mol⁻¹s⁻¹, an order of magnitude larger than methacrylates. Furthermore, acrylate chain-end groups containing bromides are more difficult to reduce compared to the corresponding methacrylates, emphasizing the need for efficient PCs for activation.

Photoinduced copper-catalyzed ATRP processes have been reported for successful CRP of acrylate monomers. Additionally, a photoredox-catalyzed ATRP approach of acrylates was also performed using a precious metal-based fac-Ir(ppy)₃ PC following an oxidative quenching catalytic cycle, accessing control over MW growth to produce polymers with moderate Ð. To access this polymerization with an organic PC, we hypothesized that a PC candidate must possess both a sufficiently oxidizing ²PC^{●+} with a correspondingly high E_{1/2} (²PC^{●+}/¹PC) potential to promote fast deactivation (high k_{d}) to compensate for high kₚ, but also maintain a strongly reducing ³PC* with sufficiently negative E⁰(²PC^{●+}/³PC*) value for efficient alkyl bromide activation.

Using density functional theory (DFT) calculations to guide organic PC design, herein dimethyl-dihydroacridines are reported as a new class of organic PCs adept at controlled polymerizations of acrylate monomers via O-ATRP. Due to structural similarity to previously employed PCs in O-ATRP and tunable donor-acceptor motifs, we sought to investigate this class of molecules for use in photoredox-catalyzed processes, accessing tailored photo- and electrochemical properties. In this approach, well-defined acrylate polymers with controlled molecular weights and low dispersities (Ð<1.20) were synthesized using a 365 nm LED in a continuous-flow reactor in conjunction with LiBr salt additives, which are hypothesized to promote efficient deactivation.

What is needed is a photo redox catalyst that can polymerize various acrylate and methacrylate monomers to form a non-statistical, linear polymer with a high propagation rate.

### FIGURES

The application file contains at least one photograph executed in color. Copies of this patent application publication with color photographs will be provided by the Office upon request and payment of the necessary fee.

FIG. 1 shows the proposed mechanistic cycle for O-ATRP, in accordance with embodiments of the disclosure.

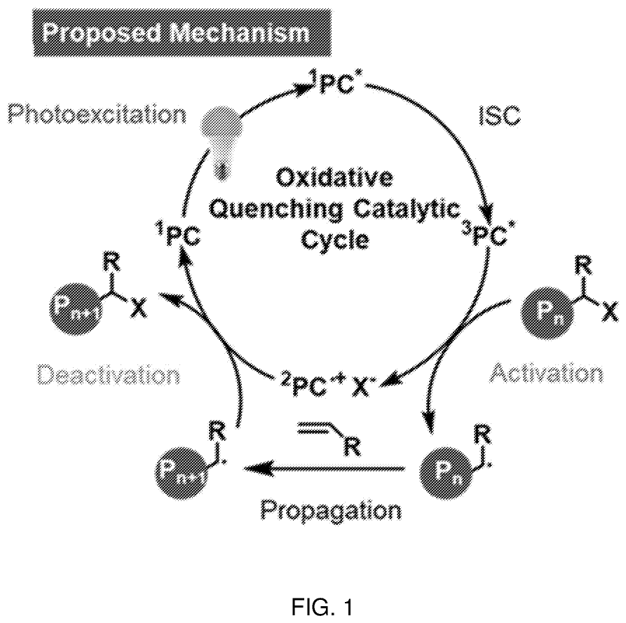

FIG. 2 shows the UV-vis analysis for three exemplary carbazole photoredox catalysts, in accordance with embodiments of the disclosure.

FIG. 3 shows the UV-vis analysis for three exemplary acirdine photoredox catalysts, PC 1, 2 and 3, in accordance with embodiments of the disclosure.

FIG. 4 illustrates an exemplary Reaction Scheme according to an embodiment of the disclosure, in accordance with embodiments of the disclosure.

FIGS. 5A-5B illustrate the solvatochromism of selective carbazole PCs, in accordance with embodiments of the disclosure.

FIGS. 6A-6D illustrated the solvatochromism of selective acridine PCs, in accordance with embodiments of the disclosure.

FIGS. 7A-7C show cyclic voltammetry of exemplary carbazole PCs with noted oxidation potentials, in accordance with embodiments of the disclosure.

FIGS. 8A-8F show the triblock copolymer and the associated data. Plots of MW vs conversion (blue) and Ð vs. conversion (red) for O-ATRP of butyl acrylate without LiBr (A) and with LiBr (D). Corresponding GPC MALS traces are shown in B and E, while corresponding GPC RI traces are shown in C and F.

### SUMMARY OF THE INVENTION

In one aspect, disclosed herein, are compounds of Formula (IV) or salt thereof:

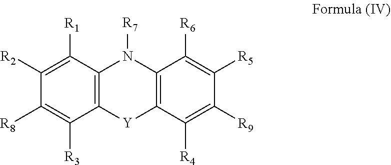

wherein:

R₁, R₂, R₃, R₄, R₅, and R₆ are independently selected from a group consisting of hydrogen, C₁-C₈ substituted or unsubstituted alkyl optionally substituted with at least one heteroatom; C₆-C₂₀ substituted or unsubstituted aryl optionally substituted with at least one heteroatom;

R₇ is selected group a group consisting of C₁-C₈ substituted or unsubstituted alkyl optionally substituted with at least one heteroatom; C₆-C₂₀ substituted or unsubstituted aryl optionally substituted with at least one heteroatom;

R₈ and R₉ are independently selected from a group consisting of C₆-C₂₀ substituted or unsubstituted aryl optionally substituted with at least one heteroatom;

Y is O, CR₁₀R₁₁, or absent; and

R₁₀ and R₁₁ are independently selected from a group consisting of hydrogen, C₁-C₈ substituted or unsubstituted alkyl optionally substituted with at least one heteroatom and C₆-C₁₄ substituted or unsubstituted aryl optionally substituted with at least one heteroatom.

In another aspect, provided herein, are processes to prepare the compound of Formula (IV) or salt thereof:

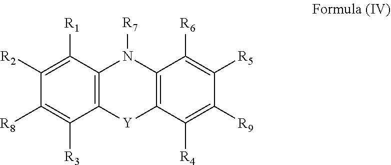

The processes comprising contacting the compound comprising Formula (I):

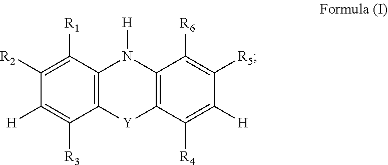

in the presence of an aromatic halide and a catalyst to form a compound comprising Formula (II):

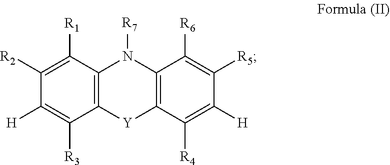

contacting the compound comprising Formula (II) with a halogenation reagent forming the compound comprising Formula (III):

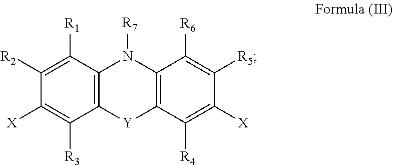

and

contacting the compound comprising Formula (III) with an aryl boronic acid in the presence of a catalyst to prepare the compound comprising Formula (IV).

In yet another aspect, provided herein, are methods for preparing non-statistical, linear polymers. The methods comprising: (a) generating a reaction mixture comprising contacting monomer A, the compound of Formula (IV) or salt thereof, an initiator (In), a bromide source, and a solvent; (b) irradiating the reaction mixture with UV light to generate the linear polymer In-Aₘ-X; (c) isolating at least a portion of linear polymer In-Aₘ-X; (d) generating a second reaction mixture comprising the linear polymer In-Aₘ-X from step (c), monomer B, a bromide source, and a solvent; (e) irradiating the second reaction mixture with UV light to form a linear polymer In-Aₘ-Bₙ—X; and (f) isolating at least a portion of linear polymer In-Aₘ-Bₙ—X wherein n and m are integers ranging from 1 to 10,000 and X is Cl, Br, or I. The methods further comprising preparing a third reaction mixture comprising contacting the linear polymer In-Aₘ-Bₙ—X, monomer C, a bromide source, and a solvent; irradiating the third reaction mixture with UV light to form In-Aₘ-Bₙ—Cₒ—X; and isolating at least a portion of the linear polymer In-Aₘ-Bₙ—Cₒ—X wherein o is an integer from 1 to 10,000 and X is Cl, Br, or I.

Other features and iterations of the invention are described in more detail below.

### DETAILED DESCRIPTION OF THE INVENTION

In one aspect, the present disclosure provides a compound comprising Formula (IV) or a salt thereof. The compound comprising Formula (IV) or a salt thereof are organic photocatalysts (PCs) or organocatalyzed atom transfer radical polymerization (O-ATRP) catalysts useful in acrylate/methacrylate polymer preparation with a high propagation constant.

### (I). Compound Comprising Formula (IV)

In one aspect, the present disclosure provides a compound comprising Formula (IV) or a salt thereof:

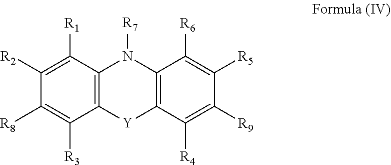

wherein R₁, R₂, R₃, R₄, R₅, and R₆ are independently selected from a group consisting of hydrogen, C₁-C₈ substituted or unsubstituted alkyl optionally substituted with at least one heteroatom; C₆-C₂₀ substituted or unsubstituted aryl optionally substituted with at least one hetero atom; R₇ is selected group a group consisting of C₁-C₈ substituted or unsubstituted alkyl optionally substituted with at least one heteroatom, C₆-C₂₀ substituted or unsubstituted aryl optionally substituted with at least one hetero atom; R₈ and R₉ are independently selected from a group consisting of C₆-C₂₀ substituted or unsubstituted aryl optionally substituted with at least one heteroatom; Y is O, CR₁₀, R₁, or absent; and R₁₀ and R₁₁ are independently selected from a group consisting of hydrogen, C₁-C₈ substituted or unsubstituted alkyl optionally substituted with at least one heteroatom, and C₆-C₁₄ substituted or unsubstituted aryl optionally substituted with at least one heteroatom.

Generally, in accordance with embodiments, R₁, R₂, R₃, R₄, R₅, and R₆ are independently selected from a group consisting of hydrogen, C₁-C₈ substituted or unsubstituted alkyl optionally substituted with at least one heteroatom; C₆-C₂₀ substituted or unsubstituted aryl optionally substituted with at least one hetero atom. In some embodiments, R₁, R₂, R₃, R₄, R₅, and R₆ are independently selected from a group consisting of hydrogen, C₁-C₆ substituted or unsubstituted alkyl optionally substituted with at least one heteroatom; C₆-C₁₄ substituted or unsubstituted aryl optionally substituted with at least one hetero atom. In certain embodiments, R₁, R₂, R₃, R₄, R₅, and R₆ are independently selected from a group consisting of hydrogen, C₁-C₄ substituted or unsubstituted alkyl optionally substituted with at least one heteroatom; C₆-C₁₄ substituted or unsubstituted aryl optionally substituted with at least one hetero atom. In specific embodiments, R₁, R₂, R₃, R₄, R₅, and R₆ are hydrogen.

In general, with accordance with embodiments, R₇ is selected group a group consisting of C₁-C₈ substituted or unsubstituted alkyl optionally substituted with at least one heteroatom; C₆-C₂₀ substituted or unsubstituted aryl optionally substituted with at least one heteroatom. In some embodiments, R₇ is selected from a group consisting of C₁-C₆ substituted or unsubstituted alkyl optionally substituted with at least one heteroatom, C₆-C₁₄ substituted or unsubstituted aryl optionally substituted with at least one heteroatom. In certain embodiments, R₇ is selected from a group consisting of C₁-C₄ substituted or unsubstituted alkyl optionally substituted with at least one heteroatom; C₆-C₁₄ substituted or unsubstituted aryl optionally substituted with at least one hetero atom. In specific embodiments, R₇ is selected from a group consisting of methyl, ethyl, phenyl, 1-naphthyl, 2-naphthyl, phenyl, 4-methoxyphenyl, or 4-cyanophenyl.

Generally, in accordance with embodiments, R₈ and R₉ are independently selected from a group consisting of C₆-C₂₀ substituted or unsubstituted aryl optionally substituted with at least one hetero atom. In certain embodiments, R₈ and R₉ are independently selected from a group consisting of C₆-C₂₀ substituted or unsubstituted aryl optionally substituted with at least one hetero atom. In some embodiments, R₈ and R₉ are independently selected from a group consisting of C₆-C₂₀ substituted or unsubstituted aryl optionally substituted with at least one hetero atom. In specific embodiments, R₈ and R₉ are independently selected from a group consisting of 4, 4′-biphenyl, 4-methoxyphenyl, or 4-cyanophenyl.

Generally, Y is O, CR₁₀R₁₁, or absent. In specific embodiments, Y is O, CR₁₀R₁₁, or absent. In some embodiments, Y is O or absent. In other embodiments, Y is CR₁₀R₁₁. In yet other embodiments, Y is O. In yet other embodiments, Y is absent.

In general, in accordance with embodiments, R₁₀ and R₁₁ are independently selected from a group consisting of hydrogen, C₁-C₈ substituted or unsubstituted alkyl optionally substituted with at least one heteroatom, and C₆-C₁₄ substituted or unsubstituted aryl optionally substituted with at least one heteroatom. In some embodiments, R₁₀ and R₁₁ are independently selected from a group consisting of hydrogen, C₁-C₆ substituted or unsubstituted alkyl optionally substituted with at least one heteroatom; C₆-C₁₄ substituted or unsubstituted aryl optionally substituted with at least one hetero atom. In certain embodiments, R₁₀ and R₁₁ are independently selected from a group consisting of hydrogen, C₁-C₆ substituted or unsubstituted alkyl optionally substituted with at least one heteroatom; C₆-C₁₂ substituted or unsubstituted aryl optionally substituted with at least one hetero atom. In specific embodiments, and R₁₀ and R₁₁ is methyl.

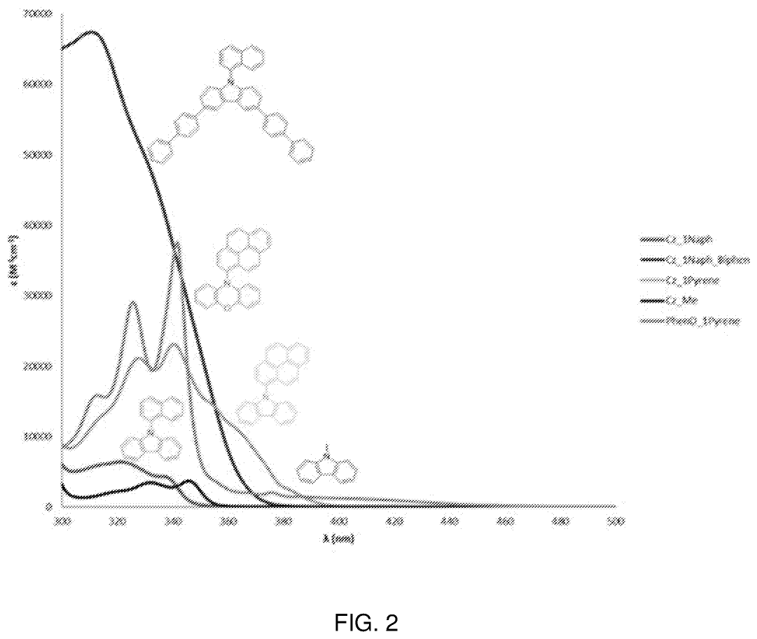

The compounds comprising Formula (IV) have a strong UV absorbance generally from about 300 nm to about 500 nm. In general, the compounds comprising Formula (IV) exhibit a strong molar extinction coefficient from about 25,000 M⁻¹cm⁻¹ to about 60,000 M⁻¹cm⁻¹. In various embodiments, the compounds comprising Formula (IV) have a strong UV absorbance. In general, the compounds comprising Formula (IV) exhibit a strong molar extinction coefficient from about 25,000 M⁻¹Cm⁻¹ to about 55,000 M⁻¹Cm⁻¹, from about 30,000 M⁻¹Cm⁻¹ to about 50,000 M⁻¹Cm⁻¹, or from about 40,000 M⁻¹Cm⁻¹ to about 48,000 M⁻¹Cm⁻¹. FIG. 2 shows the absorbance for three characteristic PCs.

Generally, the compounds comprising Formula (IV) exhibit E⁰*(PC^{●+}/³PC*) potentials ranging from about −2.30 V vs SCE (standard calomel electrode) to about −1.00 V vs SCE. In various embodiments, the compounds comprising Formula (IV) exhibit a E⁰*(PC^{●+}/³PC*) potentials ranging from about −2.30 V vs SCE (standard calomel electrode) to about −1.00 V vs SCE, from about −2.10 versus SCE to about −1.20 V vs. SCE, or from about −1.80 V vs. SCE to about −1.40 V versus SCE.

In general, the compounds comprising Formula (IV) exhibit Eₒₓ(PC^{●+}/PC) from about 0.71 vs. SCE to about 0.90 vs. SCE. In various embodiments, the compounds comprising Formula (IV) exhibit Eₒₓ(PC^{●+}/PC) from about 0.71 vs. SCE to about 0.90 vs. SCE, from about 0.75 vs. SCE to about 0.85 vs. SCE, or from about 0.78 vs. SCE to about 0.82 vs. SCE.

Generally, the compounds comprising Formula (IV) or salt thereof are known as organic photocatalysts (PCs) or organocatalyzed atom transfer radical polymerization (O-ATRP) catalysts.

### (II) Processes for the Preparation of Compounds Comprising Formula (IV)

In another aspect, the present disclosure provides processes to prepare compound comprising Formula (IV) or salt thereof:

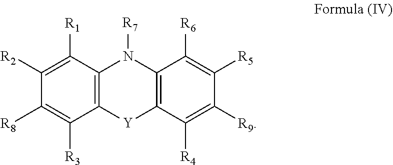

The processes comprising contacting the compound comprising Formula (I):

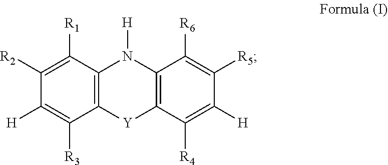

in the presence of an aromatic halide and a catalyst to form a compound comprising Formula (II):

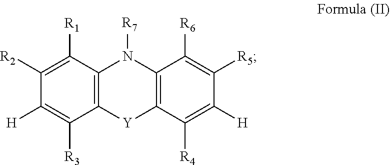

contacting the compound comprising Formula (II) with a halogenation reagent forming the compound comprising Formula (III):

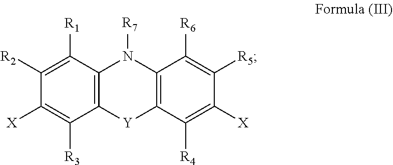

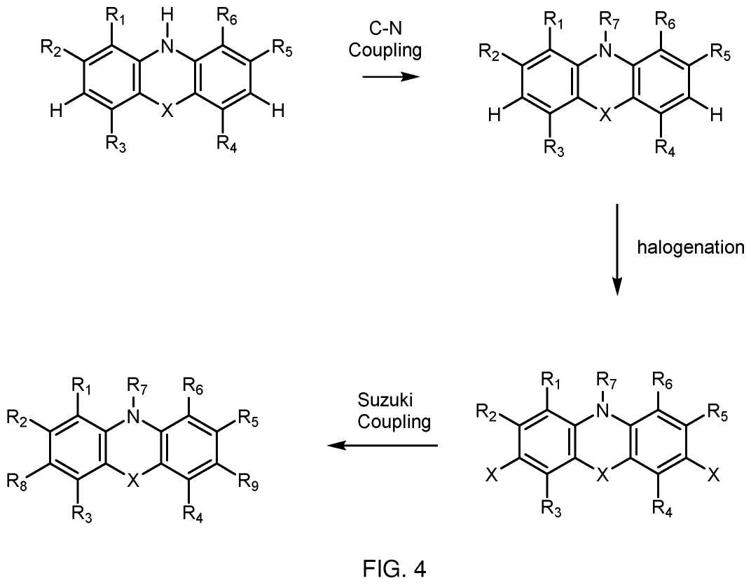

contacting the compound comprising Formula (III) with an aryl boronic acid in the presence of a catalyst to prepare the compound comprising Formula (IV), according to the Reaction Scheme depicted in FIG. 4.

Generally, in accordance with embodiments of the Reaction Scheme depicted in FIG. 4, R₁, R₂, R₃, R₄, R₅, and R₆ are independently selected from a group consisting of hydrogen, C₁-C₈ substituted or unsubstituted alkyl optionally substituted with at least one heteroatom; C₆-C₂₀ substituted or unsubstituted aryl optionally substituted with at least one hetero atom. In some embodiments, R₁, R₂, R₃, R₄, R₅, and R₆ are independently selected from a group consisting of hydrogen, C₁-C₆ substituted or unsubstituted alkyl optionally substituted with at least one heteroatom; C₆-C₁₄ substituted or unsubstituted aryl optionally substituted with at least one heteroatom. In certain embodiments, R₁, R₂, R₃, R₄, R₅, and R₆ are independently selected from a group consisting of hydrogen, C₁-C₄ substituted or unsubstituted alkyl optionally substituted with at least one heteroatom; C₆-C₁₄ substituted or unsubstituted aryl optionally substituted with at least one hetero atom. In specific embodiments, R₁, R₂, R₃, R₄, R₅, and R₆ are hydrogen.

In general, in accordance with embodiments of the Reaction Scheme depicted in FIG. 4, R₇ is selected group consisting of C₁-C₈ substituted or unsubstituted alkyl optionally substituted with at least one heteroatom; C₆-C₂₀ substituted or unsubstituted aryl optionally substituted with at least one hetero atom. In some embodiments, R₇ is selected from a group consisting of C₁-C₆ substituted or unsubstituted alkyl optionally substituted with at least one heteroatom; C₆-C₁₄ substituted or unsubstituted aryl optionally substituted with at least one heteroatom. In certain embodiments, R₇ is selected from a group consisting of C₁-C₄ substituted or unsubstituted alkyl optionally substituted with at least one heteroatom C₆-C₁₄ substituted or unsubstituted aryl optionally substituted with at least one heteroatom. In specific embodiments, R₇ is selected from a group consisting of methyl, ethyl, phenyl, 1-naphthyl, 2-naphthyl, phenyl, 4-methoxyphenyl, or 4-cyanophenyl.

Generally, in accordance with embodiments of the Reaction Scheme depicted in FIG. 4, R₈ and R₉ are independently selected from a group consisting of C₆-C₂₀ substituted or unsubstituted aryl optionally substituted with at least one hetero atom. In certain embodiments, R₈ and R₉ are independently selected from a group consisting of C₆-C₂₀ substituted or unsubstituted aryl optionally substituted with at least one hetero atom. In some embodiments, R₈ and R₉ are independently selected from a group consisting of C₆-C₂₀ substituted or unsubstituted aryl optionally substituted with at least one hetero atom. In specific embodiments, R₈ and R₉ are independently selected from a group consisting of 4, 4′-biphenyl, 4-methoxyphenyl, or 4-cyanophenyl.

In general, in accordance with embodiments of the Reaction Scheme depicted in FIG. 4, Y is O, CR₁₀R₁₁, or absent. In specific embodiments, Y is O, CR₁₀R₁₁, or absent.

Generally, in accordance with embodiments of the Reaction Scheme depicted in FIG. 4, R₁₀ and R₁₁ are independently selected from a group consisting of hydrogen, C₁-C₈ substituted or unsubstituted alkyl optionally substituted with at least one heteroatom, and C₆-C₁₄ substituted or unsubstituted aryl optionally substituted with at least one heteroatom. In some embodiments, R₁₀ and R₁₁ are independently selected from a group consisting of hydrogen, C₁-C₆ substituted or unsubstituted alkyl optionally substituted with at least one heteroatom; and C₆-C₁₄ substituted or unsubstituted aryl optionally substituted with at least one hetero atom. In certain embodiments, R₁₀ and R₁₁ are independently selected from a group consisting of hydrogen, C₁-C₆ substituted or unsubstituted alkyl optionally substituted with at least one heteroatom; C₆-C₁₂ substituted or unsubstituted aryl optionally substituted with at least one hetero atom. In specific embodiments, and R₁₀ and R₁₁ is methyl.

In general, in accordance with embodiments of the Reaction Scheme depicted in FIG. 4, X is independently Cl, Br, or I. In some embodiments, X is independently Cl, Br, or I. In specific embodiments, X is Br.

### (a) C—N Cross Coupling Reaction of Step (a).

As discussed above, Step (a) of the three step process involves contacting the compound comprising Formula (I) with an aryl halide in the presence of a catalyst. Contacting between the compound comprising Formula (I) with the aryl halide in the presence of a catalyst entails the cross coupling of the nitrogen atom on the compound comprising Formula (I) with carbon attached to the halide on the aromatic halide. The catalyst may further comprise a ligand, or be considered a pre-catalyst of the active catalyst. This reaction is termed the “Buchwald-Hartwig Amination.”

The compound comprising Formula (I) is detailed above. In some embodiments, R₁, R₂, R₃, R₄, R₅, and R₆ are independently selected from a group consisting of hydrogen, C₁-C₆ substituted or unsubstituted alkyl optionally substituted with at least one heteroatom; C₆-C₁₄ substituted or unsubstituted aryl optionally substituted with at least one hetero atom. In certain embodiments, R₁, R₂, R₃, R₄, R₅, and R₆ are independently selected from a group consisting of hydrogen, C₁-C₄ substituted or unsubstituted alkyl optionally substituted with at least one heteroatom; C₆-C₁₄ substituted or unsubstituted aryl optionally substituted with at least one hetero atom. In some embodiment, Y is O, CR₁₀R₁₁, or absent. In some embodiments, R₁₀ and Ru are independently selected from a group consisting of hydrogen, C₁-C₆ substituted or unsubstituted alkyl optionally substituted with at least one heteroatom; and C₆-C₁₄ substituted or unsubstituted aryl optionally substituted with at least one heteroatom. In certain embodiments, Y is O, CR₁₀R₁₁, or absent. In certain embodiments, R₁₀ and Ru are independently selected from a group consisting of hydrogen, C₁-C₆ substituted or unsubstituted alkyl optionally substituted with at least one heteroatom; and C₆-C₁₂ substituted or unsubstituted aryl optionally substituted with at least one heteroatom. Non-limiting examples of the compound comprising Formula (I) may be 3,7-bis([1,1′-biphenyl]-4-yl)-10-(1-naphthalenyl)-10H-phenoxazine, 3,6-bis([1,1′-biphenyl]-4-yl)-9-(1-naphthalenyl)-9H-carbazole, 9-(1-naphthalenyl)-3,6-diphenyl-9H-carbazole, 9-(1-naphthalenyl)-2,3,6,7-tetraphenyl-9H-carbazole, 9,9-dimethyl-9,10-dihydroacridine, 9-ethyl-9,10-dihydro-9-methylacridine, 9,9-diethyl-9,10-dihydroacridine, 9-(1,1-dimethylethyl)-9,10-dihydro-9-methylacridine, 9,10-dihydro-9,9-dipropylacridine, or 9,9-dibutyl-9,10-dihydroacridine. In one preferred embodiment, the compound comprising Formula (I) is 9,9-dimethyl-9,10-dihydroacridine.

Numerous aromatic halides are useful in the above-described process. The aromatic halide in the presence of the catalyst cross couples the aromatic group on the nitrogen of the compound comprising Formula (I). The aromatic halide comprises a chloride, a bromide, an iodide, or a triflate. The aromatic halides may be freshly prepared or purchased commercially. Non-limiting examples of suitable aromatic halides may be 1-bromonaphthalene, 2-bromonaphthalene, 4-bromobenzonitrile, 4-iodobenzonitrile, 4-bromoanisole, bromobenzene, or iodobenzene.

Generally, the mole ratio of the compound comprising Formula (I) with the aromatic halide in step (a) ranges from about 1.0:1.0 to about 1.0:2.0. In various embodiments, mole ratio of the compound comprising Formula (I) with the aromatic halide in step (a) ranges from about 1.0:1.0 to about 1.0:2.0, from about 1.0:1.05 to about 1.0:1.8, or from 1.0:1.25 to about 1.0:1.75.

The process detailed above, utilizes a catalyst or a precatalyst. In various embodiments, the catalyst useful in Step (a) may further comprises a ligand. In one embodiment, the catalyst or precatalyst useful in the above process is a palladium catalyst. Non-limiting examples of suitable palladium catalysts may be Pd₂(dba)₃, PdCl₂(P(o-tolyl₃)₂, Pd(OAc)₂, Pd(PPh₃)₄, or (diphenylphosphinoferrocene)PdCl₂. Non-limiting examples of suitable ligands may be PPh₃, P(o-tolyl)₃, P(t-Bu)₃, Brettphos, BINAP, Xphos, or Ruphos.

In general, the mole ratio of the compound comprising Formula (I) to the compound comprising Formula (I) may range from 1.0:0.001 to about 1.0:0.05. In various embodiments, the mole ratio of the catalyst to the compound comprising formula (I) may range from 1.0:0.001 to about 1.0:0.05, from about 1.0:0.005 to about 1.0:0.025, or from about 1.0:0.01 to about 1.0:0.02.

Generally, if the catalyst does not contain a ligand, the mole ratio of the catalyst to the ligand may range from about 1.0:0.5 to about 1.0:5.0. In various embodiments, the mole ratio of the catalyst to the ligand may range from about 1.0:0.5 to about 1.0:5.0, from about 1.0:1.0 to about 1.0:4.0, or from about 1.0:1.5 to about 1.0:3.0.

The process detailed above further comprises a base. Non-limiting examples of suitable bases may be NaOt-Bu, LHMDS, Cs₂CO₃, K₃PO₄, or K₂CO₃. In one embodiment, the base useful in the process is NaOt-Bu.

In general, the mole ratio of the compound comprising Formula (I) to the base may range from about 1.0:0.5 to about 1.0:5.0. In various embodiments, the mole ratio of the compound comprising Formula (I) to the base may range from about 1.0:0.5 to about 1.0:5.0, from about 1.0:1.0 to about 1.0:4.0, or from about 1.0:2.5 to about 1.0:3.5.

Step (a), as detailed herein, comprise a solvent. As recognized by those of skill in the art, the solvent can and will vary depending on the starting substrates, the catalyst, the ligand, the base, and the aromatic halide used in the process. The solvent may be a polar protic solvent, a polar aprotic solvent, a non-polar solvent, or combinations thereof. Suitable examples of polar protic solvents include, but are not limited to, water; alcohols such as methanol, ethanol, isopropanol, n-propanol, isobutanol, n-butanol, s-butanol, t-butanol, and the like; diols such as propylene glycol; organic acids such as formic acid, acetic acid, and so forth; amines such as trimethylamine, or triethylamine, and the like; amides such as formamide, acetamide, and so forth; and combinations of any of the above. Non-limiting examples of suitable polar aprotic solvents include acetonitrile, dichloromethane (DCM), diethoxymethane, N,N-dimethylacetamide (DMAC), N,N-dimethylformamide (DMF), dimethyl sulfoxide (DMSO), N,N-dimethylpropionamide, 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, bis(2-methoxyethyl)ether, 1,4-dioxane, N-methyl-2-pyrrolidinone (NMP), ethyl formate, formamide, hexamethylphosphoramide, N-methylacetamide, N-methylformamide, methylene chloride, nitrobenzene, nitromethane, propionitrile, sulfolane, tetramethylurea, tetrahydrofuran (THF), 2-methyltetrahydrofuran, trichloromethane, and combinations thereof. Suitable examples of non-polar solvents include, but are not limited to, alkane and substituted alkane solvents (including cycloalkanes), aromatic hydrocarbons, esters, ethers, combinations thereof, and the like. Specific non-polar solvents that may be employed include, for example, benzene, butyl acetate, t-butyl methylether, chlorobenzene, chloroform, chloromethane, cyclohexane, dichloromethane, dichloroethane, diethyl ether, ethyl acetate, diethylene glycol, fluorobenzene, heptane, hexane, isopropyl acetate, methyltetrahydrofuran, pentyl acetate, n-propyl acetate, tetrahydrofuran, toluene, and combinations thereof. In one exemplary embodiment, the solvent may be a combination of aprotic solvents. In one preferred embodiment, the solvent used in the process may be toluene.

In general, the volume to weight ratio of the solvent to the compound comprising Formula (I) will range from about 0.5:1 to about 500:1. In various embodiments, the volume to weight ratio of the solvent to the compound comprising Formula (I) may range from about 0.5:1 to about 500:1, from about 2:1 to about 250:1, from about 5:1 to about 200:1, or from about 10:1 to about 100:1. In an exemplary embodiment, the volume to weight ratio of the solvent to the compound comprising Formula (I) may range from about 20:1 to about 75:1.

In general, the reaction of step (a) will be conducted at a temperature that ranges from about 50° C. to about 150° C. depending on the solvent utilized. In various embodiments, the temperature of the reaction may range from about 50° C. to about 150° C., from about 70° C. to about 130° C., or from about 90° C. to about 120° C. In one embodiment, the reaction may be conducted at temperature that ranges from about 100° C. to about 120° C. The reaction typically is performed under ambient pressure. The reaction may also be conducted under an inert atmosphere, for example under nitrogen, argon or helium.

Generally, the reaction is allowed to proceed for a sufficient period of time until the reaction is complete, as determined by any method known to one skilled in the art, such as HPLC, TLC, or proton nuclear magnetic resonance (e.g., ¹H NMR). The duration of the reaction may range from about 5 minutes to about 24 hours. In some embodiments, the duration of the reaction may range from about 5 minutes to about 30 minutes, from about 30 minutes to about 2 hours, from about 2 hours to about 4 hours, from about 4 hours to about 10 hours, from about 10 hours to about 15 hours, or from about 15 hours to about 24 hours. In an exemplary embodiment, the reaction may be allowed to proceed for about 10 hours to about 16 hours. In this context, a “completed reaction” generally means that the reaction mixture contains a significantly diminished amount of the compound of Formula (I). Typically, the amount of the compound of Formula (I) remaining in the reaction mixture at the end of the reaction may be less than about 10%, less than about 5%, or less than about 2%.

The compound comprising Formula (II) may have a yield of at least about 60%. In various embodiments, the compound comprising Formula (II) may have a yield of at least about 60%, at least about 65%, at least about 70%, at least about 75%, at least about 80%, at least about 85%, at least about 90%, at least about 95%, or at least about 99%.

### (b) Halogenation of the Compound Comprising Formula (H)

As discussed above, Step (b) of the three step process involves contacting the compound comprising Formula (II) with halogenating agent. Contacting between the compound comprising Formula (II) with the halogenating agent entails substituting two halogen atoms for 2 hydrogens on the dihydroacridine ring forming the compound comprising Formula (III).

The compound comprising Formula (II) and Formula (III) are detailed above and herein. In some embodiments, X is independently Cl, Br, or I. In specific embodiments, X is Br.

A number of useful halogenation agents may be used in Step (b) of the process. Non-limiting examples of these halogenating agents may be N-chlorosuccinimide, N-bromosuccinimide, N-iodosuccinimide, bromine, or iodine. In one preferred embodiment, the halogenation agent used in Step (b) is N-bromosuccinimide.

As appreciated by the skilled artisan, the reaction halogenates two positions on the dihydroacridine ring. Generally, the mole ratio of the compound comprising Formula (II) and the halogenation reagent may range from about 1.0:2.0 to about 1.0:5.0. In various embodiments, the mole ratio of the compound comprising Formula (II) and the halogenation reagent may range from about 1.0:2.0 to about 1.0:5.0, from about 1.0:2.5 to about 1.0:4.0, or from about 1.0:3.0 to about 1:3.5.

Step (b) of the process further comprises a solvent. Solvents are listed above in Step (a). In one preferred embodiment, the solvent useful in the halogenation reaction is tetrahydrofuran (THF).

In general, the volume to weight ratio of the solvent to the compound comprising Formula (II) will range from about 1.0:1.0 to about 100:1.0. In various embodiments, the volume to weight ratio of the solvent to the compound comprising Formula (II) may range from about 1.0:1.0 to about 100:1.0, from about 5.0 to about 75.0:1.0, from about 10.0:1.0 to about 60.0:1.0, or from about 20.0:1.0 to about 50.0:1.0.

In general, the reaction of step (b) will be conducted at a temperature that ranges from about −10° C. to about 80° C. In various embodiments, the temperature of the reaction may range from about −10° C. to about 80° C., from about 0° C. to about 60° C., from about 10° C. to about 50° C., or from about 20° C. to about 30° C. In one embodiment, the reaction may be conducted at temperature that ranges from about 10° C. to about 40° C., or from about 20° C. to about 30° C. In another embodiment, the temperature of the reaction may be about room temperature (˜23° C.). The reaction typically is performed under ambient pressure. The reaction may also be conducted under an inert atmosphere, for example under nitrogen, argon or helium.

Generally, the reaction is allowed to proceed for a sufficient period of time until the reaction is complete, as determined by any method known to one skilled in the art, such as HPLC or proton nuclear magnetic resonance (e.g., ¹H NMR). The duration of the reaction may range from about 5 minutes to about 24 hours. In some embodiments, the duration of the reaction may range from about 5 minutes to about 30 minutes, from about 30 minutes to about 2 hours, from about 2 hours to about 4 hours, from about 4 hours to about 10 hours, from about 10 hours to about 15 hours, or from about 15 hours to about 24 hours. In an exemplary embodiment, the reaction may be allowed to proceed for about 1.0 hour to about 2 hours. In this context, a “completed reaction” generally means that the reaction mixture contains a significantly diminished amount of the compound of Formula (II). Typically, the amount of the compound of Formula (II) remaining in the reaction mixture at the end of the reaction may be less than about 10%, less than about 5%, or less than about 2%.

The compound comprising Formula (III) may have a yield of at least about 60%. In various embodiments, the compound comprising Formula (II) may have a yield of at least about 60%, at least about 65%, at least about 70%, at least about 75%, at least about 80%, at least about 85%, at least about 90%, at least about 95%, or at least about 99%.

### (c) Cross Coupling of the Compound Comprising Formula (III)

As discussed above, Step (c) of the three step process involves contacting the compound comprising Formula (III) with a functionalized organic aromatic boron compound in the presence of catalyst and a base. Contacting between the compound comprising Formula (III) with organic aromatic boron compound, a catalyst, and a base entails substituting two halogen atoms for 2 aromatic groups on the dihydroacridine ring forming the compound comprising Formula (IV). As appreciated by the skilled artisan, this process step is termed a “Suzuki Cross-Coupling Reaction” or a “Suzuki-Miyaura Cross-Coupling Reaction.”

The compound comprising Formula (III) is described in more detail herein and above.

A wide variety of organic aromatic boron compounds may be used in Step (c). These organic aromatic boron compounds may be boronic acids, boronic acid ester, a protected boronic acid, or trifluoroborates. Non-limiting examples of useful organic aromatic boron compounds may be 4-biphenylboronic acid, potassium (4-cyanophenyl)-trifluoroborate, 4-methoxyphenylboronic acid, 4-(4,4,5,5-tetramethyl-1,3,2-dioxaborolan-2-yl)anisole, 4-cyanobenzene boronic acid, or 4-methoxybenzeneboronic acid.

In general, the mole ratio of the compound comprising Formula (III) to the organic aromatic boron compound may range from about 1.0:2.0 to about 1.0:10.0. In various embodiments, the mole ratio of the compound comprising Formula (III) to the organic aromatic boron compound may range from about 1.0:2.0 to about 1.0:10.0, from about 1.0:3.0 to about 1.0:8.0, from about 1.0:4.0 to about 1.0:6.0. In one embodiment, the mole ratio of the compound comprising Formula (III) to the organic aromatic boron compound may be about 1.0:4.0.

The process as detailed above, utilized a catalyst. The catalyst can comprise a palladium catalyst or a nickel catalyst. In various embodiments, the catalyst useful in Step (c) may further comprises a ligand. Non-limiting of suitable catalysts may be Pd(OAc)₂, Pd(OCOCF₃)₂, Pd(PPh₃)₂Cl₂, Pd(PPh₃)₄, or Ni(PPh₃)₂Cl₂. Non-limiting examples of suitable ligands may be PPh₃, P(O-tolyl)₃, BINAP, BINAM.

In general, the mole ratio of the compound comprising Formula (III) to the catalyst may range from 1.0:0.01 to about 1.0:0.20. In various embodiments, the mole ratio of the catalyst to the compound comprising Formula (III) may range from 1.0:0.01 to about 1.0:0.20, from about 1.0:0.05 to about 1.0:0.17, or from about 1.0:0.07 to about 1.0:0.15. In one embodiment, the mole ratio of the catalyst to the compound comprising Formula (III) is about 1.0:0.15.

Generally, if the catalyst does not contain a ligand, the mole ratio of the catalyst to the ligand may range from about 1.0:0.5 to about 1.0:5.0. In various embodiments, the mole ratio of the catalyst to the ligand may range from about 1.0:0.5 to about 1.0:5.0, from about 1.0:1.0 to about 1.0:4.0, or from about 1.0:1.5 to about 1.0:3.0.

The process detailed above further comprises a base. The base may be a solid base or dissolved in water at various concentrations. Non-limiting examples of suitable bases may be NaOt-Bu, LHMDS, Cs₂CO₃, K₃PO₄, or K₂CO₃. In one preferred embodiment, the base useful in Step (c) is K₂CO₃.

In general, the mole ratio of the compound comprising Formula (III) to the base may range from 1.0:5.0 to about 1.0:50.0. In various embodiments, the mole ratio of the compound comprising Formula (III) to the base may range from 1.0:5.0 to about 1.0:50.0, from about 1.0:10.0 to about 1.0:40.0, or from about 1.0:20.0 to 1.0:30.0.

Step (c) of the process further comprises a solvent. Solvents are listed above in Step (a). In one preferred embodiment, the solvent useful in the halogenation reaction is tetrahydrofuran (THF) in a mixture with water.

In general, the volume to weight ratio of the solvent to the compound comprising Formula (II) will range from about 1.0:1.0 to about 100:1.0. In various embodiments, the volume to weight ratio of the solvent to the compound comprising Formula (II) may range from about 1.0:1.0 to about 100:1.0, from about 5.0 to about 75.0:1.0, from about 10.0:1.0 to about 60.0:1.0, or from about 20.0:1.0 to about 50.0:1.0.

In general, the reaction of step (c) will be conducted at a temperature that ranges from about 25° C. to about 100° C. In various embodiments, the temperature of the reaction may range from about 25° C. to about 100° C., from about 30° C. to about 90° C., from about 40° C. to about 80° C., or from about 60° C. to about 70° C. In one embodiment, the reaction may be conducted at temperature where the Step (c) refluxes (˜66° C.). The reaction typically is performed under ambient pressure. The reaction may also be conducted under an inert atmosphere, for example under nitrogen, argon or helium.

Generally, the reaction is allowed to proceed for a sufficient period of time until the reaction is complete, as determined by any method known to one skilled in the art, such as HPLC or proton nuclear magnetic resonance (e.g., ¹H NMR). The duration of the reaction may range from about 5 minutes to about 72 hours. In some embodiments, the duration of the reaction may range from about 5 minutes to about 72 hours, from about 1.0 hour to about 60 hours, from about 12 hours to about 55 hours, or from about 30 hours to about 50 hours. In an exemplary embodiment, the reaction may be allowed to proceed for about 40 hours to about 60 hours. In this context, a “completed reaction” generally means that the reaction mixture contains a significantly diminished amount of the compound of Formula (III). Typically, the amount of the compound of Formula (III) remaining in the reaction mixture at the end of the reaction may be less than about 10%, less than about 5%, or less than about 2%.

The compound comprising Formula (III) may have a yield of at least about 60%. In various embodiments, the compound comprising Formula (III) may have a yield of at least about 60%, at least about 65%, at least about 70%, at least about 75%, at least about 80%, at least about 85%, at least about 90%, at least about 95%, or at least about 99%.

(III) A Method for Preparing an Acrylate Polymer with a High Propagation Constant

In another aspect, the present disclosure provides methods for preparing non-statistical, linear polymers. the method comprising: (a) generating a reaction mixture comprising contacting monomer A, the compound comprising Formula (IV), an initiator, a salt additive, and a solvent; (b) irradiating the reaction mixture with UV light to generate the linear polymer In-Aₙ-X; (c) isolating at least a portion of linear polymer In-Aₙ-X; (d) generating a second reaction mixture comprising the linear polymer In-Aₙ-X from step (c), monomer B, a salt additive, comprising Formula (IV), and a solvent; (e) irradiating the second reaction mixture with UV light to form a linear polymer In-Aₙ-Bₘ—X; and (f) isolating at least a portion of linear polymer In-Aₙ-Bₘ—X wherein n and m are integers ranging from 1 to 10,000 and X is Cl, Br, or I. The method further comprising preparing a third reaction mixture comprising contacting the linear polymer In-Aₙ-Bₘ—X, monomer C, a salt additive, comprising Formula (IV), and a solvent; irradiating the third reaction mixture with UV light; and isolating at least a portion of the linear polymer In-Aₙ-Bₘ—Cₒ—X wherein o is an integer from 1 to 10,000 and X is Cl, Br, or I. In the above aspect, the linear polymers are non-statistical, high degree of control over molecular weight (I* close to 100%), and a moderate to low dispersity (D).

In one embodiment, the method may be conducted in a batch reactor. In another embodiment, the method may be conducted in a continuous flow reactor.

The compound comprising Formula (IV) is described above.

Generally, the monomers A, B, and C may be the same or different. In one embodiment, the non-statistical, linear polymer may be a homopolymer. As appreciated by the skilled artisan, the homopolymer consists of the same monomer throughout the linear polymer. In another embodiment, the linear polymer may be a copolymer. As defined herein, a copolymer comprises at least two different monomers in the linear polymer.

Various monomers are useful in these methods. Generally, monomers A, B, and C are independently selected from a group consisting of an acrylate ester, an acrylic acid, acrylonitrile, methacrylate ester, methacrylic acid, or methacrylonitrile. In various embodiments, the ester portion of the acrylate or methacrylate ester is less than 10 carbons. Non-limiting examples of these acrylate or methacrylate esters may be methyl acrylate, ethyl acrylate, methyl methacrylate, ethyl acrylate, propyl acrylate, i-propyl acrylate, butyl acrylate, t-butyl acrylate, 2-hydroxyethyl acrylate, benzyl acrylate, or t-butyl acrylate.

In general, the equivalent ratio of the monomers A, B, or C to the compound of claim 1 may range from about 1.0:1.0 to about 10,000:1.0. In various embodiments, the equivalent ratio of the monomers A, B, or C to the compound of claim 1 may range from about 1.0:1.0 to about 10,000.0:1.0, from about 10.0:1.0 to about 5,000.0:1.0, from about 100.0:1.0 to about 1,000.0:1.0.

The method comprises an initiator (In). The initiator consisting of a bromide, once contacted with the compound of claim 1 initiates the polymerization of the monomer. Non-limiting examples of initiators may be methyl α-bromoisobutyrate (MBiB), diethyl 2-bromo-2-methylmalonate (DBMM), 2-bromopropionnitrile (2PN), tert-butyl α-bromoisobutyrate, methyl 2-bromopropionate (M2BP), and 2-bromopropionitrile (2BrCN). In one preferred embodiment, the initiator is diethyl 2-bromo-2-methylmalonate (DBMM).

Generally, the equivalent ratio of the initiator to the compound of claim 1 may range from about 1.0:1.0 to about 50.0:1.0. In various embodiments, the equivalent ratio of the initiator to the compound of claim 1 may range from 1.0:1.0 to about 50.0:1.0, from about 2.0:1.0 to about 40.0:1.0, from about 5.0:1.0 to about 25.0:1.0, or from about 8.0:1.0 to about 12.5:1.0. In one preferred embodiment, the equivalent ratio of the initiator to the compound of claim 1 is about 10.0:1.0.

The method further comprises a salt additive. The salt additive terminates the polymerization reaction. Non-limiting examples of suitable salt additives may be lithium bromide, sodium bromide, potassium bromide, tetrabutylammonium bromide, lithium chloride, sodium chloride, potassium chloride, lithium iodide, potassium iodide, lithium hexafluorophosphate. In one preferred embodiment, the salt additive is lithium bromide.

In general, the equivalent ratio of the salt additive to the compound of claim 1 may range from about 1.0:1.0 to about 50.0:1.0. In various embodiments, the equivalent ratio of the salt additive to the compound of claim 1 may range from 1.0:1.0 to about 50.0:1.0, from about 2.0:1.0 to about 40.0:1.0, from about 5.0:1.0 to about 25.0:1.0, or from about 8.0:1.0 to about 12.5:1.0. In one preferred embodiment, the equivalent ratio of the salt additive to the compound of claim 1 is about 10.0:1.0.

The method comprises a solvent. Suitable solvents are described above in Section (II). In preferred embodiments, the solvent utilized in the method comprises N, N-dimethylacetamide (DMAc), dimethylformamide (DMF), dimethylsulfoxide (DMSO), tetrahydrofuran (THF), or combinations thereof.

In general, the volume to volume ratio of the solvent to the monomer A, B, or C may range from 0.1:1.0 to about 10.0:1.0. In various embodiments, the volume to volume ratio of the solvent to the monomer A, B, or C may range from 0.1:1.0 to about 10.0:1.0, from about 0.5:1.0 to about 7.5:1.0, or from about 1.0:1.0 to about 2.0:1.0. In one embodiment, the volume to volume ratio of the solvent to the monomer A, B, or C may be about 1.5:1.0.

A wide variety of UV sources may be used in the method. Non-limiting sources of UV light may be natural sunlight, a gas discharge lamp, an incandescent lamp, or an LED (light-emitting diode).

Generally, the wavelength of UV light may range from about 350 nm to about 400 nm. In various embodiments, the wavelength of UV light may range from about 350 nm to about 380 nm, from about 355 nm to about 375 nm, or about 360 nm to about 370 nm. In one preferred embodiment, the wavelength useful in the method may be about 365 nm.

The temperature of the process can and will vary depending on the monomer, the compound of claim 1, the initiator, and the solvent. Generally, the temperature of the process may range from about 0° C. to about 50° C. In various embodiment, the temperature of the process may range from about 0° C. to about 50° C., from about 10° C. to about 40° C., or from about 18° C. to about 26° C. In one embodiment, the reaction may be conducted at temperature that ranges from about 10° C. to about 40° C., or from about 20° C. to about 30° C. In another embodiment, the temperature of the reaction may be about room temperature (˜22° C.). The reaction typically is performed under ambient pressure. The reaction may also be conducted under an inert atmosphere, for example under nitrogen, argon or helium.

Generally, the reaction is allowed to proceed for a sufficient period of time until the reaction is complete, as determined by any method known to one skilled in the art, such as SEC-MALS GPC (multi-angle light scattering coupled to size exclusion chromatography gel permeation chromatography) or proton nuclear magnetic resonance (e.g., ¹H NMR). The duration in forming the linear polymer depends on the concentration of the reactants in the method, the steric bulk of the monomer A, B, or C, the initiator, the salt additive, and the compound of claim 1. The duration of the reaction may range from about 1 minute to about 720 minutes. In some embodiments, the duration of the reaction may range from about from about 1 minute to about 720 minutes, from about 10 minutes to about 600 minutes, from about 60 minutes to about 480 minutes, or from about 120 minutes to about 360 minutes. In this context, a “completed reaction” generally means that the reaction mixture contains a large concentration of the linear polymer. Typically, the amount of the linear polymer in the reaction mixture at the end of the reaction may be greater than 40%, greater than 50%, or even greater than 75%. The amount of remaining monomer A, B, or C may be less than 20%, less than 15%, less than 10%, less than 5%, or less than 2%.

Generally, the monomers A, B, and C may be the same or different. In one embodiment, the non-statistical, linear polymer may be a homopolymer. As defined herein, a homopolymer consists of the same monomer throughout the linear polymer. In another embodiment, the linear polymer may be a copolymer. As defined herein, a copolymer comprises at least two different monomers in the linear polymer.

The relative length of the linear polymer is dependent on the compound of claim 1, the monomer A, B, or C used, the amount of monomer A, B, or C in the method, the solvent, the initiator, and the salt additive. Generally, the values of m, n, and o range from 1 to about 10,000.

The linear polymer may have a yield of at least about 20%. In various embodiments, the linear polymer may have a yield of at least about 20%, at least about 30%, at least about 50%, at least about 60%, at least about 70%, at least about 80%, at least about 90%, at least about 95%, or at least about 99%.

The linear polymer has a dispersity (D) less than or equal to 1.20.

The linear polymers have a high degree of control of molecular weight (I* close to 100%).

### (IV) Methods for Forming Aryl Carbon-Nitrogen Bonds Using Dual Nickel/Photoredox Catalyzed C—N Cross-Couplings

In yet other aspects of the disclosure, the compounds of Formula (IV), described herein above, may be used in dual catalytic methods for forming an aryl carbon-nitrogen bond. In certain embodiments, the dual catalytic methods may comprise contacting an aryl halide with an amine in the presence of a dual catalytic solution comprising a Ni(II) salt catalyst, a compound of Formula (IV) of the disclosure, and an optional base, thereby forming a reaction mixture; and exposing the reaction mixture to light under reaction conditions sufficient to form the aryl carbon-nitrogen bond. Without intending to be limited by theory, the compounds of Formula (IV) of the disclosure function as photoredox catalysts in the C—N cross-coupling reactions.

Suitable reaction components and parameters for forming aryl carbon-nitrogen bonds are detailed below. In accordance with certain aspects, the present disclosure provides a nickel-catalyzed C—N cross-coupling methodology that operates at room temperature in the presence of an inexpensive nickel source (e.g., a Ni bromide salt) and a compound of Formula (IV) described herein.

In certain embodiments, the light is visible light or UV light. In certain embodiments, the amine is present in a molar excess to the aryl halide. In certain embodiments, the Ni salt catalyst solution includes a Ni salt and a polar solvent, wherein the Ni salt is dissolved in the polar solvent. In other embodiments, the reaction mixture includes a polar solvent. In yet other embodiments, the reaction mixture may include a compound of Formula (IV) described herein as a PC.

In certain embodiments, the reactions conditions include holding the reaction mixture at suitable temperatures, e.g., between about room temperature and about 100° C., between room temperature and about 90° C., between about room temperature and about 80° C., etc., for between about 30 minutes and about 20 hours, for between about 1 hour and about 48 hours, 12 to 24 hours, etc., such that at least about 50% yield, at least about 55% yield, at least about 60% yield, etc. is obtained.

In certain embodiments, the amine may be present in a molar excess to the aryl halide. In certain embodiments, the Ni salt may be a nickel bromide salt such as NiBr₂.3H₂O salt. In certain embodiments, the optional base may be an amine containing base such as quinuclidine. In certain embodiments, the Ni salt catalyst solution includes a polar solvent, where the Ni salt is in the polar solvent. In other embodiments, the reaction mixture includes a polar solvent. In certain embodiments, the polar solvent may be N,N-dimethylacetamide. In certain embodiments, the light may be visible light or UV light, e.g., 365 nm, 405 nm, 457 nm, 523 nm, etc.

In certain embodiments, the aryl halide may be selected from the group consisting of an aryl bromide, an aryl chloride, and an aryl iodide. By way of example, the aryl halide may be selected from the group consisting of bromobenzene; 4-bromobenzotrifluoride; 3-bromobenzotrifluoride; 1-bromo-3,5-diflurobenzene; 4-bromobenzofluoride; 1-bromo-3-(trifluoromethyl)benzene; 1-bromo-3-chlorobenzene; 4-bromobenzamide; 1-bromo-4-methylbenzene; 1-bromo-4-methoxybenzene; 1-bromo-3-methoxybenzene; 1-bromo-3,5-dimethoxybenzene; 4-bromobenzonitrile; methyl 4-bromobenzoate; 1-(4-bromophenyl)ethan-1-one; 3-bromopyridine; 5-bromopyrimidine; chlorobenzene; 4-chlorobenzotrifluoride; 3-chlorobenzotrifluoride; 1-chloro-3,5-diflurobenzene; 4-chlorobenzofluoride; 1-chloro-3-(trifluoromethyl)benzene; 1-chloro-3-chlorobenzene; 4-chlorobenzamide; 1-chloro-4-methylbenzene; 1-chloro-4-methoxybenzene; 1-chloro-3-methoxybenzene; 1-chloro-3,5-dimethoxybenzene; 4-chlorobenzonitrile; methyl 4-chlorobenzoate; 1-(4-chlorophenyl)ethan-1-one; 3-chloropyridine; 5-chloropyrimidine; iodobenzene; 4-iodobenoztrifluoride; 3-iodobenzotrifluoride; 1-iodo-3,5-diflurobenzene; 4-iodobenzofluoride; 1-iodo-3-(trifluoromethyl)benzene; 1-iodo-3-chlorobenzene; 4-iodobenzamide; 1-iodo-4-methylbenzene; 1-iodo-4-methoxybenzene; 1-iodo-3-methoxybenzene; 1-iodo-3,5-dimethoxybenzene; 4-iodobenzonitrile; methyl 4-iodobenzoate; 1-(4-bromophenyl)ethan-1-one; 3-iodopyridine; 5-iodopyrimidine, and 2-iodotoluene.

In certain embodiments, the amine may be a primary amine or a secondary amine. By way of example, the amine may be selected from the group consisting of propylamine, cyclohexylamine, phenethylamine, pyridine-3-amine, furan-2-ylmethanamine, aniline, 4-fluoroaniline, pyrrolidine, piperidine, piperazine, tert-butyl piperazine-1-carboxylate, morpholine, 4-methyl-piperidine, piperdine-4-ol, piperidine-4-carbonitrile, methyl piperidine-4-carboxylate, cyclohexanamine, 3-aminopyridine, propan-1-amine, hexan-1-amine, 2-phenylethan-1-amine, and indoline. The amine may be present in a molar excess of the aryl halide present in the reaction mixture. For example, the amine may be present in about 1.0 to about 5.5 molar excess of the aryl halide present in the reaction mixture.

In certain embodiments, the Ni salt catalyst solution may comprise a Ni(II) salt. For example, the Ni(II) salt may be selected from the group consisting of Ammonium nickel(II) sulfate hexahydrate, Nickel(II) acetate tetrahydrate, Nickel(II) bromide anhydrous, Nickel(II) bromide, Nickel(II) bromide hydrate, Nickel carbonate, basic hydrate, Nickel(II) carbonate hydroxide tetrahydrate, Nickel(II) chloride anhydrous, Nickel(II) chloride, Nickel(II) fluoride, Nickel(II) hydroxide, Nickel(II) iodide anhydrous, Nickel(II) iodide, Nickel(II) nitrate hexahydrate, Nickel(II) perchlorate hexahydrate, Nickel(II) sulfamate tetrahydrate, Nickel(II) sulfate anhydrous, and Nickel(II) sulfate heptahydrate. In particular embodiments, the Ni(II) salt may be selected from the group consisting of NiBr₂.glyme, NiCl₂.6H2O, NiCl₂.glyme, and NiBr₂.3H₂O. By way of non-limiting example, the Ni(II) salt may be NiBr₂.3H₂O.

In certain embodiments, the dual catalyst solution further comprises a polar solvent, and the Ni(II) salt is dissolved in the polar solvent. By way of example, the polar solvent may be selected from the group consisting of N,N-dimethylacetamide, dimethyl sulfoxide, methanol, dimethylformamide, and acetonitrile. In particular embodiments, the polar solvent is N,N-dimethylacetamide.

In certain embodiments, the optional base may be selected from the group consisting of quinuclidine, morpholine, N,N-diisopropylethylamine, and triethylamine. In particular embodiments, the optional base is quinuclidine.

### Definitions

The term “acyl,” as used herein alone or as part of another group, denotes the moiety formed by removal of the hydroxy group from the group COOH of an organic carboxylic acid, e.g., RC(O)—, wherein R is R₁, R¹O—, R¹R²N—, or R¹S—, R¹ is hydrocarbyl, heterosubstituted hydrocarbyl, or heterocyclo, and R² is hydrogen, hydrocarbyl, or substituted hydrocarbyl.

The term “acyloxy,” as used herein alone or as part of another group, denotes an acyl group as described above bonded through an oxygen linkage (O), e.g., RC(O)O— wherein R is as defined in connection with the term “acyl.”

The term “alkyl” as used herein describes groups which are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “alkenyl” as used herein describes groups which are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The term “alkynyl” as used herein describes groups which are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “aromatic” as used herein alone or as part of another group denotes optionally substituted homo- or heterocyclic conjugated planar ring or ring system comprising delocalized electrons. These aromatic groups are preferably monocyclic (e.g., furan or benzene), bicyclic, or tricyclic groups containing from 5 to 14 atoms in the ring portion. The term “aromatic” encompasses “aryl” groups defined below.

The terms “aryl” or “Ar” as used herein alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

The terms “carbocyclo” or “carbocyclic” as used herein alone or as part of another group denote optionally substituted, aromatic or non-aromatic, homocyclic ring or ring system in which all of the atoms in the ring are carbon, with preferably 5 or 6 carbon atoms in each ring. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “halogen” or “halo” as used herein alone or as part of another group refer to chlorine, bromine, fluorine, and iodine.

The term “heteroatom” refers to atoms other than carbon and hydrogen.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms and/or 1 to 4 nitrogen atoms in the ring, and is bonded to the remainder of the molecule through a carbon. Exemplary groups include furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “heterocyclo” or “heterocyclic” as used herein alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include heteroaromatics as described above. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “oxygen protecting group” as used herein denotes a group capable of protecting an oxygen atom (and hence, forming a protected hydroxyl group), wherein the protecting group may be removed, subsequent to the reaction for which protection is employed, without disturbing the remainder of the molecule. Exemplary oxygen protecting groups include ethers (e.g., allyl, triphenylmethyl (trityl or Tr), p-methoxybenzyl (PMB), p-methoxyphenyl (PMP)), acetals (e.g., methoxymethyl (MOM), β-methoxyethoxymethyl (MEM), tetrahydropyranyl (TIP), ethoxy ethyl (EE), methylthiomethyl (MTM), 2-methoxy-2-propyl (MOP), 2-trimethylsilylethoxymethyl (SEM)), esters (e.g., benzoate (Bz), allyl carbonate, 2,2,2-trichloroethyl carbonate (Troc), 2-trimethylsilylethyl carbonate), silyl ethers (e.g., trimethylsilyl (TMS), triethylsilyl (TES), triisopropylsilyl (TIPS), triphenylsilyl (TPS), t-butyldimethylsilyl (TBDMS), t-butyldiphenylsilyl (TBDPS) and the like. A variety of oxygen protecting groups and the synthesis thereof may be found in “Greene's Protective Groups in Organic Synthesis,” 4^{th} Ed. by P. G. M. Wuts and T. W. Greene, John Wiley & Sons, Inc., 2007.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above-described methods without departing from the scope of the invention, it is intended that all matter contained in the above description and in the examples given below, shall be interpreted as illustrative and not in a limiting sense

### EXAMPLES

The following examples are included to demonstrate certain embodiments of the invention. It should be appreciated by those of skill in the art that the techniques disclosed in the examples represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth is to be interpreted as illustrative and not in a limiting sense.

### Instrumentation for Photocatalyst and Precursors Characterization:

Structural analysis was performed by a Varian 400 MHz NMR Spectrometer. UV-visible spectroscopy was carried out using a Cary 5000 UV-Vis-NIR spectrophotometer from Agilent. Fluorescence spectroscopy was performed using the FS5 Spectrofluorometer from Edinburg Instruments. Cyclic Voltammetry experiments were conducted using a Gamry Interface 1010B potentiostat.

### General Synthetic Scheme:

The following general synthetic scheme (FIG. 4) was used to prepare the organic photocatalysts (PCs) comprising Formula (IV).

### Example 1: Preparation of 9-(naphthalen-1-yl)-9H-carbazole

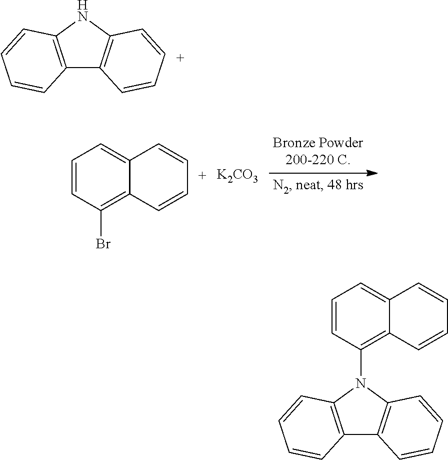

A storage tube was loaded with 1.500 g (8.97 mmol, 1 eq) carbazole, 6.275 mL 1-bromonaphthalene (44.85 mmol, 5.0 eq), 1.860 g K₂CO₃ (13.46 mmol, 1.5 eq), and 0.1495 g of bronze powder (1 g/62.5 mmol carbazole) and equipped with a stir bar. The reaction mixture was degassed and placed under a nitrogen atmosphere. The tube was then heated to 200-220° C. for 48 hours. The crude mixture obtained was dissolved in DCM, and filtered over excess MgSO4. The filtrate was concentrated by rotary evaporation and then purified by flash chromatography on silica gel. The product eluted with a gradient mobile phase of hexanes containing 0-10% ethyl acetate. Upon removing the solvent, 2.488 g (94.5% yield) of white solid was collected. ¹H NMR (300 MHz, Chloroform-d) δ 8.25-8.19 (m, 2H), 8.08-7.98 (m, 2H), 7.72-7.62 (m, 2H), 7.54 (ddd, J=8.2, 6.4, 1.6 Hz, 1H), 7.39-7.27 (m, 6H), 7.04-6.98 (m, 2H).

### Example 2: Preparation of 3,6-Dibromo-9-(naphthalen-1-yl)-9H-carbazole

Br

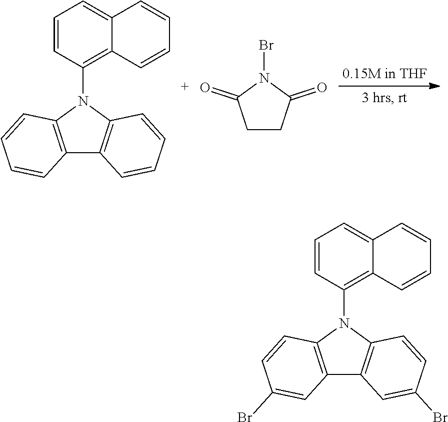 [SMILES: c1ccc2c(-n3c4ccccc4c4ccccc43)cccc2c1 ; O=C1CCC(=O)N1Br ; Brc1ccc2c(c1)c1cc(Br)ccc1n2-c1cccc2ccccc12]

round bottom as was loaded with 2.256 g (7.69 mmol, 1 eq) 9-napthalen-1-yl)-9H-carbazole and a stir bar, dissolved in THE to make a 0.15 M solution, and 3.011 g (16.92 mmol, 2.2 eq) N-bromosuccinimide was slowly added. The reaction was left to stir for 3 hours at room temperature. After the solvent was removed using a rotary evaporator, the remaining solid was dissolved in DCM and recrystallized by layering MeOH in a 3:1 DCM:MeOH mixture. 2.963 g of white crystals were recovered by filtration and dried (85.4% yield). ¹H NMR (300 MHz, Chloroform-d) δ 8.27 (dd, J=1.9, 0.5 Hz, 2H), 8.11-7.99 (m, 2H), 7.72-7.63 (m, 1H), 7.62-7.52 (m, 2H), 7.44 (dd, J=8.7, 1.9 Hz, 2H), 7.39-7.31 (m, 1H), 7.18-7.12 (m, 1H), 6.87 (dd, J=8.7, 0.5 Hz, 2H).

### Example 3: of 3,6-Di([1,1′-biphenyl]-4-yl)-9-(naphthalen-1-yl)-9H-carbazole

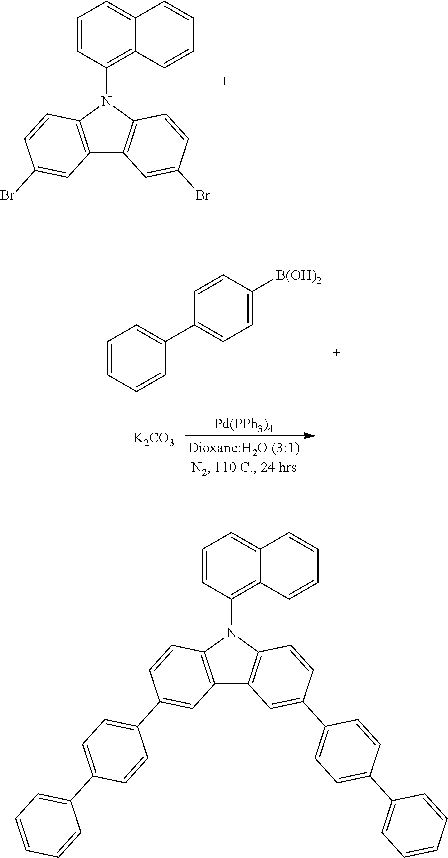

A storage tube was loaded with 0.500 g (1.11 mmol, 1.0 eq) of 3,6-dibromo-9-(naphthalen-1-yl)-9H-carbazole, 0.658 g (3.32 mmol, 3.0 eq) of [1,1′-biphenyl]-4-ylboronic acid, and 0.613 g (4.43 mmol, 4 eq) K₂CO₃. The tube was evacuated, filled with N₂, and brought into a N₂ filled glovebox where 0.0384 g (0.033 mmol, 0.03 eq) Pd(PPh₃)₄ was added. After removal from the glovebox, 5.55 mL dioxane and 1.85 mL H₂O were added under N₂ to form a 3:1 mixture, and the tube was heated to 110° C. for 24 hrs. The crude product was dissolved in DCM and extracted with deionized water using a separatory funnel. The organic layer was collected, washed with brine, dried with excess MgSO₄, and filtered. The mixture was then filtered through a silica plug, dried by rotary evaporation, and recrystallized using a 3:1 ratio of DCM:MeOH to obtain 0.490 g of white solid (73.8% yield). ¹H NMR (300 MHz, Chloroform-d) δ 8.59-8.51 (m, 2H), 8.15-8.02 (m, 2H), 7.90-7.80 (m, 4H), 7.77-7.64 (m, 12H), 7.63-7.54 (m, 1H), 7.53-7.44 (m, 4H), 7.43-7.33 (m, 4H), 7.11 (dd, J=8.5, 0.6 Hz, 2H). ¹³C NMR (75 MHz, Chloroform-d) δ 142.37, 141.06, 141.01, 139.57, 135.05, 134.02, 133.16, 131.02, 129.35, 128.96, 128.69, 127.80, 127.69, 127.38, 127.28, 127.19, 126.98, 126.85, 126.11, 125.79, 124.08, 123.61, 118.96, 110.84.

### Example 4: Preparation of 3,6-Dibromo-9H-carbazole

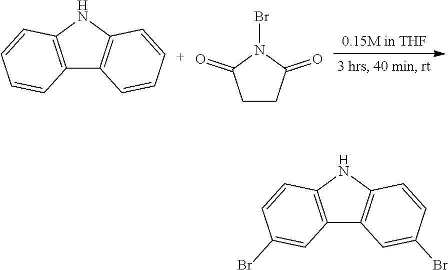

A round bottom flask was loaded with 1.000 g (5.98 mmol, 1 eq) carbazole and a stir bar, dissolved in THE to make a 0.15 M solution, and 2.342 g (13.16 mmol, 2.2 eq) N-bromosuccinimide was slowly added. The reaction was left to stir for 3 hours 40 minutes at room temperature. The solvent was removed by rotary evaporator and the crude product was dissolved with DCM and extracted with deionized H₂O. The organic layer was collected, this process was repeated once, and then the organic layer was washed with brine and dried with excess MgSO₄. After filtration and solvent removal, the crude solid residue was dissolved in 100 mL DCM and recrystallized by layering with 100 mL hexanes. 617 mg white powder was recovered, and a second recrystallization of the mother liquor produced 231 additional mg for a total of 0.849 g (43.8% yield) of 3,6-dibromo-9H-carbazole. ¹H NMR (300 MHz, Chloroform-d) δ 8.18-8.00 (m, 3H), 7.52 (dd, J=8.6, 1.9 Hz, 2H), 7.31 (dd, J=8.6, 0.6 Hz, 2H).

### Example 5: Preparation of 3,6-Di([1,1′-biphenyl]-4-yl)-9H-carbazole

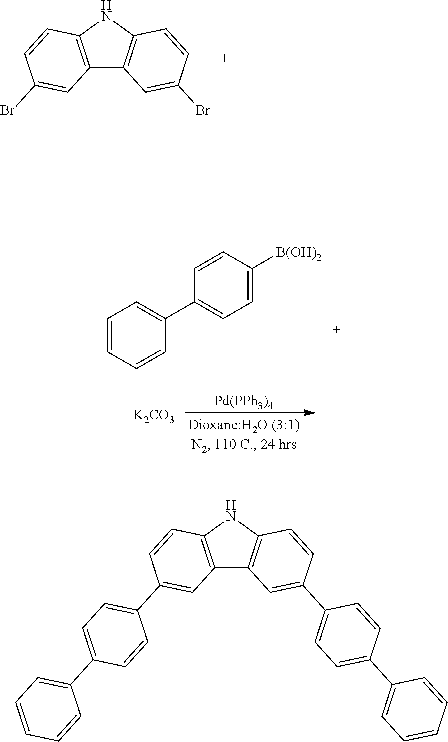

A storage tube was loaded with 0.500 g (1.54 mmol, 1.0 eq) of 3,6-dibromo-9H-carbazole, 0.914 g (4.62 mmol, 3.0 eq) of [1,1′-biphenyl]-4-ylboronic acid, and 0.851 g (6.16 mmol, 4 eq) K₂CO₃. The tube was evacuated, filled with N₂, and brought into a N₂ filled glovebox where 0.0534 g (0.046 mmol, 0.03 eq) Pd(PPh₃)₄ was added. After removal from the glovebox, 15.4 mL dioxane and 5.13 mL H₂O were added under N₂ to form a 3:1 mixture, and the tube was heated to 110° C. for 24 hrs. The crude product was dissolved in DCM and extracted with deionized water using a separatory funnel. The organic layer was collected, washed with brine, dried with excess MgSO₄, and filtered. The mixture was then filtered through a silica plug, dried by rotary evaporation, and dissolved toluene with heating at 50° C. The mixture was then separated using flash chromatography on silica gel, eluting with a gradient of 30%-100% EtAc in hexanes. The product eluted last but contained significant amounts of H₂O. After removing the solvent, the purified product was dissolved in DCM and dried with MgSO₄. After recrystallization from hot DCM, 0.301 g of white solid was obtained (41.5% yield). ¹H NMR (300 MHz, DMSO-d₆) δ 11.44 (s, 1H), 8.69 (d, J=1.7 Hz, 2H), 8.01-7.87 (m, 4H), 7.87-7.70 (m, 10H), 7.61 (d, J=8.6 Hz, 2H), 7.56-7.46 (m, 4H), 7.45-7.34 (m, 2H).

### Example 6: Preparation of 3,6-Di([1,1′-biphenyl]-4-yl)-9-(pyren-1-yl)-9H-carbazole

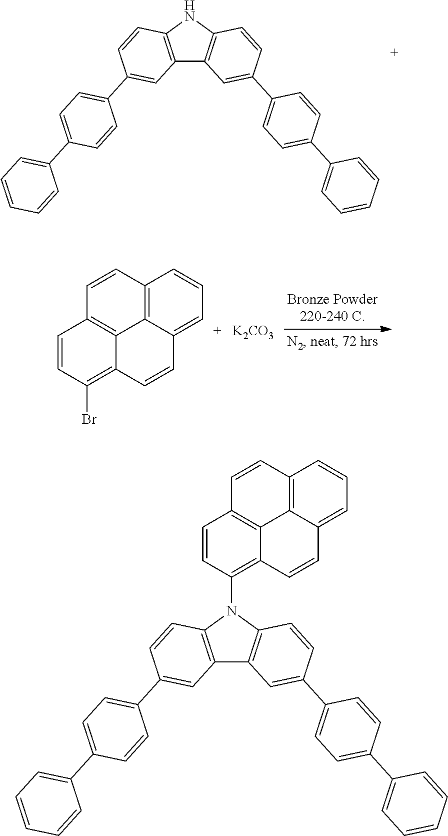

A storage tube was loaded with 0.500 g (1.06 mmol, 1 eq) of 3,6-di([1,1′-biphenyl]-4-yl)-9H-carbazole, 0.596 g 1-bromopyrene (2.12 mmol, 2.0 eq), 0.220 g K₂CO₃ (1.59 mmol, 1.5 eq), and 0.0424 g of bronze powder (1 g/25 mmol carbazole) and equipped with a stir bar. The reaction mixture was degassed and placed under a nitrogen atmosphere. The tube was then heated to 220-240° C. for 72 hours to produce a dark green-brown crude mixture. The crude mixture was dissolved in DCM and passed through a silica gel plug with 60:40 hexanes:DCM twice and then recrystallized with layering DCM:MeOH. An orange impurity remained, and the mixture was dry loaded onto silica gel and purified by flash chromatography on silica gel, eluting with a gradient of 0-30% DCM in hexanes. After further purification by another silica gel plug, the product was dissolved in 30 mL DCM and recrystallized by layering 70 mL hexanes. After filtration, 327 mg of a pale yellow solid was recovered (45.9% yield). ¹H NMR (300 MHz, Chloroform-d) δ 8.64-8.56 (m, 2H), 8.41 (d, J=8.1 Hz, 1H), 8.32 (dd, J=7.7, 1.2 Hz, 1H), 8.28-8.21 (m, 3H), 8.17 (d, J=8.1 Hz, 1H), 8.09 (t, J=7.6 Hz, 1H), 8.01 (d, J=9.3 Hz, 1H), 7.91-7.81 (m, 4H), 7.77-7.64 (m, 11H), 7.54-7.43 (m, 4H), 7.42-7.31 (m, 2H), 7.14 (dd, J=8.5, 0.6 Hz, 2H).

### Example 7: Preparation of Acridine PC1

### (a) Synthesis of 9,9-dimethyl-10-(naphthalen-1-yl)-9,10-dihydroacridine

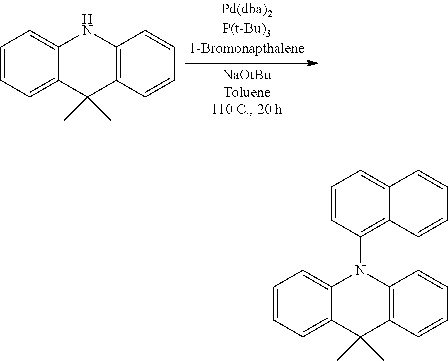 [SMILES: CC1(C)c2ccccc2Nc2ccccc21 ; CC1(C)c2ccccc2N(c2cccc3ccccc23)c2ccccc21]

A storage tube was loaded with 5.0 g (23.9 mmol, 1 eq.) 9,10-dihydro-9,9-dimethylacridine, 7.42 g 1-bromonaphthalene (36.0 mmol, 1.5 eq.), 137.5 mg of bis(dibenzylideneacetone)palladium(0) (0.89 μmol, 1 mol %), 717 μL of 1M in toluene tri-tert-butylphosphine (0.717 mmol, 3 mol %), 6.0 g sodium tert-butoxide (71.7 mmol), and 100 mL toluene under nitrogen atmosphere. The solution was heated to 110° C. After 20 hours, the brown-orange liquid was poured directly through a silica plug and rinsed with toluene. All blue fluorescent portions were collected and concentrated to 50 mL volume via rotary evaporation. The product was precipitated by slow addition of ˜75 mL of ethyl acetate. The product, a white solid, was isolated by vacuum filtration and washed with ethyl acetate and methanol. The product was dried overnight under vacuum to yield 5.88 g (73.4% yield). ¹H NMR (400 MHz, Chloroform-d) δ 8.06-7.95 (m, 2H), 7.68 (ddd, J=8.4, 4.1, 3.0 Hz, 2H), 7.57-7.46 (m, 4H), 7.36 (ddd, J=8.3, 6.8, 1.2 Hz, 1H), 6.96-6.78 (m, 4H), 6.03 (dd, J=8.1, 1.4 Hz, 2H), 1.85 (s, 3H), 1.75 (s, 3H). ¹³C NMR (101 MHz, CDCl₃) δ 140.51, 137.71, 135.47, 131.84, 129.71, 129.04, 128.80, 128.67, 127.10, 126.93, 126.68, 126.54, 125.60, 123.78, 120.47, 114.20, 77.34, 77.02, 76.70, 36.03, 33.02, 31.89. HRMS (ESI) calculated for (M+H)+ for C₂₅H₂₁N, 336.17468; Found, 336.17468.

### (b) Synthesis of 2,7-dibromo-9,9-dimethyl-10-(naphthalen-1-yl)-9,10-dihydroacridine

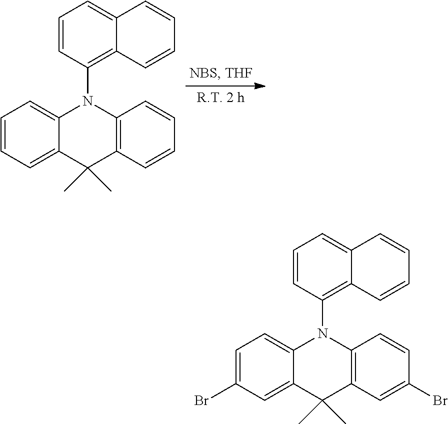 [SMILES: CC1(C)c2ccccc2N(c2cccc3ccccc23)c2ccccc21 ; CC1(C)c2cc(Br)ccc2N(c2cccc3ccccc23)c2ccc(Br)cc21]

5.0 g of 9,10-Dihydro-9,9-dimethyl-10-(1-napthalenyl)-acridine (14.9 mmol, 1.0 eq.) was dissolved in 200 mL THE under ambient atmosphere. 5.83 g of N-bromosuccinimide (32.8 mmol, 2.2 eq.) was slowly added to make a light brown solution. The reaction then stirred for 2 hours. The solution was then concentrated via rotary evaporation, washed with water 3 times, and dried with magnesium sulfate. The product was recrystallized using DCM layered with methanol at −10° C. overnight. The product was isolated by filtration and dried under vacuum to give a pale brown solid, which was used without further purification. Yield: 6.4 g, 87%. ¹H NMR (400 MHz, Chloroform-d) δ 8.08-7.97 (m, 2H), 7.67 (dd, J=8.3, 7.2 Hz, 1H), 7.59-7.50 (m, 4H), 7.47 (dd, J=7.2, 1.2 Hz, 1H), 7.44-7.36 (m, 1H), 6.93 (dd, J=8.8, 2.3 Hz, 2H), 5.90 (d, J=8.8 Hz, 2H), 1.82 (s, 3H), 1.69 (s, 3H). ¹³C NMR (101 MHz, Chloroform-d) δ 139.32, 136.71, 135.50, 131.37, 131.18, 129.54, 129.35, 128.88, 128.72, 128.42, 127.44, 126.94, 126.91, 123.24, 116.04, 113.27, 36.35, 32.87, 31.30.

### (c) Synthesis of 2,7-di([1,1′-biphenyl]-4-yl)-9,9-dimethyl-10-(naphthalen-1-yl)-9,10-dihydroacridine

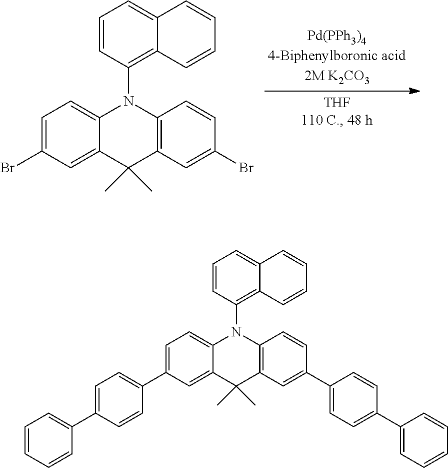 [SMILES: CC1(C)c2cc(Br)ccc2N(c2cccc3ccccc23)c2ccc(Br)cc21 ; CC1(C)c2cc(-c3ccc(-c4ccccc4)cc3)ccc2N(c2cccc3ccccc23)c2ccc(-c3ccc(-c4ccccc4)cc3)cc21]

2.0 g 2,7-Dibromo-9,10-dihydro-9,9-dimethyl-10-(1-napthalenyl)-acridine (4.1 mmol, 1 eq.) was loaded into a storage tube. 3.2 g 4-Biphenylboronic acid (16.2 mmol, 4 eq.) was added under ambient conditions. The flask was brought into a nitrogen-filled glovebox. Then, 0.468 g tetrakis(triphenylphosphine)palladium(0) (0.41 mmol, 10 mol %) was added. 60 mL of THF was added to produce a yellow solution. The flask was taken out of the glovebox, where 45 mL of degassed 2M K₂CO₃ was added using a long needle and syringe. The biphasic solution was then sealed and heated to 110° C. for 48 hours. At that time, the solution was cooled to room temperature and concentrated on rotovap to produce a reddish-brown oil. The crude mixture was redissolved in DCM then passed through a silica plug. The yellow filtrate was collected and concentrated, then purified by column chromatography with hexanes:ethyl acetate ramping from 100:0 to 70:30. The product, a white solid, was then recrystallized with DCM/MeOH at −25° C. to give 1.61 g of a fluffy white solid with 62% yield. ¹H NMR (400 MHz, Chloroform-d) δ 8.12-8.01 (m, 2H), 7.82 (d, J=2.1 Hz, 2H), 7.79-7.71 (m, 2H), 7.68-7.54 (m, 14H), 7.45 (dd, J=8.4, 7.1 Hz, 5H), 7.38-7.30 (m, 2H), 7.16 (dd, J=8.5, 2.1 Hz, 2H), 6.16 (d, J=8.5 Hz, 2H), 2.01 (s, 3H), 1.91 (s, 3H). ¹³C NMR (101 MHz, Chloroform-d) δ 140.84, 140.15, 139.85, 139.28, 137.48, 135.52, 132.98, 131.71, 130.09, 129.05, 128.96, 128.79, 127.44, 127.33, 127.18, 126.98, 126.84, 125.31, 124.62, 123.67, 114.80, 36.44, 33.50, 32.48. HRMS (ESI) calculated for (M+H)+ for C₄₉H₃₇N, 640,29988; Found, 640.29710.

### Example 8: Preparation of Acridine PC2

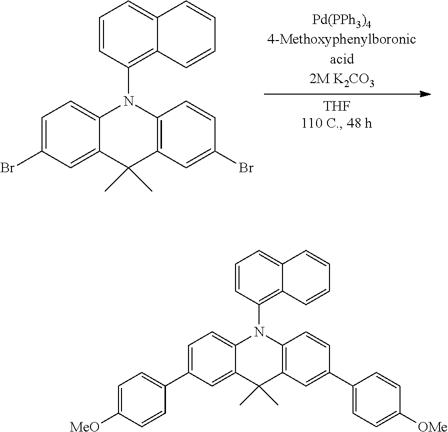 [SMILES: CC1(C)c2cc(Br)ccc2N(c2cccc3ccccc23)c2ccc(Br)cc21 ; COc1ccc(-c2ccc3c(c2)C(C)(C)c2cc(-c4ccc(OC)cc4)ccc2N3c2cccc3ccccc23)cc1]

2.5 g 2,7-Dibromo-9,10-dihydro-9,9-dimethyl-10-(1-napthalenyl)-acridine (5.1 mmol, 1 eq.) was loaded into a storage tube. 3.1 g 4-Methoxyphenylboronic acid (20.2 mmol, 4 eq.) was added under ambient conditions. The flask was brought into a nitrogen-filled glovebox. Then, 0.586 g tetrakis(triphenylphosphine)palladium(0) (0.51 mmol, 10 mol %) was added. 80 mL of THE was added to produce a yellow solution. The flask was taken out of the glovebox, where 56 mL of degassed 2M K₂CO₃ was added using a long needle and syringe. The biphasic solution was then sealed and heated to 110° C. for 24 hours. At that time, the solution was cooled to room temperature and concentrated on rotovap to produce a reddish oil. The crude mixture was dissolved in 200 mL ethyl acetate and washed 3 times with water. The organic layer was dried with magnesium sulfate, filtered, and concentrated. The product was isolated by column chromatography using 80:20 hexane:ethyl acetate. TLC indicated decomposition of the product when using DCM on silica coated plates. Then, the product was recrystallized 3 times using ethyl acetate layered with methanol to yield a white solid. Yield: 1.76 g, 63.3% yield. ¹H NMR (400 MHz, Benzene-d₆) δ 7.97-7.90 (m, 1H), 7.86 (d, J=2.1 Hz, 2H), 7.72 (dd, J=8.2, 3.8 Hz, 2H), 7.54-7.45 (m, 4H), 7.40-7.28 (m, 2H), 7.25-7.19 (m, 1H), 7.11 (td, J=7.6, 6.8, 1.3 Hz, 1H), 7.02 (dd, J=8.5, 2.1 Hz, 2H), 6.92-6.84 (m, 4H), 6.30 (d, J=8.5 Hz, 2H), 3.36 (s, 6H), 1.91 (s, 3H), 1.81 (s, 3H). HRMS (ESI) calculated for M+ for C₃₉H₃₃NO₂, 547.25058; Found, 547.25074.

### Example 9: Preparation of Acridine PC3

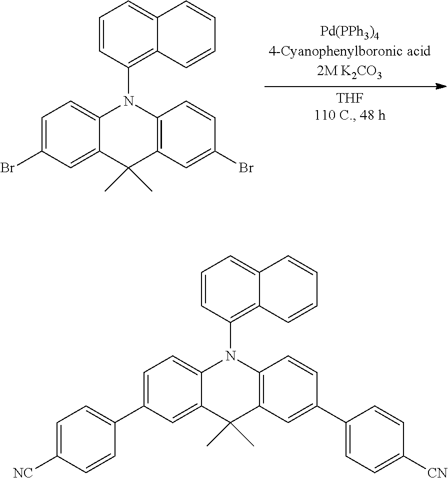 [SMILES: CC1(C)c2cc(Br)ccc2N(c2cccc3ccccc23)c2ccc(Br)cc21 ; CC1(C)c2cc(-c3ccc(C#N)cc3)ccc2N(c2cccc3ccccc23)c2ccc(-c3ccc(C#N)cc3)cc21]

0.3 g of 2,7-Dibromo-9,10-dihydro-9,9-dimethyl-10-(1-napthalenyl)-acridine (0.61 mmol, 1 eq.) and 0.36 g of 4-cyanophenylboronic acid (2.4 mmol, 4 eq.) was loaded into a storage tube under ambient atmosphere. The storage tube was taken into a nitrogen-filled glovebox, then loaded with 0.105 g tetrakis(triphenylphosphine)palladium(0) (0.09 mmol, 15 mol %). The solids were dissolved in 50 mL of THF. The storage tube was sealed and brought out of the glovebox, where 8.0 mL of degassed 2M K₂CO₃ was added using a long needle and syringe to produce a biphasic yellow and colorless solution. The solution was heated to 110° C. for 46 hours, and then brought to room temperature. The solution turned reddish-brown upon exposure to air. The solution was concentrated and extracted into DCM, then passed through a silica plug and rinsed with DCM. The yellow filtrate was collected and concentrated to give a pale-yellow solid. Pure product was obtained by recrystallizing with ethyl acetate layered with methanol at −25° C. to give a yield of 0.287 g, 87%. ¹H NMR (400 MHz, Chloroform-d) δ 8.15-8.05 (m, 2H), 7.82-7.55 (m, 14H), 7.45 (ddd, J=8.3, 6.9, 1.2 Hz, 1H), 7.15 (dd, J=8.6, 2.2 Hz, 2H), 6.19 (d, J=8.6 Hz, 2H), 2.00 (s, 3H), 1.88 (s, 3H). ¹³C NMR (101 MHz, C₆D₆) δ 144.75, 140.56, 137.14, 135.62, 132.27, 132.01, 131.50, 130.19, 129.19, 127.92, 127.68, 127.44, 126.49, 125.87, 124.51, 123.38, 118.70, 115.29, 110.40, 36.22, 33.35, 31.36. HRMS (ESI) calculated for M+ for C₃₉H₂₇N₃, 536.21267; Found, 536.16531.

### Example 10: Preparation of Acridine PC4

### (a) Synthesis of 9,9-dimethyl-10-(naphthalen-2-yl)-9,10-dihydroacridine

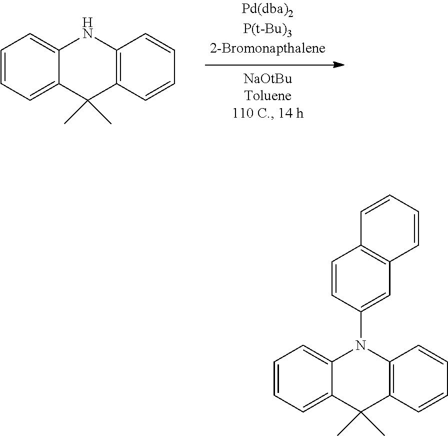 [SMILES: CC1(C)c2ccccc2Nc2ccccc21 ; CC1(C)c2ccccc2N(c2ccc3ccccc3c2)c2ccccc21]

A storage tube was loaded with 1.0 g (4.8 mmol, 1 eq.) 9,10-Dihydro-9,9-dimethylacridine, 1.48 g 2-bromonaphthalene (7.1 mmol, 1.5 eq.), 27.5 mg of bis(dibenzylideneacetone)palladium(0) (0.48 μmol, 1 mol %), 134 μL of 1M in toluene tri-tert-butylphosphine (0.134 mmol, 3 mol %), 1.4 g sodium tert-butoxide (14.4 mmol, 3 eq.), and 50 mL toluene under nitrogen atmosphere. The solution was heated to 110° C. After 14 hours, the reddish-purple liquid with a white precipitate was passed directly through a silica plug and rinsed with toluene. All blue fluorescent portions were collected and concentrated via rotary evaporation. The product was recrystallized 3 times with DCM/methanol at −25° C. The product was collected via vacuum filtration, washed with methanol, and dried overnight under vacuum to yield 1.16 g (72.6% yield). ¹H NMR (400 MHz, Chloroform-d) δ 8.14 (d, J=8.6 Hz, 1H), 8.04-7.98 (m, 1H), 7.91 (dt, J=4.7, 1.8 Hz, 2H), 7.60 (dqd, J=8.3, 6.9, 1.5 Hz, 2H), 7.55-7.48 (m, 2H), 7.44 (dd, J=8.6, 2.0 Hz, 1H), 7.03-6.90 (m, 4H), 6.39-6.27 (m, 2H), 1.77 (s, 6H). ¹³C NMR (101 MHz, Chloroform-d) δ 140.97, 138.49, 134.81, 132.85, 130.95, 130.13, 130.00, 128.93, 128.02, 127.89, 126.73, 126.43, 126.32, 125.22, 120.55, 114.16, 36.02, 31.31. HRMS (ESI) calculated for M+ for C₂₅H₂₁N, 336.17468; Found, 336.1755.

### (b) Synthesis of 2,7-dibromo-9,9-dimethyl-10-(naphthalen-2-yl)-9,10-dihydroacridine

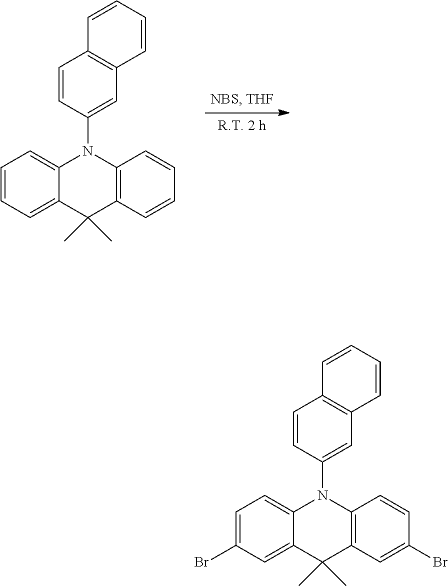 [SMILES: CC1(C)c2ccccc2N(c2ccc3ccccc3c2)c2ccccc21 ; CC1(C)c2cc(Br)ccc2N(c2ccc3ccccc3c2)c2ccc(Br)cc21]

0.75 g of 9,10-Dihydro-9,9-dimethyl-10-(2-napthalenyl)-acridine (2.2 mmol, 1.0 eq.) was dissolved in 20 mL THF under ambient atmosphere. 0.90 g of N-bromosuccinimide (5.0 mmol, 2.25 eq.) was slowly added to make a light brown solution. The reaction then stirred at room temperature for 2 hours. The solution was then concentrated via rotary evaporation, washed with water 3 times, and dried with magnesium sulfate. The product was recrystallized using DCM layered with methanol at −25° C. overnight. The product was isolated by filtration and dried under vacuum to give a pale brown crystalline solid. ¹H NMR revealed a mix of products, which was carried over to the next step without further purification. Yield: 0.95 g, 86%. ¹H NMR (400 MHz, Chloroform-d) δ 8.01 (d, J=8.6 Hz, 1H), 7.93-7.84 (m, 1H), 7.84-7.70 (m, 2H), 7.56-7.39 (m, 5H), 7.31-7.19 (m, 1H), 6.92 (ddd, J=8.8, 4.4, 2.3 Hz, 3H), 6.06 (d, J=8.8 Hz, 2H), 1.58 (s, 7H). ¹³C NMR (101 MHz, CDCl₃) δ 139.83, 137.96, 137.62, 134.73, 132.99, 131.65, 131.57, 131.39, 129.82, 129.57, 129.31, 128.60, 128.08, 128.05, 127.97, 127.83, 127.12, 126.78, 116.01, 115.31, 113.38, 77.33, 77.01, 76.70, 36.34, 31.00, 30.43.

### (c) Synthesis of 2,7-di([1,1′-biphenyl]-4-yl)-9,9-dimethyl-10-(naphthalen-2-yl)-9,10-dihydroacridine

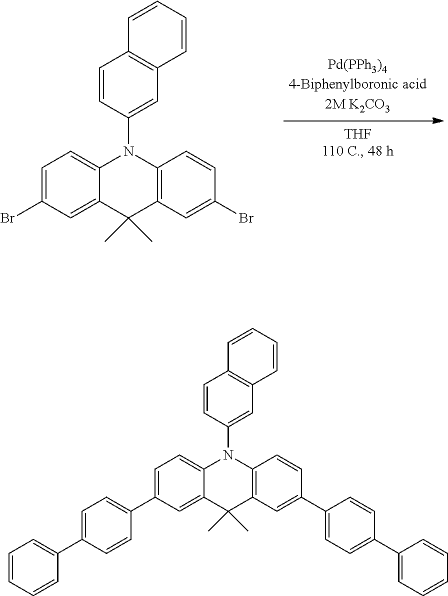 [SMILES: CC1(C)c2cc(Br)ccc2N(c2ccc3ccccc3c2)c2ccc(Br)cc21 ; CC1(C)c2cc(-c3ccc(-c4ccccc4)cc3)ccc2N(c2ccc3ccccc3c2)c2ccc(-c3ccc(-c4ccccc4)cc3)cc21]

0.8 g of 2,7-Dibromo-9,10-dihydro-9,9-dimethyl-10-(2-napthalenyl)-acridine (1.6 mmol, 1 eq.) and 1.28 g of 4-biphenylboronic acid (6.5 mmol, 4 eq.) were loaded into a storage tube under ambient atmosphere. The storage tube was taken into a nitrogen-filled glovebox, then loaded with 0.281 g of tetrakis(triphenylphosphine)palladium(0) (0.243 mmol, 15 mol %). The solids were dissolved in 50 mL THF. The storage tube was sealed and brought out of the glovebox, where 18 mL of degassed 2M K₂CO₃ (22 eq.) was added using a long needle and syringe to produce a biphasic yellow and colorless solution. The solution was heated to 110° C. for 48 hours, and then brought to room temperature. The solution turned reddish-brown upon exposure to air. The solution was concentrated and extracted into DCM, then dried using magnesium sulfate, filtered, and concentrated. The crude mixture was redissolved in DCM then passed through a silica plug. The yellow filtrate was collected and concentrated, then recrystallized with DCM/MeOH at −25° C. to give a white crystalline solid with a yield of 59.4%. ¹H NMR (400 MHz, Chloroform-d) δ 8.17 (d, J=8.6 Hz, 1H), 8.06-8.00 (m, 1H), 7.98-7.90 (m, 2H), 7.79 (d, J=2.1 Hz, 2H), 7.77-7.57 (m, 17H), 7.46 (td, J=8.2, 6.4 Hz, 6H), 7.40-7.29 (m, 3H), 7.23 (d, J=2.1 Hz, 1H), 6.41 (d, J=8.5 Hz, 2H), 1.89 (s, 6H). ¹³C NMR (101 MHz, C₆D₆) δ 141.12, 140.54, 140.52, 139.53, 138.58, 134.97, 133.63, 133.06, 131.12, 130.50, 130.14, 128.79, 127.92, 127.68, 127.44, 125.41, 124.26, 115.15, 36.38, 31.60. HRMS (ESI) calculated for (M+H)+ for C₄₉H₃₇N, 640,29988; Found, 640.2991.

### Example 11: Preparation of Acridine PC5

### (a) Synthesis of 9,9-dimethyl-10-phenyl-9,10-dihydroacridine

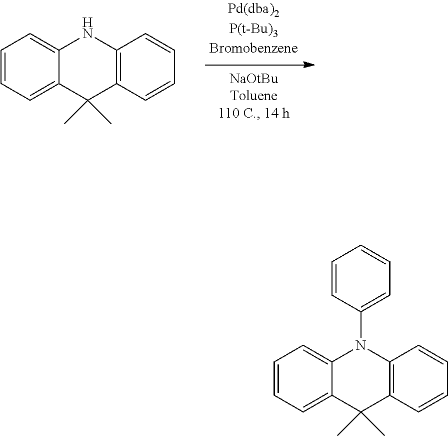 [SMILES: CC1(C)c2ccccc2Nc2ccccc21 ; CC1(C)c2ccccc2N(c2ccccc2)c2ccccc21]

A storage tube was loaded with 1.0 g (4.8 mmol, 1 eq.) 9,10-dihydro-9,9-dimethylacridine, 1.48 g bromobenzene (7.2 mmol, 1.5 eq.), 27.5 mg of bis(dibenzylideneacetone)palladium(0) (0.48 μmol, 1 mol %), 143 μL of 1M in toluene tri-tert-butylphosphine (0.134 mmol, 3 mol %), 1.4 g sodium tert-butoxide (14.4 mmol, 3 eq.), and 27 mL toluene under nitrogen atmosphere. The solution was heated to 110° C. After 14 hours, the reddish liquid with a white precipitate was passed directly through a silica plug and rinsed with toluene. All blue fluorescent portions were collected and concentrated via rotary evaporation. The product was recrystallized with ethyl acetate/methanol at −25° C. for 4 hours. The product was collected via vacuum filtration, washed with methanol, and dried overnight under vacuum to yield 1.0 g (73.5% yield) of a white crystalline solid. ¹H NMR (400 MHz, Chloroform-d) δ 7.67-7.59 (m, 2H), 7.54-7.47 (m, 1H), 7.46 (dd, J=7.5, 1.8 Hz, 2H), 7.37-7.30 (m, 2H), 7.01-6.87 (m, 4H), 6.26 (dd, J=7.9, 1.5 Hz, 2H), 1.70 (s, 6H). ¹³C NMR (101 MHz, Chloroform-d) δ 141.20, 140.93, 131.32, 130.83, 129.95, 128.19, 126.32, 125.17, 120.48, 114.01, 35.98, 31.24. HRMS (ESI) calculated for (M+H)+ for C₂₁H₁₉N, 285.15175; Found, 285.1514.

### (b) Synthesis of 2,7-Dibromo-9,9-dimethyl-10-phenyl-9,10-dihydroacridine

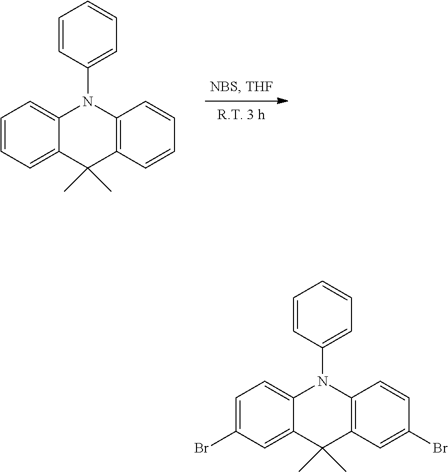 [SMILES: CC1(C)c2ccccc2N(c2ccccc2)c2ccccc21 ; CC1(C)c2cc(Br)ccc2N(c2ccccc2)c2ccc(Br)cc21]

0.75 g of 9,10-Dihydro-9,9-dimethyl-10-(phenyl)-acridine (2.2 mmol, 1.0 eq.) was dissolved in 50 mL THE under ambient atmosphere. 1.16 g of N-bromosuccinimide (5.0 mmol, 2.5 eq.) was slowly added to make a light brown solution. The reaction then stirred at room temperature for 3 hours. The solution was then concentrated via rotary evaporation, washed with water 3 times, and dried with magnesium sulfate. The product was recrystallized using DCM layered with methanol at −25° C. overnight. The product was isolated by filtration and dried under vacuum to give a white solid. ¹H NMR analysis revealed a mix of brominated substitutions, which was carried over to the next step without further purification. Yield: 0.78 g, 67%. ¹H NMR (400 MHz, Chloroform-d) δ 7.71-7.63 (m, 1H), 7.59-7.48 (m, 1H), 7.48-7.37 (m, 2H), 7.22-7.17 (m, 1H), 7.12-7.04 (m, 1H), 6.96 (ddd, J=8.9, 7.9, 2.3 Hz, 2H), 6.02 (dd, J=8.8, 3.0 Hz, 2H), 1.54 (d, J=4.5 Hz, 6H). ¹³C NMR (101 MHz, CDCl₃) δ 140.39, 139.76, 139.45, 139.42, 134.52, 132.70, 131.73, 131.60, 131.12, 130.81, 129.39, 129.29, 128.72, 128.19, 128.02, 122.71, 115.85, 115.70, 113.64, 113.29, 77.33, 77.01, 76.70, 36.28, 30.93.

### (c) Synthesis of 2,7-di([1,1′-biphenyl]-4-yl)-9,9-dimethyl-10-phenyl-9,10-dihydroacridine

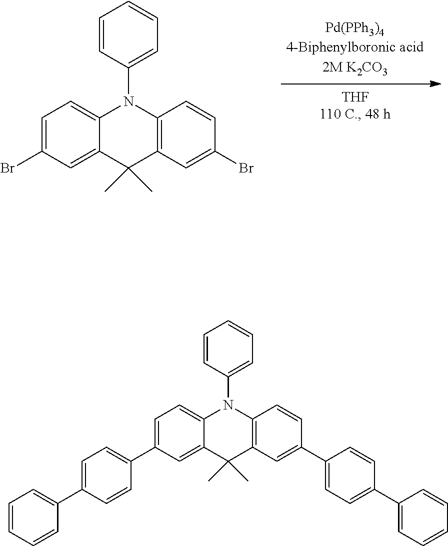 [SMILES: CC1(C)c2cc(Br)ccc2N(c2ccccc2)c2ccc(Br)cc21 ; CC1(C)c2cc(-c3ccc(-c4ccccc4)cc3)ccc2N(c2ccccc2)c2ccc(-c3ccc(-c4ccccc4)cc3)cc21]

0.5 g of 2,7-Dibromo-9,10-dihydro-9,9-dimethyl-10-(phenyl)-acridine (1.1 mmol, 1 eq.) and 0.89 g of 4-biphenylboronic acid (4.5 mmol, 4 eq.) was loaded into a storage tube under ambient atmosphere. The storage tube was taken into a nitrogen-filled glovebox, then loaded with 0.194 g tetrakis(triphenylphosphine)palladium(0) (0.168 mmol, 15 mol %). The solids were dissolved in 20 mL THF. The storage tube was sealed and brought out of the glovebox, where 12 mL of degassed 2M K₂CO₃ (22 eq.) was added using a long needle and syringe to produce a biphasic yellow and colorless solution. The solution was heated to 110° C. for 48 hours, then brought to room temperature. The solution turned reddish-brown upon exposure to air. The solution was concentrated and extracted into DCM, then dried using magnesium sulfate, filtered, and concentrated. The crude mixture was redissolved in DCM then passed through a silica plug. The yellow filtrate was collected and concentrated, then purified by column chromatography with hexanes:ethyl acetate ramping from 100:0 to 70:30. The product, a white solid, was then recrystallized with Ethyl acetate/MeOH at −25° C. to give 0.665 g of a white crystalline solid with a yield of 62%. ¹H NMR (400 MHz, Benzene-d₆) δ 7.94 (d, J=2.1 Hz, 2H), 7.74-7.54 (m, 19H), 7.40-7.25 (m, 10H), 6.68 (d, J=8.5 Hz, 2H), 1.83 (s, 6H). ¹³C NMR (101 MHz, CDCl₃) δ 141.01, 140.86, 140.21, 139.33, 133.12, 133.03, 131.52, 131.16, 131.00, 130.36, 130.27, 129.52, 128.89, 128.83, 128.80, 128.46, 127.69, 127.59, 127.56, 127.49, 127.47, 127.38, 127.19, 127.08, 127.05, 126.99, 126.90, 126.89, 125.09, 124.23, 114.61, 77.34, 77.02, 76.70, 36.38, 31.85. HRMS (ESI) calculated for (M+H)+ for C₄₅H₃₅N, 589.27694; Found, 589.2764.

### Example 12: Preparation of Acridine PC6

### (a) Synthesis of 10-(4-methoxyphenyl)-9,9-dimethyl-9,10-dihydroacridine

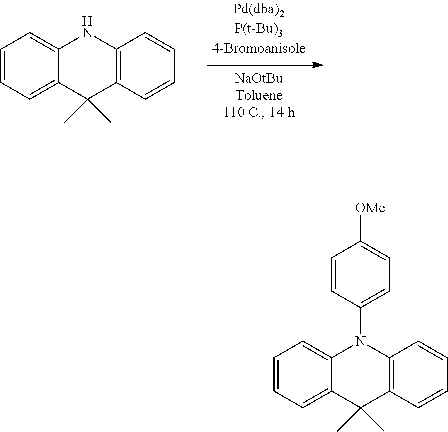 [SMILES: CC1(C)c2ccccc2Nc2ccccc21 ; COc1ccc(N2c3ccccc3C(C)(C)c3ccccc32)cc1]

A storage tube was loaded with 1.0 g (4.8 mmol, 1 eq.) 9,10-dihydro-9,9-dimethylacridine, 0.9 mL 4-bromoanisole (7.2 mmol, 1.5 eq.), 27.5 mg of bis(dibenzylideneacetone)palladium(0) (0.48 μmol, 1 mol %), 143 μL of 1M in toluene tri-tert-butylphosphine (0.134 mmol, 3 mol %), 1.4 g sodium tert-butoxide (14.4 mmol, 3 eq.), and 27 mL toluene under nitrogen atmosphere. The solution was heated to 110° C. After 14 hours, the brown liquid with a white precipitate was passed directly through a silica plug and rinsed with toluene. All fluorescent blue portions were collected and concentrated via rotary evaporation. The product was recrystallized with DCM/methanol at −25° C. for 2 hours. The product was collected via vacuum filtration, washed with methanol, and dried overnight under vacuum to yield 1.25 g (82.8% yield) of white crystalline needles. ¹H NMR (400 MHz, Benzene-d₆) δ 7.45-7.36 (m, 2H), 7.02-6.88 (m, 6H), 6.82-6.73 (m, 2H), 6.56-6.44 (m, 2H), 3.29 (s, 3H), 1.65 (s, 6H). ¹³C NMR (101 MHz, C₆D₆) δ 159.16, 141.52, 133.83, 132.15, 129.97, 126.43, 125.22, 120.62, 115.89, 114.28, 54.61, 35.89, 31.09. HRMS (ESI) calculated for (M+H)+ for C₂₂H₂₁NO, 316.16959; Found, 316.16916.

### (b) Synthesis of 2,7-Dibromo-10-(4-methoxyphenyl)-9,9-dimethyl-9,10-dihydroacridine

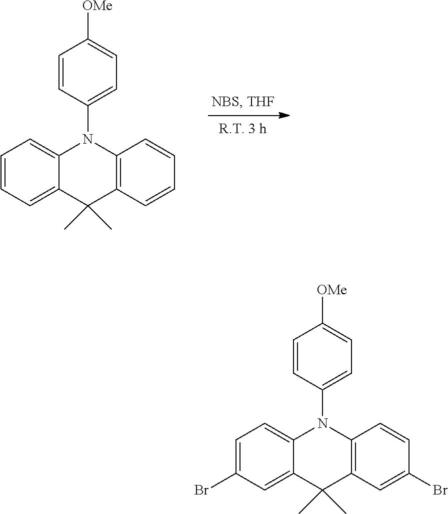 [SMILES: COc1ccc(N2c3ccccc3C(C)(C)c3ccccc32)cc1 ; COc1ccc(N2c3ccc(Br)cc3C(C)(C)c3cc(Br)ccc32)cc1]

1.0 g of methoxyphenyl)-acridine (3.2 mmol, 1.0 eq.) was dissolved in 50 mL THF under ambient atmosphere. 1.41 g of N-bromosuccinimide (7.9 mmol, 2.5 eq.) was slowly added to make a light brown solution. The reaction then stirred at room temperature for 3 hours. The solution was then concentrated via rotary evaporation, washed with water 3 times, and dried with magnesium sulfate. The product was recrystallized using DCM layered with methanol at −25° C. overnight. The product was isolated by filtration and dried under vacuum to give a brownish-white solid, which was used without further purification. Yield: 1.16 g, 77.6%. ¹H NMR (400 MHz, Chloroform-d) δ 7.49 (d, J=2.3 Hz, 2H), 7.21-7.09 (m, 4H), 7.05 (dd, J=8.8, 2.3 Hz, 2H), 6.17 (d, J=8.7 Hz, 2H), 3.91 (s, 3H), 1.63 (s, 6H). ¹³C NMR (101 MHz, Chloroform-d) δ 159.46, 140.08, 132.78, 131.71, 131.60, 129.27, 127.97, 116.22, 115.86, 113.17, 55.56, 36.26, 30.93.

### (c) Synthesis of 2,7-Di([1,1′-biphenyl]-4-yl)-10-(4-methoxyphenyl)-9,9-dimethyl-9,10-dihydroacridine

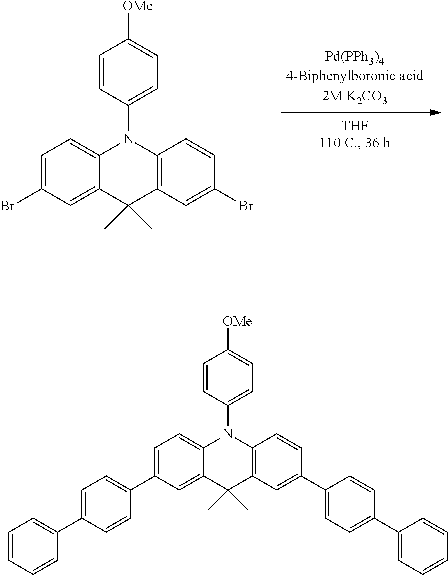 [SMILES: COc1ccc(N2c3ccc(Br)cc3C(C)(C)c3cc(Br)ccc32)cc1 ; COc1ccc(N2c3ccc(-c4ccc(-c5ccccc5)cc4)cc3C(C)(C)c3cc(-c4ccc(-c5ccccc5)cc4)ccc32)cc1]

0.5 g of 2,7-Dibromo-9,10-dihydro-9,9-dimethyl-10-(4-methoxyphenyl)-acridine (1.1 mmol, 1 eq.) and 0.89 g of 4-biphenylboronic acid (4.5 mmol, 4 eq.) was loaded into a storage tube under ambient atmosphere. The storage tube was taken into a nitrogen-filled glovebox, then loaded with 0.184 g of tetrakis(triphenylphosphine)palladium(0) (0.159 mmol, 15 mol %). The solids were dissolved in 40 mL THF. The storage tube was sealed and brought out of the glovebox, where 12 mL of degassed 2M K₂CO₃ (22 eq.) was added using a long needle and syringe to produce a biphasic yellow and colorless solution. The solution was heated to 110° C. for 36 hours, then brought to room temperature. The solution turned reddish-brown upon exposure to air. The solution was concentrated and extracted into DCM, then dried using magnesium sulfate, filtered, and concentrated. The crude mixture was redissolved in DCM then passed through a silica plug. The filtrate was concentrated, then dissolved in toluene and passed through an additional silica plug to remove residual palladium. The filtrate was collected and concentrated, then purified by column chromatography with hexanes:ethyl acetate ramping from 100:0 to 80:20. The product, a white solid, was then recrystallized with ethyl acetate/MeOH at −25° C. to give 0.385 g of a white solid with a yield of 58.6%. ¹H NMR (400 MHz, Benzene-d₆) δ 7.91 (d, J=2.1 Hz, 2H), 7.73-7.65 (m, 4H), 7.64-7.50 (m, 11H), 7.40-7.33 (m, 3H), 7.27 (td, J=7.7, 2.4 Hz, 5H), 6.91-6.84 (m, 2H), 6.64 (d, J=8.5 Hz, 2H), 3.34 (s, 3H), 1.81 (s, 6H). ¹³C NMR (101 MHz, CDCl₃) δ 159.32, 140.86, 140.68, 140.53, 140.24, 139.58, 139.30, 132.93, 132.02, 130.27, 128.83, 128.79, 127.56, 127.46, 127.38, 127.18, 127.05, 126.99, 126.88, 125.08, 124.21, 116.12, 114.61, 77.33, 77.01, 76.70, 55.59, 36.34, 31.86. HRMS (ESI) calculated for (M+H)⁺ for C₄₆H₃₇NO, 619.28751; Found, 619.2877.

### Example 13: Preparation of Acridine PC7

### (a) Synthesis of 4-(9,9-Dimethylacridin-10(9H)-yl)benzonitrile

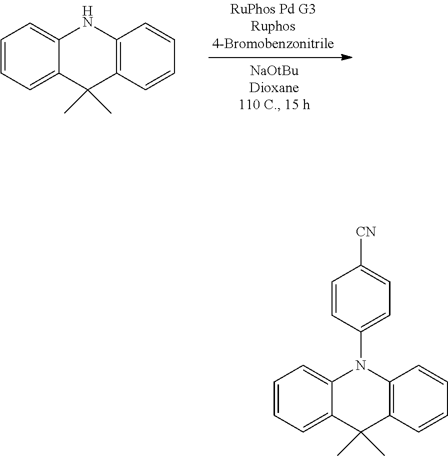 [SMILES: CC1(C)c2ccccc2Nc2ccccc21 ; CC1(C)c2ccccc2N(c2ccc(C#N)cc2)c2ccccc21]

A storage tube was loaded with 2.0 g (9.6 mmol, 1 eq.) 9,10-dihydro-9,9-dimethylacridine, 3.5 g 4-bromobenzonitrile (19.1 mmol, 2.0 eq.), 0.118 g RuPhos ligand (0.28 mmol, 0.03 eq.), and 1.8 g sodium tert-butoxide (19.1 mmol, 2 eq.) under ambient atmosphere, then brought into a nitrogen-filled glovebox. Then, 0.244 g RuPhos Pd G3 precatalyst (0.28 mmol, 0.03 eq.) and 15 mL of degassed dioxane was added. The solution was sealed and brought outside of the glovebox, then heated to 110° C. for 15 hours. The solution was cooled to room temperature, then transferred to a flask and concentrated. The brown solid was dissolved in toluene then passed through a silica plug. The filtrate was concentrated, then recrystallized with DCM/MeOH at −25° C. overnight. The product was collected via vacuum filtration, washed with methanol, and dried overnight under vacuum to yield 1.7 g (57.3% yield) of pale-yellow solid. ¹H NMR (400 MHz, Chloroform-d) δ 8.21-8.12 (m, 2H), 7.79-7.70 (m, 4H), 7.32-7.19 (m, 4H), 6.59-6.51 (m, 2H), 1.93 (s, 6H). ¹³C NMR (101 MHz, Chloroform-d) δ 146.12, 140.17, 134.76, 131.53, 131.06, 126.48, 125.45, 121.69, 118.39, 114.81, 111.38, 36.21, 30.85. HRMS (ESI) calculated for (M+H)⁺ for C₂₂H₁₈N₂, 311.15428; Found, 311.15351.

### (b) Synthesis of 4-(2,7-Dibromo-9,9-dimethylacridin-10(9H)-yl)benzonitrile

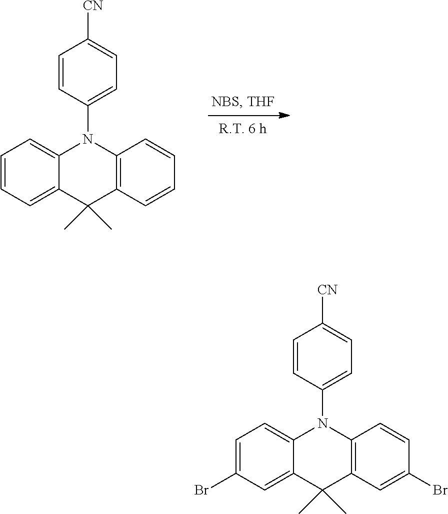 [SMILES: CC1(C)c2ccccc2N(c2ccc(C#N)cc2)c2ccccc21 ; CC1(C)c2cc(Br)ccc2N(c2ccc(C#N)cc2)c2ccc(Br)cc21]

1.0 g of 9,10-Dihydro-9,9-dimethyl-10-(4-cyanophenyl)-acridine (3.2 mmol, 1.0 eq.) was dissolved in 20 mL THF under ambient atmosphere. 1.26 g of N-bromosuccinimide (7.1 mmol, 2.2 eq.) was slowly added to make a colorless solution. The reaction then stirred at room temperature for 6 hours. The solution was then concentrated via rotary evaporation, washed with water 3 times, and dried with magnesium sulfate. The product was recrystallized using DCM layered with methanol at −25° C. overnight. The product was isolated by filtration and dried under vacuum to give a pale yellow crystalline solid, which was used without further purification. Yield: 1.0 g, 66%. ¹H NMR (400 MHz, Chloroform-d) δ 7.91-7.82 (m, 2H), 7.46 (d, J=2.3 Hz, 2H), 7.41-7.35 (m, 2H), 7.02 (dd, J=8.8, 2.3 Hz, 2H), 6.01 (d, J=8.8 Hz, 2H), 1.57 (s, 6H). ¹³C NMR (101 MHz, Chloroform-d) δ 145.00, 138.97, 135.07, 132.42, 131.65, 129.50, 128.41, 117.97, 115.93, 114.34, 112.59, 36.40, 30.78.

### (c) Synthesis of 4-(2,7-Di([1,1′-biphenyl]-4-yl)-9,9-dimethylacridin-10(9H)-yl)benzonitrile

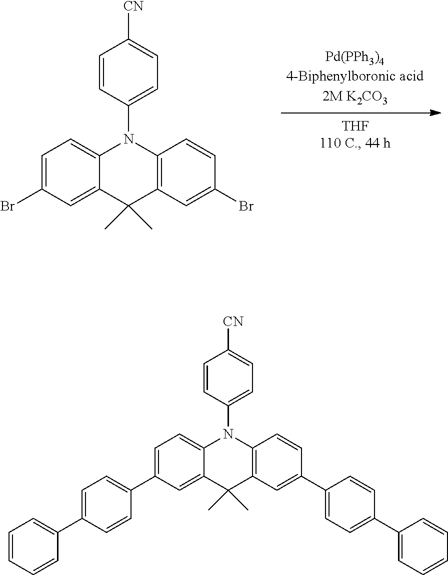 [SMILES: CC1(C)c2cc(Br)ccc2N(c2ccc(C#N)cc2)c2ccc(Br)cc21 ; CC1(C)c2cc(-c3ccc(-c4ccccc4)cc3)ccc2N(c2ccc(C#N)cc2)c2ccc(-c3ccc(-c4ccccc4)cc3)cc21]

0.8 g of 2,7-Dibromo-9,10-dihydro-9,9-dimethyl-10-(4-cyanophenyl)-acridine (1.7 mmol, 1 eq.) and 1.4 g of 4-biphenylboronic acid (6.8 mmol, 4 eq.) was loaded into a storage tube under ambient atmosphere. The storage tube was taken into a nitrogen-filled glovebox, then loaded with 0.296 g of tetrakis(triphenylphosphine)palladium(0) (0.257 mmol, 15 mol %). The solids were dissolved in 50 mL degassed THF. The storage tube was sealed and brought out of the glovebox, where 19 mL of degassed 2M K₂CO₃ (22 eq.) was added using a long needle and syringe to produce a biphasic yellow and colorless solution. The solution was heated to 110° C. for 44 hours, then brought to room temperature. The solution turned red upon exposure to air. The solution was concentrated and extracted into DCM. A yellow emulsion formed, which was filtered and washed with methanol to produce the crude product as a pale-yellow powder. The solid was dissolved in DCM and washed with water 3 times, then dried with magnesium sulfate and concentrated. The product was recrystallized with DCM/MeOH at room temperature 2 times to give 0.88 g at 84% yield. ¹H NMR (400 MHz, Benzene-d₆) δ 7.87 (d, J=2.1 Hz, 2H), 7.72-7.55 (m, 12H), 7.38-7.24 (m, 6H), 7.24-7.18 (m, 2H), 7.11-7.03 (m, 2H), 6.84-6.75 (m, 2H), 6.23 (d, J=8.5 Hz, 2H), 1.73 (s, 6H). ¹³C NMR (101 MHz, CDCl₃) δ 145.01, 141.04, 140.22, 139.99, 139.64, 134.51, 134.47, 131.34, 131.25, 128.92, 128.00, 127.76, 127.52, 127.18, 127.10, 125.39, 124.46, 118.01, 115.07, 112.21, 36.42, 31.27. HRMS (ESI) calculated for (M+H)⁺ for C₄₆H₃₄N₂, 614.2722; Found, 614.2736.

### UV-Vis Absorption

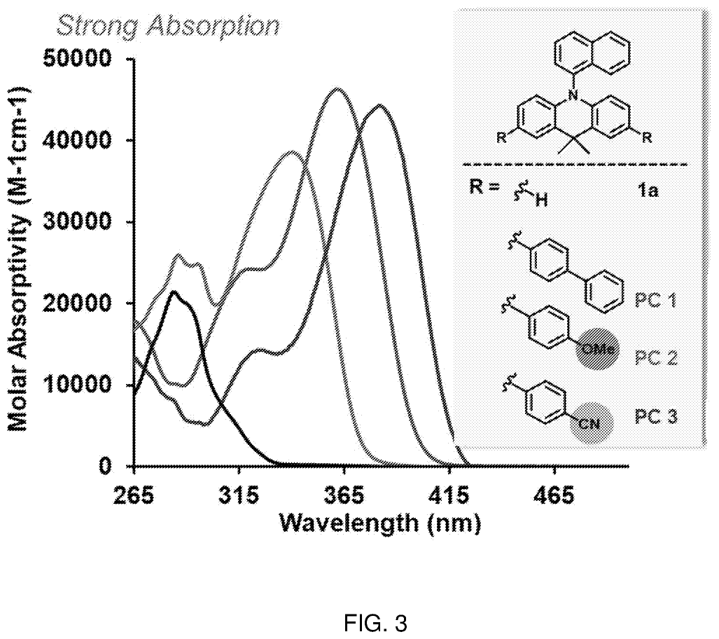

Characteristic UV-vis absorption spectra were obtained for selective carbazole and acridine PCs. FIG. 2 shows the UV-vis absorption spectra for selective carbazole PCs. FIG. 3 shows the characteristic UV-vis absorption spectra for selective acridine PCs.

### Solvatochromism

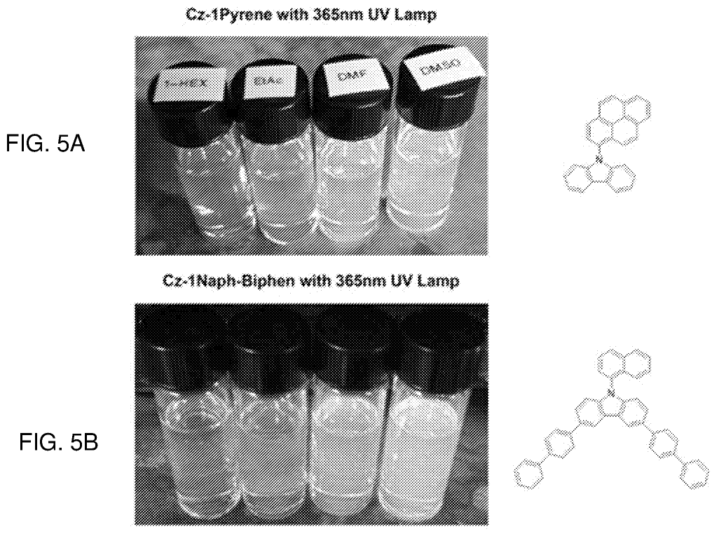

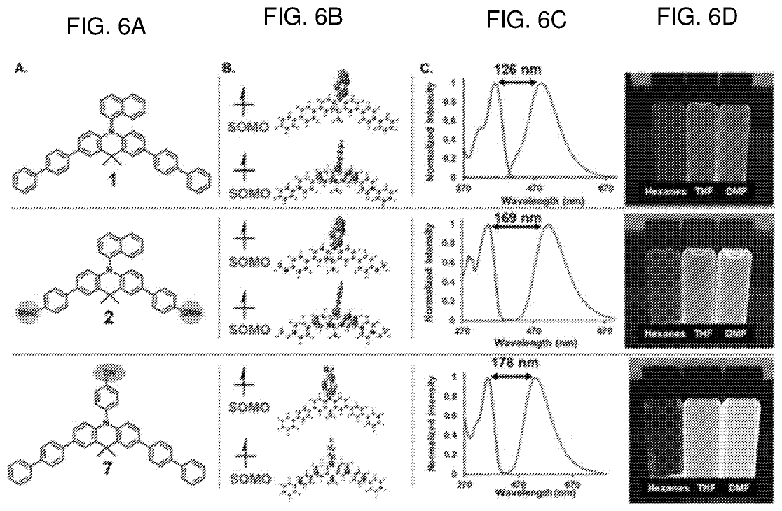

Solvatochromism of selective carbazoles and acridine PCs are shown in FIGS. 5A-5B and FIGS. 6A-6D using a 365 nm UV lamp.

### Computational Details

All calculations were performed using computational chemistry software package Gaussian 09 ver. D01. The use of a Comet supercomputer was used in these calculations.

(a) Computational Predictions and Characterization of Carbazole Catalysts

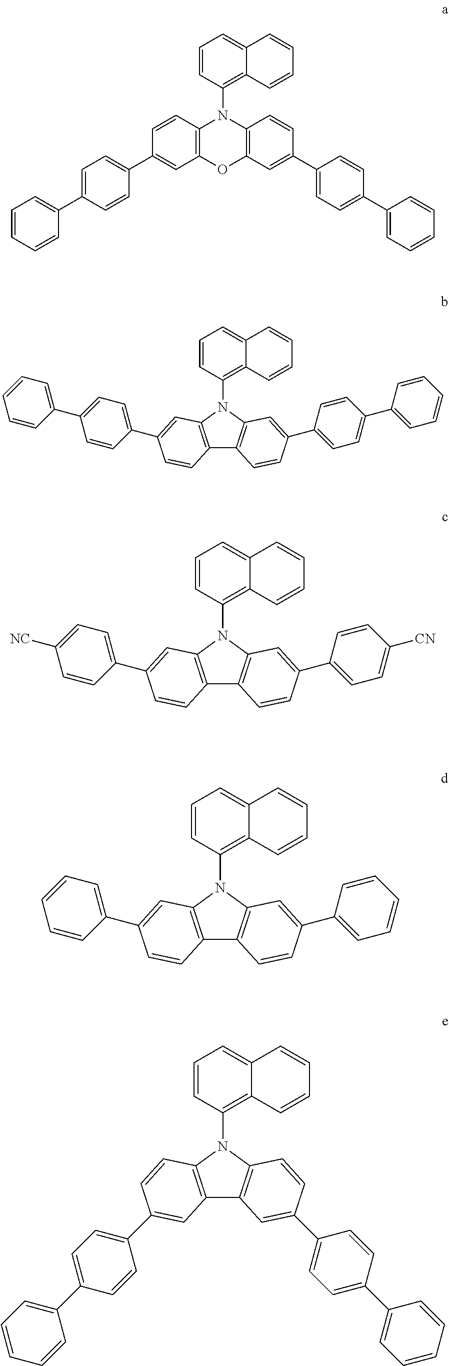 [SMILES: c1ccc(-c2ccc(-c3ccc4c(c3)Oc3cc(-c5ccc(-c6ccccc6)cc5)ccc3N4c3cccc4ccccc34)cc2)cc1 ; c1ccc(-c2ccc(-c3ccc4c5ccc(-c6ccc(-c7ccccc7)cc6)cc5n(-c5cccc6ccccc56)c4c3)cc2)cc1 ; N#Cc1ccc(-c2ccc3c4ccc(-c5ccc(C#N)cc5)cc4n(-c4cccc5ccccc45)c3c2)cc1 ; c1ccc(-c2ccc3c4ccc(-c5ccccc5)cc4n(-c4cccc5ccccc45)c3c2)cc1 ; c1ccc(-c2ccc(-c3ccc4c(c3)c3cc(-c5ccc(-c6ccccc6)cc5)ccc3n4-c3cccc4ccccc34)cc2)cc1]

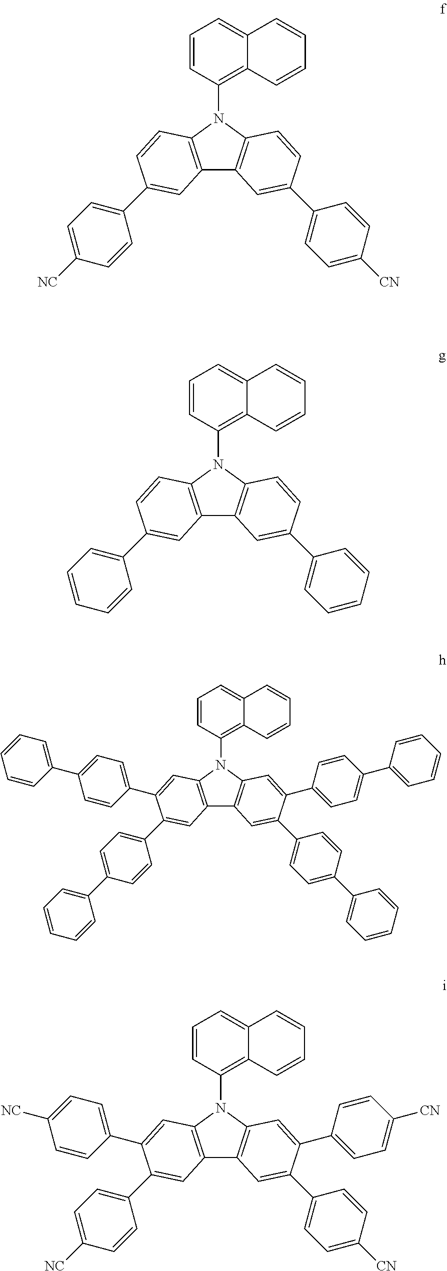

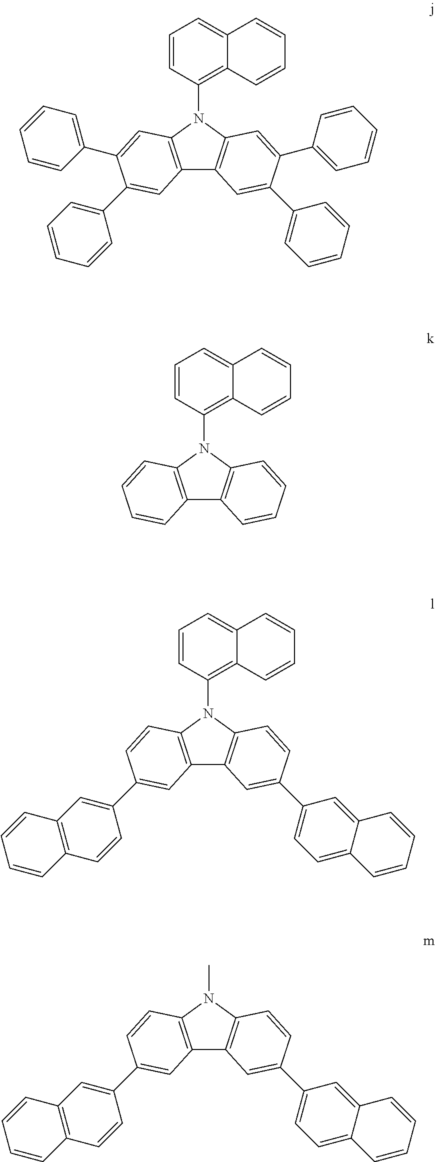

Summary of the computationally derived characteristics for carbazole catalysts are shown in the below table.

TABLE 1

| | | Triplet | E⁰*_{T1, calc} | E⁰ₒₓ | T1 −> S0 | | |
|---|---|---|---|---|---|---|---|
| | | energy | (²PC^{·+}/³PC), | (²PC^{·+}/¹PC), | wavelength | λ_{max, abs} | |
| | PC | (eV) | V vs. SCE | V vs. SCE | (nm) | (nm) | f |
| a) | PhenO_1Naph_Biphen_neutral_triplet | 2.11 | −1.70 | 0.42 | 586 | 351.27 | 0.8443 |
| b) | Carbazole_1Naph_27- | 2.40 | −1.47 | 0.93 | 518 | 325.10 | 2.4569 |
| | Biphen_neutral_triplet | | | | | | |
| c) | Carbazole_1Naph_27- | 2.39 | −1.11 | 1.29 | 518 | 326.36 | 1.9349 |
| | PhCN_neutral_triplet | | | | | | |
| d) | Carbazole_1Naph_27- | 2.39 | −1.22 | 1.17 | 518 | 308.41 | 1.2811 |
| | PhH_neutral_triplet | | | | | | |
| e) | Carbazole_1Naph_36- | 2.37 | −1.44 | 0.92 | 524 | 303.38 | 1.6644 |
| | Biphen_neutral_triplet | | | | | | |
| f) | Carbazole_1Naph_36- | 2.39 | −1.22 | 1.17 | 518 | 312.92 | 1.2161 |
| | PhenCN_neutral_triplet | | | | | | |
| g) | Carbazole_1Naph_36- | 2.37 | −1.42 | 0.94 | 523 | 297.35 | 0.2730 |
| | PhH_neutral_triplet | | | | | | |
| h) | Carbazole_1Naph_2367- | 2.35 | −1.37 | 0.98 | 527 | 313.06 | 1.7846 |
| | Biphen_neutral_triplet | | | | | | |
| i) | Carbazole_1Naph_2367- | 2.37 | −1.09 | 1.28 | 523 | 312.35 | 0.6621 |
| | PhCN_neutral_triplet | | | | | | |
| j) | Carbazole_1Naph_2367- | 2.38 | −1.30 | 1.08 | 520 | 299.65 | 0.9726 |
| | PhH_neutral_triplet | | | | | | |
| k) | Carbazole_1Naph_neutral_triplet | 2.39 | −1.25 | 1.14 | 519 | 289.85 | 0.2400 |
| l) | Carbazole_1Naph_2Naph_neutral_triplet | 2.33 | −1.40 | 0.93 | 532 | 302.18 | 0.7006 |
| m) | Carbazole_Me_2Naph_neutral_triplet | 2.33 | −1.44 | 0.89 | 533 | 305.62 | 0.863 |

(b) Computational Predictions and Characterization of Acridine Catalysts

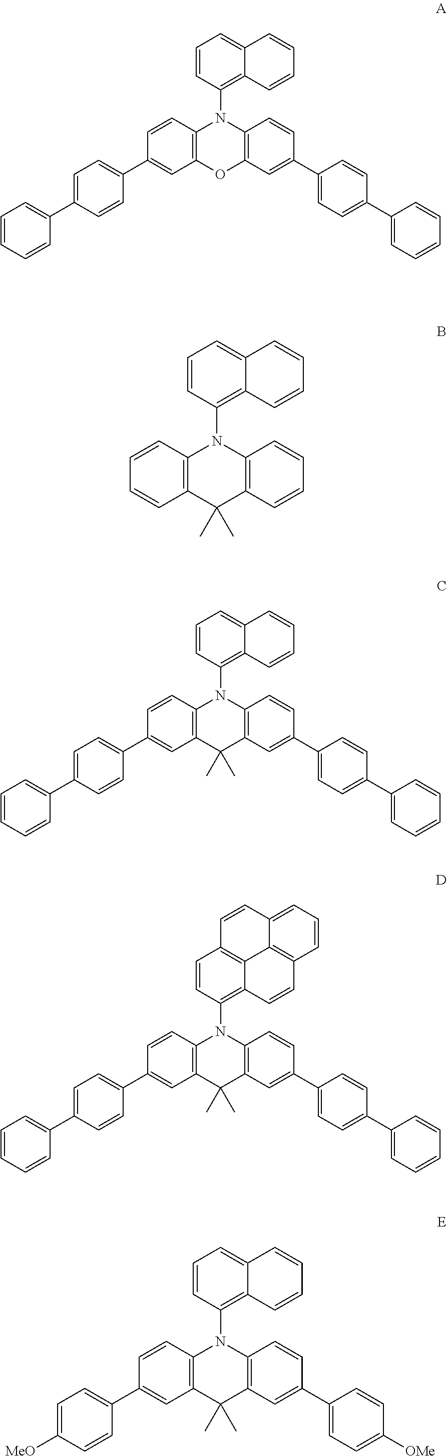 [SMILES: c1ccc(-c2ccc(-c3ccc4c(c3)Oc3cc(-c5ccc(-c6ccccc6)cc5)ccc3N4c3cccc4ccccc34)cc2)cc1 ; CC1(C)c2ccccc2N(c2cccc3ccccc23)c2ccccc21 ; CC1(C)c2cc(-c3ccc(-c4ccccc4)cc3)ccc2N(c2cccc3ccccc23)c2ccc(-c3ccc(-c4ccccc4)cc3)cc21 ; CC1(C)c2cc(-c3ccc(-c4ccccc4)cc3)ccc2N(c2ccc3ccc4cccc5ccc2c3c45)c2ccc(-c3ccc(-c4ccccc4)cc3)cc21 ; COc1ccc(-c2ccc3c(c2)C(C)(C)c2cc(-c4ccc(OC)cc4)ccc2N3c2cccc3ccccc23)cc1]

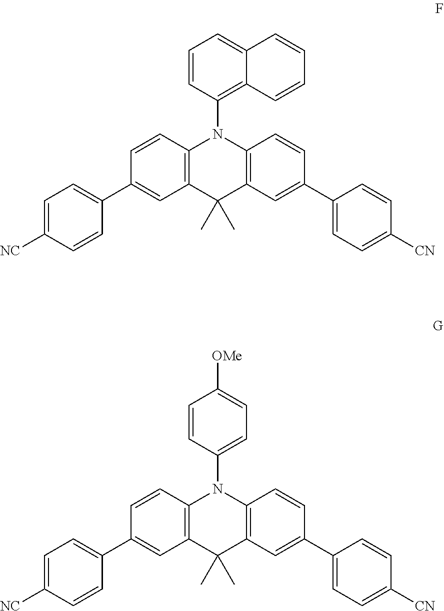 [SMILES: CC1(C)c2cc(-c3ccc(C#N)cc3)ccc2N(c2cccc3ccccc23)c2ccc(-c3ccc(C#N)cc3)cc21 ; COc1ccc(N2c3ccc(-c4ccc(C#N)cc4)cc3C(C)(C)c3cc(-c4ccc(C#N)cc4)ccc32)cc1]

Table 2 shows the summary of computationally derived properties of acridine PCs.

TABLE 2

| | | Triplet | E⁰*_{T1, calc} | E⁰ₒₓ | | |
|---|---|---|---|---|---|---|
| | | energy | (²PC^{·+}/³PC), | (²PC^{·+}/¹PC), | λ_{max, abs} | |
| | PCs | (eV) | V vs. SCE | V vs. SCE | (nm) | f |
| a) | PhenO_1Naph_Biphen_neutral_triplet | 2.11 | −1.70 | 0.42 | 351.27 | 0.8443 |
| b) | Acrid-1Naph_neutral_triplet | 2.38 | −1.66 | 0.72 | 284 | 0.170 |
| c) | Acrid-1Naph-Biphen_neutral_triplet | 2.35 | −1.80 | 0.55 | 326 | 1.689 |
| d) | Acrid-1Pyr-Biphen_neutral_triplet | 1.90 | −1.30 | 0.60 | 327 | 1.732 |
| e) | Acrid-1Naph-OMe_neutral_triplet | 2.36 | −1.94 | 0.42 | 311 | 1.211 |
| f) | Acrid-1Naph-CN_neutral_triplet | 2.43 | −1.69 | 0.74 | 343 | 1.446 |
| g) | Acrid-OMe-CN_neutral_triplet | 2.27 | −1.57 | 0.70 | 347 | 1.452 |

(c) Reduction Potentials

Standard reduction potentials (E⁰) were calculated following previously reported procedures. (He, H.; Zapol, P.; Curtiss, L. A. The Journal of Physical Chemistry C 2010, 114, 21474. Tossell, J. A. Computational and Theoretical Chemistry 2011, 977, 123. Winget, P.; Cramer, C. J.; Truhlar, D. G. Theoretical Chemistry Accounts: Theory, Computation, and Modeling (Theoretica Chimica Acta) 2004, 112, 217. Zhao, Y.; Truhlar, D. Theoretical Chemistry Accounts 2008, 120, 215. A value of −100.5 kcal/mol was assumed for the reduction free energy of the standard hydrogen electrode (SHE). Thus, E⁰=(−100.5−ΔG_{red})/23.06 (V vs. SHE); for E0 (²PC^{●+}/³PC*), ΔG_{red}=G(³PC*)−G(²PC^{●+}) while for E⁰ (²PC^{●+}/¹PC), ΔG_{red}=G(¹PC)−G(²PC^{●+}).

The Gibbs free energies of ³PC*, ²PC^{●+}, and ¹PC were calculated at the unrestricted M06/6-311+G** level of theory in CPCM-H2O solvent (single point energy) using geometries optimized at unrestricted M06/6-31+G** level of theory in CPCM-H₂O solvent.

To reference to the Saturated Calomel Electrode (SCE), E0 (vs. SHE) is converted to E⁰ (vs. SCE) using E⁰ (vs. SCE)=E⁰ (vs. SHE)−0.24 V. Triplet energies (in eV) of PCs were obtained by [G(³PC*)−G(¹PC), in kcal/mol]/23.06.

Based on the comparison of our large experimental and computational data set, the choice of CPCM solvation model is justified as the computed reduction potential closely approximates the experimental values. For example, the computed ground state oxidation potentials between the ²PC^{●+}/¹PC redox couple is typically within ˜0.2 to 0.3 V from the experimental values.

(d) Excited State Calculation

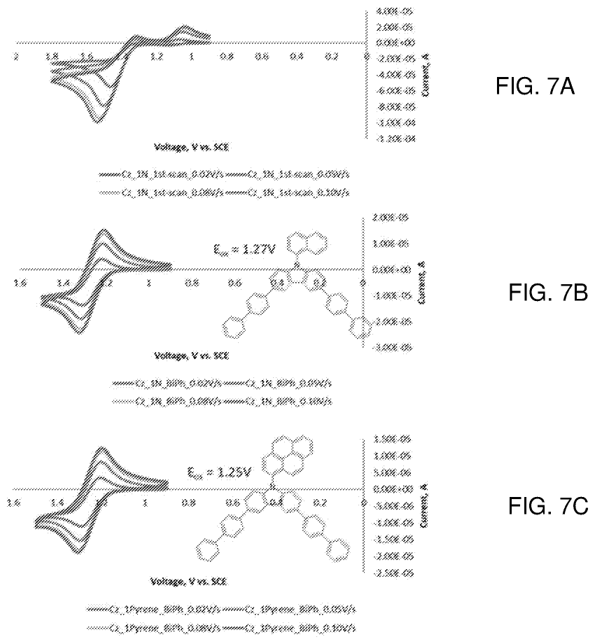

Using optimized ground state geometries, single point time dependent density functional theory (TD-DFT) calculations were performed using the rCAM-B3LYP/6-31+G(d,p)/CPCM-DMA level of theory. (Yanai, T.; Tew, D. P.; Handy, N. C. Chemical Physics Letters 2004, 393, 51-57) rCAM-B3LYP was chosen because it gave better λₘₐₓ predictions that are closer to experimental values in comparison to rωB97xd level of theory. TD-DFT calculations (with our chosen CAM-B3LYP method) corroborate experimental observations that UV-vis absorption becomes increasingly red-shifted with higher molar absorptivity as the aryl conjugation at the core position is increased. FIGS. 7A-7C shows the Electrochemical Stability and Experimental Oxidation Potentials for some characteristic carbazole PCs. FIG. 3 shows the UV-vis data for acridine PCs 1-3.

### Summary of Acridine PC Properties

TABLE 3
Summary of Photophysical and Electrochemical data for Acridine PCs 1-7.

| | | | | | | | E_{1/2} | E⁰ₒₓ | E⁰*_{S1, exp.} | E⁰*_{T1, calc.} |
|---|---|---|---|---|---|---|---|---|---|---|
| | λ_{max, abs} | ε | | λ_{max, em} | E_{S1, exp} | E_{T1, Calc} | (²PC^{•+}/¹PC) | (²PC^{•+}/¹PC) | (²PC^{•+}/¹PC*) | (²PC^{•+}/¹PC*) |
| PC | (nm)^{a} | (M⁻¹cm⁻¹) | f | (nm)^{b} | (eV)^{c} | (eV)^{d} | (V vs. SCE)^{e} | (V vs. SCE) | (V vs. SCE)^{f} | (V vs. SCE) |
| 1 | 361 | 46,270 | 1.689 | 487 | 2.55 | 2.41 | 0.76 | 0.55 | −1.79 | −1.86 |
| 2 | 340 | 38,560 | 1.211 | 509 | 2.44 | 2.36 | 0.71 | 0.42 | −1.73 | −1.94 |
| 3 | 382 | 44,340 | 1.446 | 458 | 2.71 | 2.43 | 0.90 | 0.74 | −1.81 | −1.69 |
| 4 | 360 | 49,780 | 1.655 | 453 | 2.74 | 2.30 | 0.77 | 0.53 | −1.98 | −1.77 |
| 5 | 361 | 31,500 | 1.748 | 443 | 2.80 | 2.29 | 0.76 | 0.54 | −2.04 | −1.75 |
| 6 | 363 | 43,120 | 1.741 | 444 | 2.79 | 2.28 | 0.75 | 0.50 | −2.04 | −1.78 |
| 7 | 355 | 50,140 | 1.749 | 535 | 2.32 | 2.39 | 0.82 | 0.61 | −1.50 | −1.78 |

^{a}Absorption wavelength measured using ultraviolet-visible spectroscopy in DMF.
^{b}Emission wavelength measured using fluorescence spectroscopy in DMF.
^{c}Singlet energies were calculated using the maximum wavelength of emission.
^{d}DFT calculations performed at uM06/6-311 + Gdp/uM06/6-31 + Gdp level of theory with CPCM-described solvation in aqueous solvent.
^{e}All measurements were performed in a 3-compartment electrochemical cell with an Ag/AgNO₃ reference electrode in MeCN (0.01M) and 0.1M NBu₄PF₆ electrolyte solution. DMF was used to solvate the PCs. Platinum was used at the working and counter electrodes.
^{f}Singlet excited state reduction potentials were calculated using the singlet energies estimated from the maximum wavelength of emission and the experimentally measured E_{1/2}.

CT characteristics can be predicted through the presence of charge-separated singly occupied molecular orbitals (SOMOs) for ³PC*. Of the PCs evaluated in this study, PCs 1, 2, and 7, which possess either electron withdrawing groups or an extended π system as the N-aryl moiety, were predicted to have localization of the higher-lying SOMO onto the N-aryl substituent. PCs 4, 5, and 6 all showed a higher-lying SOMO localization onto one core substituent, while PC 3 showed a higher-lying SOMO distributed across both core substituents.

### Absolute Fluorescence Quantum Yields

Absolute fluorescence quantum yields (AFQY) of PCs 1-7 were measured using an FS5 Spectrofluorometer from Edinburg Instruments with an SC-30 Integrating Sphere accessory using a direct excitation measurement method. Measurement was made over the photocatalyst samples (S) and reference solvents (R) scattering (Rₛ and Sₛ) and emission (Rₑ and Sₑ). The equation for the calculation of AFQY using the direct excitation method is as follows:

$$AFQY = \frac{{S}_{e} - {R}_{e}}{{R}_{s} - {S}_{s}} \times 100$$

Scattering and emission spectral regions were measured separately. An O.D. filter for the scattering region with a correction of 9.67× for Rₛ and Sₛ (in the case of excitation at 365 nm) after measurement. The transmission of the O.D. filter was measured at O.D. 0.103 using a Cary 5000 with diffuse reflectance accessory. All samples were prepared inside a nitrogen-filled glovebox at concentrations of 0.1 mM in degassed spectrochemical grade DMAc. Quartz cuvettes with white Teflon caps and a path length of 1 cm were used. The AFQY value was calculated using the Fluoracle software using the equation described above.

To represent reaction conditions, AFQY values were measured at 365 nm. PCs 3, 4, 5, and 6 showed some emission at 365 nm and so all PCs were also measured at 325 nm, with PC 2 measured with excitation at 305 nm due to strong absorption at 325 nm.

CT character can be observed through a large Stokes shift and visualized through solvatochromism, where the polar ¹PC* is progressively stabilized by increasing solvent polarity, resulting in lower-energy emission and a corresponding red-shift in λ_{max,em}. Evaluation of CT from PC* can estimate the CT character of ³PC*, as CT singlet and triplet excited states are expected to be energetically degenerate, with low ΔE_{ST}. A high fluorescence quantum yield (Φ_{f}) can indicate a lack of CT, as CT states have been shown to minimize fluorescence and increase triplet yields. Consistent with previous observations in N,N-diaryl dihydrophenazine and N-aryl phenoxazine studies, for dimethyl-dihydroacridines subtle N-aryl substitution differences were found to be significant in influencing the nature of experimentally-observed CT character. Of these candidates, PCs 1, 2, and 7 displayed the largest degree of CT through the largest measured Stokes shifts (ranging from 126 to 180 nm) paired with low Φ_{f} (0.1% to 8.7%), and the most dramatic solvatochromism spanning blue to yellow wavelengths of emission. By the same analysis, PCs 4 and 5 displayed a moderate degree of CT, while PCs 3 and 6 displayed the least amount of CT character. Notably, Φ_{f} values ranging from 0.1% (PC 2) to 83% (PC 3) can be obtained by modulating core-substitution.

TABLE 4
Results of measurement of fluorescence quantum yields of
PCs 1-7 with excitation at 365 nm and 325 nm.

| PC | Φ_{f, 365 nm} | Φ_{f, 325 nm} |
|---|---|---|
| 1 | 0.1 | 0.3 |
| 2 | 0.7 | 0.1 |
| 3 | 94 | 83 |
| 4 | 5.7 | 3.8 |
| 5 | 38 | 27 |
| 6 | 98 | 70 |
| 7 | 6.4 | 8.2 |

Due to strong PC absorption at 325 nm, 365 nm was used for excitation wavelength.

### Cyclic Voltammetry

(a) General Procedure for Initiators

Cyclic voltammetry of ATRP initiators was performed in a 3-compartment electrochemical cell with Ag/AgNO₃ (0.01 M) in acetonitrile as the reference electrode, TBAPF6 in DMF (0.1 M) as the electrolyte solution, and platinum for the working and counter electrodes. All solutions were prepared on the benchtop, then sparged with nitrogen for 15 minutes before analysis. 1 mM solutions of analyte were used. Scans were performed at 100 mV/s with 2 cycles each. Ep/2 values were determined from the average of the onset of reduction and peak potential on the first cycle.

(b) General Procedure for PCs

Cyclic voltammetry of PCs 1-7 and their precursor compounds were performed in a 3-compartment electrochemical cell with Ag/AgNO₃ (0.01 M) in acetonitrile as the reference electrode, TBAPF₆ in DMF (0.1 M) as the electrolyte solution, and platinum for the working and counter electrodes. All solutions were prepared on the benchtop, then sparged with nitrogen for 15 minutes before analysis. For the non-reversible PC precursors, scans were conducted at 20 mV/s and 100 mV/s. Analysis of PCs 1-7 was conducted at 20 mV/s, 50 mV/s, 80 mV/s, and 100 mV/s with 5 cycles each.

Evaluation of excited-state redox potentials of these PCs was performed by DFT calculations to predict E⁰*_{T1,calc.} (²PC^{●+}/³PC*) values (Table 1). Experimentally, E⁰*_{S1,exp.} (²PC^{●+}/¹PC*) was determined by a modified Rehm-Weller equation: E⁰*_{S1,exp.}=E_{1/2}−E_{S1,exp.}, where E_{S1,exp.} was measured from the maximum wavelength of steady-state emission at room temperature. Experimental triplet energies (E_{T1,exp.}) were measured from PC phosphorescence at 77 K with a 1 ms gate-delay using time-resolved spectroscopy. E_{S1,exp.} was also evaluated at 77 K with no gate delay, finding significant shifts in emission profiles. Interestingly, the PCs with the highest Stokes shifts, PCs 2 and 7 presented the lowest ΔE_{ST} values at 77 K (0.39 and 0.34 eV) Furthermore, computational E_{T1} predictions corresponded well with experimental values, with differences less than 0.07 eV.

### Example 14: Batch Photoreactor Design and General Polymerization Procedure in Batch

Batch polymerizations were performed in a 100 mL beaker wrapped in aluminum foil with a 12-inch strip of 12 V 365 nm LEDs purchased from LED Lighting Hut. Polymerizations using 380 nm and 455 nm light sources were performed with 12 V LED strips from Creative Lighting Solutions.

In a typical polymerization procedure, a scintillation vial with a small stir bar was loaded with 3.82 mg of PC 2 (6.97 μmol, 1 eq) and brought into a nitrogen-filled glovebox. Under red-light irradiation, 1 mL of N,N-dimethylacetamide (DMAc) was added to dissolve the PC, followed by 1 mL of butyl acrylate (6.97 mmol, 1000 eq.) and 13.3 μL of Diethyl 2-bromo-2-methylmalonate (DBMM) (0.70 μmol, 10 eq). The vial was then closed and placed into the photoreactor. Aliquots were taken by withdrawing 0.1 mL of reaction solution and quenching by injecting into a sealed vial with 0.8 mL of CDCl₃ containing 250 ppm BHT with air headspace. The sample was then taken out of the glovebox, where ¹H NMR analysis was performed. The sample was then dried under ambient conditions, dissolved in THE and molecular weight analysis was performed by GPC.

### Example 15: Light Source Optimization in a Batch Polymerization

Using the above general procedure, the wavelength of the LED was studied to determine the appropriate wavelength for the polymerization using PC2.

TABLE 5
Results of light source optimizations using PC 2
conducted in batch reactor.^{a}

| | Light | | | | | |
|---|---|---|---|---|---|---|
| | Source | Time | Conv. | M_{n, calc.} | Ð | I* |
| Entry | (nm) | (min.) | (%) | (kDa) | (M_{w}/Mₙ) | (%) |
| 1 | 365 | 77 | 10.6 | 10.2 | 1.53 | 96 |
| 2 | 380 | 120 | 73 | 21.4 | 9.6 | 45 |
| 3 | 455 | 120 | 86 | 31.2 | 3.79 | 37 |

^{a}Conditions are [1000]:[10]:[1] of [BA]:[DBMM]:[PC 2] with 1 mL of BA to 1 mL of DMAc in 100 mL batch reactor beaker and performed at ambient temperatures.

The results in the above table demonstrate that 365 nm provides the appropriate dispersity of the linear polymer.

### Example 16: Photocatalyst Screen for Batch Polymerization

Using the above general procedure, a photocatalyst screen was performed to determine the relative activity of the photocatalysts.

TABLE 6
Results of O-ATRP of butyl acrylate using PCs 1-7
after 60 minutes irradiation.^{a}

| | | Conv. | M_{n, calc.} | M_{n, theo.} | Ð | I* |
|---|---|---|---|---|---|---|
| Entry | PC | (%)^{b} | (kDa)^{c} | (kDa)^{d} | (M_{w}/Mₙ^{c} | (%)^{e} |
| 1 | 1 | 65 | 9.3 | 8.6 | 1.64 | 92 |
| 2 | 2 | 77 | 10.6 | 10.2 | 1.53 | 96 |
| 3 | 3 | 42 | 31.4 | 11.0 | 4.93 | 35 |
| 4 | 4 | 68 | 9.6 | 8.9 | 1.62 | 93 |
| 5 | 5 | 59 | 8.7 | 7.8 | 1.70 | 90 |
| 6 | 6 | 72 | 25.8 | 9.5 | 3.52 | 37 |
| 7 | 7 | 76 | 10.9 | 10.1 | 1.89 | 92 |

^{a}Conditions are [1000]:[10]:[1] of [BA]:[DBMM]:[PC] with 1 eq of DMAc to BA by volume. Reactions were irradiated by 365 nm LEDs in batch conditions at ambient temperatures.
^{b}Determined by ¹H NMR.
^{c}Measured using GPC.
^{d}Calculated by (Conv × [Mon]/[Init.] × Mw_{Mon})/1000.
^{e}Initiator efficiency (I*) calculated by (Theo. Mₙ/Calc. Mₙ) × 100.

From the data presented above in Table 6, PC2 provides the lowest dispersity value.

### Example 17: Solvent Screen in Batch Polymerization

A solvent screen for the batch polymerization was evaluated.

TABLE 7
Results of different solvents on the O-ATRP of butyl acrylate
using PC 2 after 60 minutes.^{a}

| | | Conv. | M_{n, calc.} | M_{n, theo}. | Ð | I* |
|---|---|---|---|---|---|---|
| Entry | Solvent | (%)^{b} | (kDa) | (kDa) | (M_{w}/Mₙ) | (%) |
| 1 | DMAc | 77 | 10.6 | 10.2 | 1.53 | 96 |
| 2 | DMF | 67 | 9.3 | 8.8 | 1.58 | 94 |
| 3 | DMSO | 71 | 9.7 | 9.4 | 2.37 | 97 |
| 4 | THF | 88 | 11.2 | 11.6 | 2.42 | 103 |
| 5 | Benzene | 92 | 24.6 | 12.1 | 4.47 | 49 |

^{a}Conditions are [1000]:[10]:[1] of [BA]:[DBMM]:[PC] with 1 eq of solvent to BA by volume. Reactions were irradiated by 365 nm LEDs in batch conditions at ambient temperatures.

From the data presented in the table above, either DMAc or DMF would be an appropriate solvent for the batch polymerization.

### Example 18: Initiator Screen for the Batch Polymerization

An initiator screen was conducted to determine the best initiator to be used in the batch polymerization.

TABLE 8
Results of differing alkyl halide initiators on the O-ATRP of BA
using PC 2 after 60 minutes.^{a}

| | | Conv. | M_{n, calc.} | M_{n, theo.} | Ð | I* |
|---|---|---|---|---|---|---|
| Entry | Initiator | (%)^{b} | (kDa) | (kDa) | (M_{w}/Mₙ) | (%) |
| 1 | DBMM | 77 | 10.6 | 10.2 | 1.53 | 96 |
| 2 | M2BP | 76 | 17.5 | 9.9 | 1.97 | 57 |
| 3 | 2BrCN | 57 | 8.8 | 7.5 | 1.66 | 85 |

^{a}Conditions are [1000]:[10]:[1] of [BA]:[RX]:[PC 2] with 1 eq DMAc relative to 1 mL BA and were irradiated by 365 nm light in batch reactor conditions with ambient temperature.

### Example 19: The Effect of Reaction Concentration

This experiment was conducted to determine the appropriate concentration for the batch polymerization.

TABLE 9
Results of changing reaction concentration on the O-ATRP of BA
using PC 2 after 2.5 hours.^{a}

| | DMAc:BA | Conv. | M_{n, calc.} | M_{n, theo.} | Ð | I* |
|---|---|---|---|---|---|---|
| Entry | (v/v) | (%)^{b} | (kDa) | (kDa) | (M_{w}/Mₙ) | (%) |
| 1 | 2:1 | 90 | 12/0 | 11/7 | 1.43 | 98 |
| 2 | 1.5:1 | 89 | 11.1 | 11.6 | 1.52 | 104 |
| 3 | 1:1 | 92 | 10.3 | 1.52 | 12.0 | 117 |
| 4 | 1:2 | 91 | 12.7 | 1.64 | 12.0 | 94 |

^{a}Conditions are [1000]:[10]:[1] of [BA]:[DBMM]:[PC 2] and were irradiated by 365 nm light in batch reactor conditions with ambient temperature.

The results show 1.5:1 of solvent to acrylate provided the dispersity while maintaining a high initiator efficiency (I*).

### Example 20: Effect of PC Loading on Batch Polymerization

This experiment was to evaluate the PC loading in the batch polymerization.

TABLE 10
Results of O-ATRP of BA after 1 hour testing the
effect of PC 2 loadings.^{a}

| | Mol % | Conv. | M_{n, calc.} | M_{n, theo.} | Ð | I* |
|---|---|---|---|---|---|---|
| Entry | PC 2 | (%)^{b} | (kDa) | (kDa) | (M_{w}/Mₙ) | (%) |
| 1 | 0.1 | 89 | 11.1 | 11.6 | 1.52 | 104 |
| 2 | 0.075 | 77 | 10.2 | 10.1 | 1.57 | 100 |
| 3 | 0.05 | 77 | 10.9 | 10.1 | 1.55 | 92 |
| 4 | 0.025 | 86 | 10.7 | 11.2 | 1.70 | 104 |
| 5 | 0.01 | 88 | 16.3 | 11.5 | 2.13 | 71 |

^{a}Conditions are [1000]:[10]:[X] of [BA]:[DBMM]:[PC 2] with 1.5 equivalents of DMAc to BA by volume. Polymerizations were irradiated by 365 nm light in batch reactor conditions with ambient temperature.

As the table above demonstrates, a concentration of 0.1% provides the best conversion, the best dispersity, and high initiator efficiency (I*).

From the above batch conditions tested, the following conditions provided the best conversion, the best dispersity, with high initiator efficiency (I*): [1000[:[10]:[1] of [BA]:[DBMM]:[PC 2] with 1.5 mL DMAc to 1 mL BA, irradiated by 365 nm LEDs at ambient temperature.

### Example 21: Flow Reactor Design and General Polymerization Procedure in Continuous Flow

Flow polymerizations were performed using a Hepatochem Photoredox Temperature Controlled reactor with a 2 mL flow attachment, also purchased directly from Hepatochem and especially configured for this photoreactor. The light source used was a 18 W 365 nm EvoluChem bulb (part no. HCK1012-01-011 from Hepatochem). The flow tubing was 1/16 in O.D. and 0.003 in I.D. with PFA as the tubing material, with inlet and outlet tubing purchased from IDEX Health and Science. All ferrules and fittings were purchased from IDEX Health and Science. The flow rate was controlled using a Pump 11 Elite Syringe Pump from Harvard Apparatus with a 50 mL stainless steel syringe fitted with chemically resistant Kalrez 0-rings.

In a polymerization experiment, a vial was loaded with 26.7 mg of Acrid-1N-OMe (0.048 μmol, 1 eq.), then brought into a nitrogen-filled glovebox. Under red light irradiation, 7 mL of DMAc and 7 mL of butyl acrylate (0.048 mol, 1000 eq.) was then added. Once all catalyst was fully dissolved, 93.3 μL of DBMM (0.488 μmol, 10 eq.) was added using a glass syringe. The reaction mixture was then transferred to a 50 mL stainless steel syringe and the first section of tubing attached. The syringe was then removed from the glovebox. Excess gas was pushed out of the syringe, then the first section of tubing was quickly connected to the reactor. The reaction was started with the initial flow rate using a syringe pump. The temperature of the reactor was controlled using a recirculator set to 22° C., which recirculated a 1/1 v/v mixture of ethylene glycol and water. The timing for the first equilibration period was set after 1 mL of initial infusion volume. For all timepoints, an equilibration period of 1.25 times the residence time was performed, followed by 0.125 times of collection time. The resulting polymer was collected directly into a vial containing 1 mL of BHT-deuterated chloroform. Conversion analysis was performed using H NMR and molecular weight analysis was performed using SEC-MALS GPC.

### Example 22: Evaluation of PCs in Continuous Flow Polymerization

This experiment is designed to determine the most appropriate PC to be used in continuous flow polymerization as described above.

TABLE 11
Results of O-ATRP of BA using PCs 1-7 in continuous flow.^{a}

| | | Res. | | | | | |
|---|---|---|---|---|---|---|---|
| | | Time | Conv. | M_{n, calc.} | M_{n, theo.} | Ð | I* |
| Entry | PC | (min.) | (%)^{b} | (kDa) ^{c} | (kDa) ^{d} | (M_{w}/Mₙ) ^{c} | (%)^{e} |
| 1 | 1 | 45 | 67 | 8.9 | 8.9 | 1.59 | 100 |
| 2 | 2 | 45 | 81 | 11.0 | 10.6 | 1.35 | 97 |
| 3 | 3 | 30 | 71 | 13.1 | 9.5 | 4.57 | 72 |
| 4 | 4 | 45 | 81 | 11.1 | 10.7 | 1.48 | 96 |
| 5 | 5 | 45 | 79 | 10.2 | 10.4 | 1.48 | 102 |
| 6 | 6 | 30 | 82 | 11.4 | 10.7 | 3.58 | 94 |
| 7 | 7 | 45 | 73 | 9.6 | 9.6 | 1.54 | 100 |

^{a}Conditions are [1000]:[10]:[1] of [BA]:[DBMM]:[PC] with 1 eq of DMAc to BA by volume. Reactions were performed in continuous flow and irradiated by 18 W 365 nm LEDs at 22° C.

As for the batch polymerization, PC2 provided the best conversion, the best dispersity, and high initiator efficiency.

### Example 23: Initiator Screen in Continuous Flow Polymerization

An initiator screen was conducted to determine the appropriate initiator to be used.

TABLE 12
Results of differing alkyl halide initiators on the O-ATRP of BA
in continuous flow using PC 2 after 45 minutes residence time.^{a}

| | | Conv. | M_{n, calc.} | M_{n, theo.} | Ð | I* |
|---|---|---|---|---|---|---|
| Entry | Initiator | (%)^{b} | (kDa) | (kDa) | (M_{w}/Mₙ) | (%) |
| 1 | DBMM | 81 | 11.0 | 10.6 | 1.35 | 97 |
| 2 | MBiB | 79 | 10.8 | 10.3 | 1.43 | 96 |
| 3 | 2BrPN | 74 | 10.8 | 9.8 | 1.34 | 91 |

^{a}Conditions are [1000]:[10]:[1] of [BA]:[RX]:[PC 2] with 1 eq DMAc relative to 1 mL BA and were irradiated by 365 nm light in continuous flow conditions at 22°.

DBMM showed the best conversion, the best dispersity, and a high initiator efficiency.

### Example 24: Concentration Effect in Continuous Flow Polymerization

The experiment was designed to optimize the concentration of the polymerization in continuous flow.

TABLE 13
Results of changing reaction concentration on the O-ATRP of BA
using PC 2 in continuous flow after 45 minutes residence time.^{a}

| | DMAc:BA | Conv. | M_{n, calc.} | M_{n, theo.} | Ð | I* |
|---|---|---|---|---|---|---|
| Entry | (v/v) | (%)^{b} | (kDa) | (kDa) | (M_{w}/Mₙ) | (%) |
| 1 | 2:1 | 87 | 13.4 | 11.9 | 1.31 | 89 |
| 2 | 1.5:1 | 80 | 10.6 | 10.6 | 1.37 | 100 |
| 4 | 1:1 | 81 | 11.0 | 10.6 | 1.35 | 97 |

^{a}Conditions are [1000]:[10]:[1] of [BA]:[DBMM]:[PC 2] and were irradiated by 365 nm light in flow conditions at 22° C.

As the above data in the table shows, a DMAc:BA concentration the best dispersity and a high initiator efficiency.

### Example 25: Catalyst Loading in Continuous Flow Polymerization

This experiment was designed to select the most appropriate PC concentration for the continuous flow polymerization.

TABLE 14
Results of O-ATRP of BA in continuous flow after 45 minutes
residence time testing the effect of PC 2 loadings.^{a}

| | Mol % | Conv. | M_{n, calc.} | M_{n, theo.} | Ð | I* |
|---|---|---|---|---|---|---|
| Entry | PC 2 | (%)^{b} | (kDa) | (kDa) | (M_{w}/Mₙ) | (%) |
| 1 | 0.1 | 80 | 10.6 | 10.6 | 1.37 | 100 |
| 2 | 0.075 | 80 | 10.1 | 10.6 | 1.43 | 105 |
| 5 | 0.05 | 81 | 10.2 | 10.6 | 1.41 | 104 |

^{a}Conditions are [1000]:[10]:[X] of [BA]:[DBMM]:[PC 2] with 1.5 equivalents of DMAc to BA by volume. Polymerizations were irradiated by 365 nm light in continuous flow reactor conditions at 22° C.

As the above data demonstrated, a 0.1 mole % concentration provides the most appropriate dispersity.

### Example 26: The Salt Additive Effect on the Continuous Flow Polymerization

This experiment shows the effect of different salt additives on the continuous flow polymerization.

TABLE 15
Results of O-ATRP of butyl acrylate using acridine
PC 2 in continuous flow testing the effect of
various salt additives on polymerization.^{a}

| | | Res. | | | | | |
|---|---|---|---|---|---|---|---|
| | | Time | Conv. | kₐₚₚ | M_{n, calc.} | Ð | I* |
| Entry | Salt | (min.) | (%) | (s⁻¹) | (kDa) | (M_{w}/Mₙ) | (%) |
| 1 | LiBr | 90 | 79 | 0.0177 | 10.0 | 1.23 | 104 |
| 2 | LiBr^{b} | 60 | 0 | — | — | — | — |
| 3 | NaBr | 90 | 81 | 0.0187 | 9.2 | 1.36 | 116 |
| 4 | KBr | 60 | 80 | 0.0278 | 9.4 | 1.41 | 112 |
| 5 | TBABr | 90 | 82 | 0.0201 | 15.0 | 1.20 | 72 |
| 6 | LiCl | 60 | 87 | 0.0354 | 27.2 | 1.98 | 42 |
| 7 | LiI | 60 | 0 | — | — | — | — |
| 9 | LiPF₆ | 60 | 92 | 0.0424 | 14.7 | 1.31 | 82 |

^{a}Conditions are [1000]:[1]:[10] of [BA]:[DBMM]:[PC 2]:[Salt] with 1.5 equivalents of DMAc to BA by volume. Polymerizations were irradiated by 18 W 365 nm light in flow reactor conditions at 22° C.
^{b}No DBMM.

As shown above, lithium bromide provided the best dispersity at a high initiator efficiency.

### Example 27: The Concentration Effect of Lithium Bromide on the Continuous Flow Polymerization

This experiment is designed to determine the appropriate lithium bromide loading on the continuous flow polymerization.

TABLE 16
Results of O-ATRP of BA using PC 2 in continuous flow testing
the effect of LiBr concentration on polymerization.^{a}

| | | Res. | | | | | |
|---|---|---|---|---|---|---|---|
| | | Time | Conv. | kₐₚₚ | M_{n, calc.} | Ð | I* |
| Entry | [LiBr]:[PC 2] | (min.) | (%) | (s⁻¹) | (kDa) | (M_{w}/Mₙ) | (%) |
| 1 | [5]:[1] | 60 | 73 | 0.0227 | 9.1 | 1.30 | 106 |
| 2 | [10]:[1] | 90 | 79 | 0.0177 | 10.0 | 1.23 | 104 |
| 3 | [20]:[1] | 90 | 60 | 0.0101 | 7.7 | 1.30 | 104 |
| 4 | [30]:[1] | 120 | 73 | 0.0111 | 9.4 | 1.20 | 103 |
| 5 | [50]:[1] | 120 | 83 | 10.1 | 10.9 | 1.23 | 108 |

^{a}Conditions are [1000]:[10]:[1]:[X] of [BA]:[DBMM]:[PC 2]:[LiBr] with 1.5 equivalents of DMAc to BA by volume. Polymerizations were irradiated by 18 W 365 nm light in flow reactor conditions at 22° C.

Even though a higher loading of lithium bromide provides a better conversion, adequate dispersity, and high initiator efficiency, an equal molar amount of LiBr and DBMM provided faster kinetics of the process.

### Example 28: Molecular Weight Control of Polymer Through Variation of Stoichiometry of Monomer and Initiator

This experiment was to determine whether varying the stoichiometry of the monomer and initiator had an effect on the molecular weight of the linear polymer in a continuous flow polymerization.

TABLE 17
Results of O-ATRP of BA using PC 2 in continuous flow targeting
different MW polymers through adjustment of stoichiometry
of monomer and initiator.^{a}

| | | Res. | | | | | |
|---|---|---|---|---|---|---|---|
| | | Time | Conv. | M_{n, calc.} | M_{n, theo.} | Ð | I* |
| Entry | [BA]:[DBMM] | (min.) | (%) | (kDa) | (kDa) | (M_{w}/Mₙ) | (%) |
| 1 | [1004]:[2.5] | 90 | 89 | 26.4 | 45.7 | 1.35 | 173 |
| 2 | [1004]:[5] | 120 | 88 | 15.2 | 22.8 | 1.32 | 150 |
| 3 | [1004]:[10] | 120 | 73 | 9.4 | 9.7 | 1.20 | 103 |
| 4 | [1004]:[20] | 180 | 80 | 7.3 | 5.4 | 1.19 | 74 |
| 5 | [1004]:[40] | 180 | 67 | 5.4 | 2.4 | 1.18 | 44 |

^{a}Conditions are [1000]:[X]:[1]:[30] of [BA]:[DBMM]:[PC 2]:[LiBr] with 1.5 equivalents of DMAc to BA by volume. Polymerizations were irradiated by 18 W 365 nm light in flow reactor conditions at 22° C.

These results show that varying the initiator stoichiometry to the acrylate monomer affects the dispersity and initiator efficiency of the process.

### Example 29: Polymerization Process with Various Acrylates and Methacrylates

This experiment was to demonstrate how different acrylates and methacrylates act in the optimized conditions.

TABLE 18
Results of O-ATRP of acrylate and methacrylate monomers using PC 2 in continuous flow.^{a}

| | | | Res. | | | | | |
|---|---|---|---|---|---|---|---|---|
| | | | Time | Conv. | M_{n, calc,} | Ð | I* | Conv. |
| Entry | Monomer | [Monomer]:[DBMM] | (min.) | (%) | (kDa) | (M_{w}/Mₙ) | (%) | (%) |
| 1 | n-BA | [1000]:[2.5] | 90 | 89 | 26.4 | 45.7 | 1.35 | 173 |
| 2 | n-BA | [1000]:[5] | 120 | 88 | 15.2 | 22.8 | 1.32 | 150 |
| 3 | n-BA | [1000]:[10] | 120 | 73 | 9.4 | 9.7 | 1.20 | 103 |
| 4 | n-BA | [1000]:[20] | 180 | 80 | 7.3 | 5.4 | 1.19 | 74 |
| 5 | n-BA | [1000]:[40] | 180 | 67 | 5.4 | 2.4 | 1.18 | 44 |
| 6^{[b]} | n-BA | [1000]:[10] | 120 | 85 | 10.3 | 11.1 | 1.24 | 108 |
| 7 | t-BA | [1000]:[10] | 120 | 92 | 13.8 | 12.1 | 1.23 | 88 |
| 8 | MA | [1000]:[10] | 90 | 92 | 10.0 | 8.1 | 1.30 | 81 |
| 9 | EA | [1000]:[10] | 90 | 76 | 7.5 | 7.8 | 1.19 | 105 |
| 10 | 2-EHA | [1000]:[10] | 90 | 90 | 16.3 | 16.8 | 1.53 | 104 |
| 11 | EGMEA | [1000]:[10] | 60 | 92 | 10.5 | 12.3 | 1.37 | 117 |
| 12 | MMA | [1000]:[10] | 600 | 72 | 8.8 | 7.4 | 1.17 | 85 |

^{a}Conditions are [1000]:[10]:[1]:[30] of [Monomer]:[DBMM]:[PC 2]:[LiBr] with 1.5 equivalents of DMAc to BA by volume. Polymerizations were irradiated by 18 W 365 nm light in flow reactor conditions at 22° C.
^{[b]}Reaction components sparged with air for 30 minutes before polymerization.

In batch conditions, no monomer conversion was observed using PC 2 under ambient atmosphere. To further test the oxygen-tolerance of dimethyl-dihydroacridines, the 0-ATRP of BA was performed in optimized flow conditions using reagents and solvents previously exposed proceeded efficiently with no observed induction period, with Ð<1.24 and I* close to 100% (Table 18, entry 6).

All acrylates and methacrylates above reacted in the process where lower esters provided the best dispersity.

### Example 30: Chain Extension Experiments

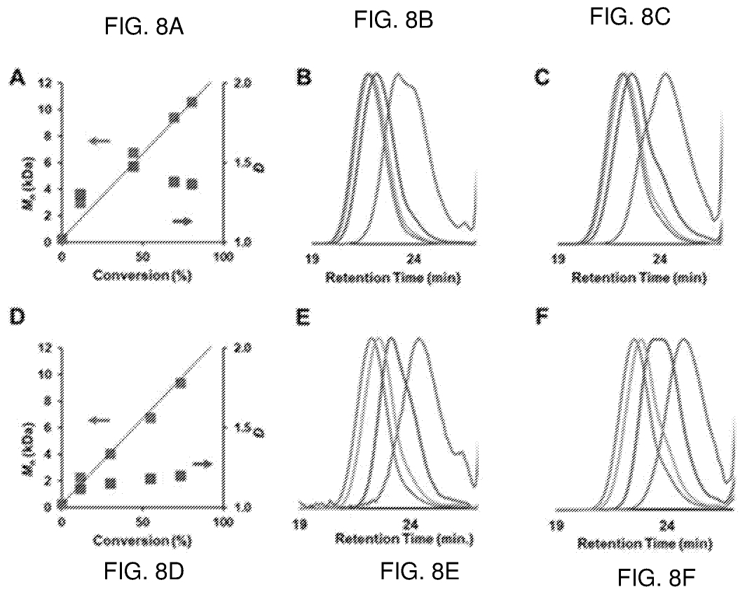

To validate the chain-end group fidelity of the system and demonstrate the ability of this system to produce complex polymeric materials, chain-extensions were performed in continuous-flow of an isolated p(n-BA) macroinitiator (Mₙ=4.6 kDa, Ð=1.26) with ethyl acrylate (EA) to produce a block-copolymer p(n-BA)-b-p(EA) with Ð=1.16 (12 kDa). This polymer was then again reintroduced as a macroinitiator and further extended with tert-butyl acrylate to produce a well-defined triblock copolymer (Mₙ=20 kDa, Ð=1.44) (FIGS. 8A-8F).

The following triblock copolymers was conducted as shown in the below chemical scheme using procedures above:

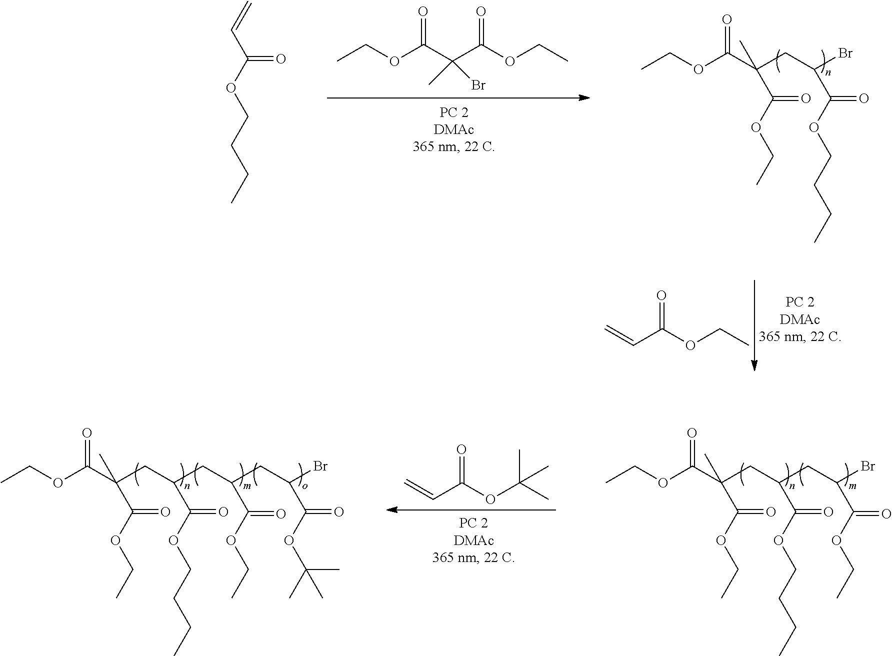

(a) Macroinitiator Synthesis:

A p(BA) macroinitiator was synthesized using optimized O-ATRP conditions. Inside a nitrogen-filled glovebox, 107.4 mg (1.24 mmol, 30 eq.) of LiBr was dissolved in 9 mL of DMAc. Then, the solution was transferred to a vial with 22.8 mg (0.041 mmol, 1 eq.) of PC 2. 6 mL of BA (41.7 mmol, 1000 eq.) was added, followed by 79.6 μL DBMM (0.41 mmol, 10 eq.). The solution was then transferred to a stainless-steel syringe, which was then fitted with the first section of PFA tubing. The syringe was taken out of the glovebox and quickly connected to the flow reactor, which was set to a 30 minute residence time. After equilibration for 1.25× residence time, the product was collected for 2 hours. Monomer conversion was measured at 27.5%. After precipitation in cold methanol/water, the polymer was dried to constant weight under vacuum at 50° C. to give 1.32 g with Mₙ=4.57 kDa, Ð=1.26, and I*=83%.

(b) Block Copolymer Synthesis:

A p(BA)-c-p(EA) block copolymer was synthesized using a [200]:[1]:[0.1]:[3] ratio of [EA]:[pBA]:[PC 2]:[LiBr]. 22.6 mg LiBr (0.262 mmol) was dissolved in 3.72 mL of DMAc. The solution was then transferred to a vial with 4.79 mg PC 2 (0.0087 mmol), which was then transferred to a vial with 400 mg of p(BA) macroinitiator (0.087 mmol). After the polymer was dissolved, 1.86 mL of EA (17.5 mmol) was added. The solution was then transferred to a stainless-steel syringe, which was then fitted with the first section of PFA tubing. The syringe was taken out of the glovebox and quickly connected to the flow reactor, which was set to a 60 minute residence time. After equilibration for 1.25× residence time, the product was collected for 1 hour. After precipitation in cold methanol/water, the polymer was dried to constant weight under vacuum at 50° C. to give 0.45 g with Mₙ=12.6 kDa, Ð=1.16.

(c) Triblock Copolymer Synthesis:

A p(BA)-c-p(EA)-c-(t-BA) triblock copolymer was synthesized using a [200]:[1]:[0.1]:[3] ratio of [t-BA]:[block copolymer]:[PC 2]:[LiBr]. 8.2 mg LiBr (0.095 mmol) was dissolved in 2.80 mL of DMAc. The solution was then transferred to a vial with 1.7 mg PC 2 (3.18 μmol), which was then transferred to a vial with 400 mg of p(BA)-c-p(EA) macroinitiator (0.032 mmol). After the polymer was dissolved, 0.93 mL of t-BA (6.4 mmol) was added. The solution was then transferred to a stainless-steel syringe, which was then fitted with the first section of PFA tubing. The syringe was taken out of the glovebox and quickly connected to the flow reactor, which was set to a 60 minute residence time. After equilibration for 1.25× residence time, the product was collected for 30 minutes. After precipitation in cold methanol/water, the polymer was dried to constant weight under vacuum at 50° C. to give 0.2 g with Mₙ=20 kDa, Ð=1.44. FIGS. 8A-8F8 illustrate the triblock copolymer and the associated data. Plots of MW vs conversion (blue) and Ð vs. conversion (red) for O-ATRP of butyl acrylate without LiBr (A) and with LiBr (D). Corresponding GPC MALS traces are shown in B and E, while corresponding GPC RI traces are shown in C and F.

### Example 31: C—N Cross Coupling Using Dual Carbazole Photoredox (Cz-1Naph-Biphen) and Nickel Catalysis

C—N cross coupling reactions were performed according to the following procedure. Under a nitrogen atmosphere in a glovebox, either DABCO (80.8 mg, 0.72 mmol, 1.8 eq, Conditions A) or K₂CO₃ (99.5 mg, 0.72 mmol, 1.8 eq, Conditions B) was added to a 1.0 dram glass vial charged with a stir bar. For Conditions A, no ligand was added. For Conditions B, 4,4′-di-tert-butyl-2,2′-bipyridine (5.4 mg, 0.02 mmol, 0.05 eq) was added to the vial. 4-bromobenzotrifluoride (56 μL, 1.0 eq., 0.4 mmol) was then added via micropipette. A DMAc solution containing dissolved NiBr₂.glyme (0.200 mL, 0.02 mmol, 0.05 eq) was then added to the vial. A solution of photocatalyst (0.0016 mmol, 0.004 eq.) dissolved in DMAc (0.500 mL) was added via micropipette. Finally, morpholine (51.8 μL, 0.60 mmol, 1.5 eq) was added to the vial. The glass vial was then capped using a screw cap and sealed with Parafilm®. The vial was removed from the glovebox and subjected to LED irradiation in the light beaker setups described above, with white LEDs for all photocatalysts except catalyst e), Cz-1naph-biphen, for which UV LEDs were used. After 24 hours, the reaction was stopped by turning off the reactor and a 15 μL aliquot was removed for ¹⁹F NMR. The catalysts used were PhenO-1Naph-Biphen (catalyst a)=3,7-di([1,1′-biphenyl]-4-yl)-10-(naphthalen-1-yl)-10H-phenoxazine, PhenO-Pyrene (10-(pyren-1-yl)-10H-phenoxazine), or Cz-1Naph-Biphen (catalyst e)=3,6-di([1,1′-biphenyl]-4-yl)-9-(naphthalen-1-yl)-9H-carbazole.

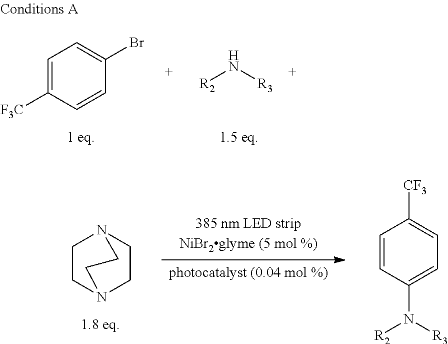

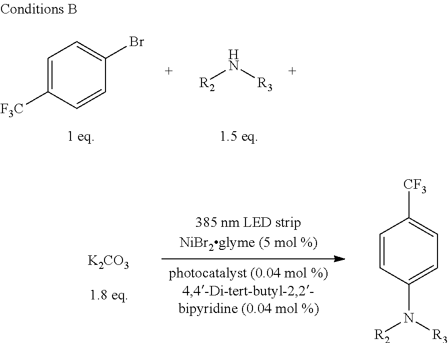

Results from the C—N cross-coupling comparing Cz-1Naph-Biphen with phenoxazine catalysts. Conversions of the processes are shown below in Table 19 which were determined by ¹⁹F NMR.

TABLE 19

| | Cz-1Naph- | PhenO- | PhenO- |
|---|---|---|---|
| Reaction | Biphen | Pyrene | 1Naph-Biphen |
| C-N, conditions A | 48.3% | 34.7% | 62.9% |
| C-N, conditions B | 22.1% | 3.4% | Not run |

As the data indicates in the above table, both carbazole and phenoxazine are suitable catalysts for C—N cross coupling.

All publications and patent applications cited in this specification are herein incorporated by reference as if each individual publication or patent application were specifically, and individually, indicated to be incorporated by reference.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A compound comprising Formula (IV) or a salt thereof: 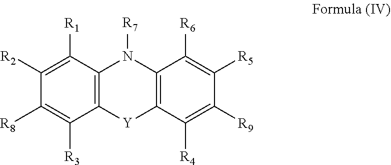
wherein:
R₁, R₂, R₃, R₄, R₅, and R₆ are independently selected from a group consisting of hydrogen, C₁-C₈ substituted or unsubstituted alkyl optionally substituted with at least one heteroatom; C₆-C₂₀ substituted or unsubstituted aryl optionally substituted with at least one heteroatom;
R₇ is selected group a group consisting of C₁-C₈ substituted or unsubstituted alkyl optionally substituted with at least one heteroatom; C₆-C₂₀ substituted or unsubstituted aryl optionally substituted with at least one heteroatom;
R₈ and R₉ are independently selected from a group consisting of C₆-C₂₀ substituted or unsubstituted aryl optionally substituted with at least one heteroatom;
Y is O, CR₁₀R₁₁, or absent; and
R₁₀ and R₁₁ are independently selected from a group consisting of hydrogen, C₁-C₈ substituted or unsubstituted alkyl optionally substituted with at least one heteroatom and C₆-C₁₄ substituted or unsubstituted aryl optionally substituted with at least one heteroatom.

2. The compound of claim 1, wherein R₁, R₂, R₃, R₄, R₅, and R₆ are independently selected from a group consisting of hydrogen, C₁-C₆ substituted or unsubstituted alkyl optionally substituted with at least one heteroatom; C₆-C₁₄ substituted or unsubstituted aryl optionally substituted with at least one heteroatom;
R₇ is selected from a group consisting of C₁-C₆ substituted or unsubstituted alkyl optionally substituted with at least one heteroatom; C₆-C₁₄ substituted or unsubstituted aryl optionally substituted with at least one heteroatom;
R₈ and R₉ are independently selected from a group consisting of C₆-C₂₀ substituted or unsubstituted aryl optionally substituted with at least one heteroatom;
Y is O, CR₁₀R₁₁, or absent; and
R₁₀ and R₁₁ are independently selected from a group consisting of hydrogen, C₁-C₆ substituted or unsubstituted alkyl optionally substituted with at least one heteroatom; and C₆-C₁₄ substituted or unsubstituted aryl optionally substituted with at least one heteroatom.

3. The compound of claim 1, wherein R₁, R₂, R₃, R₄, R₅, and R₆ are independently selected from a group consisting of hydrogen, C₁-C₄ substituted or unsubstituted alkyl optionally substituted with at least one heteroatom; C₆-C₁₄ substituted or unsubstituted aryl optionally substituted with at least one hetero atom;
R₇ is selected from a group consisting of C₁-C₄ substituted or unsubstituted alkyl optionally substituted with at least one heteroatom; C₆-C₁₄ substituted or unsubstituted aryl optionally substituted with at least one heteroatom;
R₈ and R₉ are independently selected from a group consisting of C₆-C₂₀ substituted or unsubstituted aryl optionally substituted with at least one heteroatom;
Y is O, CR₁₀R₁₁, or absent; and
R₁₀ and R₁₁ are independently selected from a group consisting of hydrogen, C₁-C₆ substituted or unsubstituted alkyl optionally substituted with at least one heteroatom; C₆-C₁₂ substituted or unsubstituted aryl optionally substituted with at least one hetero atom.

4. The compound of claim 1, wherein; R₁, R₂, R₃, R₄, R₅, and R₆ are hydrogen, R₇ is selected from a group consisting of methyl, ethyl, phenyl, 1-naphthyl, 2-naphthyl, phenyl, 4-methoxyphenyl, or 4-cyanophenyl; R₈ and R₉ are independently selected from a group consisting of 4, 4′-biphenyl, 4-methoxyphenyl, or 4-cyanophenyl; Y is O, CR₁₀R₁₁, or absent; and R₁₀ and R₁₁ are methyl.

5. The compound of claim 1, wherein the compound exhibits a molar absorptivity from about 25,000 M⁻¹cm⁻¹ to about 70,000 M⁻¹cm⁻¹.

6. The compound of claim 1, wherein the compound exhibits E⁰*(PC^{●+}/³PC*) potentials ranging from about −2.30 V vs SCE (standard calomel electrode) to about −1.00 V vs SCE.

7. The compound of claim 1, wherein the compound exhibits E_{1/2}(PC^{●+}/PC) from about 0.30 to about 1.50 versus a SCE.

8. A method for preparing a compound comprising Formula (IV): 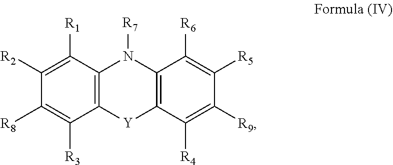
the method comprising:
(a) contacting a compound comprising Formula (I): 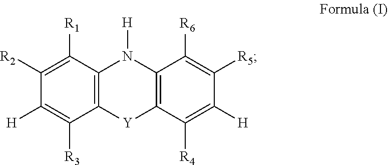
with an aromatic halide in the presence of a catalyst to form a compound comprising Formula (II): 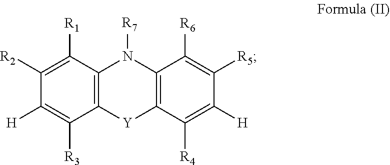
(b) contacting the compound comprising Formula (II) with a halogenating agent to form a compound comprising Formula (III): 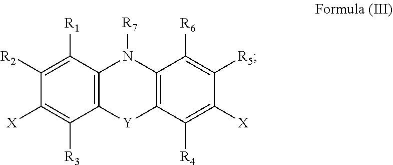
and
(c) contacting the compound comprising Formula (III) with an aryl boronic acid in the presence of a catalyst to form the compound comprising Formula (IV);
wherein:
R₁, R₂, R₃, R₄, R₅, and R₆ are independently selected from a group consisting of hydrogen, C₁-C₈ substituted or unsubstituted alkyl optionally substituted with at least one heteroatom; C₆-C₂₀ substituted or unsubstituted aryl optionally substituted with at least one hetero atom;
R₇ is selected group a group consisting of C₁-C₈ substituted or unsubstituted alkyl optionally substituted with at least one heteroatom; C₆-C₂₀ substituted or unsubstituted aryl optionally substituted with at least one hetero atom;
R₈ and R₉ are independently selected from a group consisting of C₆-C₂₀ substituted or unsubstituted aryl optionally substituted with at least one heteroatom;
Y is O, CR₁₀R₁₁, or absent;
X is Cl, Br, or I; and
R₁₀ and R₁₁ are independently selected from a group consisting of hydrogen, C₁-C₈ substituted or unsubstituted alkyl optionally substituted with at least one heteroatom and C₆-C₁₄ substituted or unsubstituted aryl optionally substituted with at least one heteroatom.

9. The method of claim 8, wherein R₁, R₂, R₃, R₄, R₅, and R₆ are independently selected from a group consisting of hydrogen, C₁-C₆ substituted or unsubstituted alkyl optionally substituted with at least one heteroatom; C₆-C₁₄ substituted or unsubstituted aryl optionally substituted with at least one hetero atom;
R₇ is selected from a group consisting of C₁-C₆ substituted or unsubstituted alkyl optionally substituted with at least one heteroatom; C₆-C₁₄ substituted or unsubstituted aryl optionally substituted with at least one heteroatom;
R₈ and R₉ are independently selected from a group consisting of C₆-C₂₀ substituted or unsubstituted aryl optionally substituted with at least one heteroatom;
Y is O, CR₁₀R₁₁, or absent; and
R₁₀ and R₁₁ are independently selected from a group consisting of hydrogen, C₁-C₆ substituted or unsubstituted alkyl optionally substituted with at least one heteroatom; C₆-C₁₂ substituted or unsubstituted aryl optionally substituted with at least one hetero atom.

10. The method of claim 8, wherein R₁, R₂, R₃, R₄, R₅, and R₆ are independently selected from a group consisting of hydrogen, C₁-C₄ substituted or unsubstituted alkyl optionally substituted with at least one heteroatom; C₆-C₁₄ substituted or unsubstituted aryl optionally substituted with at least one hetero atom;
R₇ is selected from a group consisting of C₁-C₄ substituted or unsubstituted alkyl optionally substituted with at least one heteroatom, C₆-C₁₄ substituted or unsubstituted aryl optionally substituted with at least one heteroatom;
R₈ and R₉ are independently selected from a group consisting of C₆-C₂₀ substituted or unsubstituted aryl optionally substituted with at least one heteroatom;
Y is O, CR₁₀R₁₁, or absent; and
R₁₀ and R₁₁ are independently selected from a group consisting of hydrogen, C₁-C₆ substituted or unsubstituted alkyl optionally substituted with at least one heteroatom; C₆-C₁₂ substituted or unsubstituted aryl optionally substituted with at least one heteroatom.

11. The method of claim 8, wherein R₁, R₂, R₃, R₄, R₅, and R₆ are hydrogen, R₇ is selected from a group consisting of methyl, ethyl, phenyl, 1-naphthyl, 2-naphthyl, phenyl, 4-methoxyphenyl, or 4-cyanophenyl; R₈ and R₉ are independently selected from a group consisting of 4, 4′-biphenyl, 4-methoxyphenyl, or 4-cyanophenyl; Y is O, CR₁₀R₁₁, or absent; and R₁₀ and R₁₁ are methyl.

12. The method of claim 8, wherein the mole ratio of the compound comprising Formula (I) with the aromatic halide in step (a) ranges from about 1.0:1.0 to about 1.0:2.0.

13. The method of claim 8, wherein the mole ratio of the compound comprising Formula (I) with the catalyst in step (a) ranges from about 1.0:0.001 to about 1.0:0.05.

14. The method of claim 8, wherein step (a) further comprises at least one ligand.

15. The method of claim 15, wherein the mole ratio of the catalyst to the ligand ranges in step (a) from about 1.0:0.5 to about 1.0:5.0.

16. The method of claim 8, wherein step (a) further comprises at least one base.

17. The method of claim 8, wherein the mole ratio of the compound comprising Formula (II) to the halogenating agent in step (b) ranges from about 1.0:2.0 to about 1.0:5.0.

18. The method of claim 8, wherein the mole ratio of the compound comprising Formula (III) to the aryl boronic acid in step (c) may ranges from about 1.0:2.0 to about 1.0:10.0.

19. The method of claim 8, wherein the mole ratio of the compound comprising Formula (III) to the catalyst in step (c) ranges from about 1.0:0.01 to about 1.0:0.2.

20. The method of claim 8, wherein step (c) further comprises at least one base.

21. A method for preparing a non-statistical, linear polymer, the method comprising:
(a) generating a reaction mixture comprising contacting monomer A, the compound of claim 1, an initiator (In), a salt additive, and a solvent;
(b) irradiating of the reaction mixture with UV light to generate the linear polymer In-Aₙ-X;
(c) isolating at least a portion of the linear polymer In-Aₙ-X;
(d) generating a second mixture comprising the linear polymer In-Aₙ-X from step (c), monomer B; a salt additive; and a solvent;
(e) irradiating of the second reaction mixture with UV light to form a linear polymer In-Aₙ-Bₘ—X; and
(f) isolating at least a portion of the linear polymer In-Aₙ-Bₘ—X;
wherein the non-statistical, linear polymer has a high propagation constant; X=Cl, Br, or I; and n and m are integers from 1 to 10,000.

22. The method of claim 21, wherein the method further comprises preparing a third reaction mixture comprising the linear polymer In-Aₙ-Bₘ—X, monomer C, a salt additive; irradiation of the third reaction mixture with UV light to form the linear polymer In-Aₘ-Bₙ—Cₒ—X; and isolating at least a portion of the linear polymer In-Aₙ-Bₘ—Cₒ—X.

23. The method of claim 21, wherein monomers A, B, and C may be the same or different.

24. The method of claim 21, wherein the monomers A, B, and C are independently selected from a group consisting of an acrylate ester, an acrylic acid, acrylonitrile, a methacrylate ester, methacrylic acid, or methacrylonitrile.

25. The method of claim 21, wherein the UV light source emits light from about 350 nm to about 400 nm.

26. The method of claim 21, wherein the equivalent ratio of the monomer A, B, or C to the compound of claim 1 ranges from about 1:1 to about 10,000:1.

27. The method of claim 21, wherein the equivalent ratio of the initiator to the compound of claim 1 ranges from about 1.0:1.0 to about 50.0:1.0.

28. The method of claim 21, wherein the equivalent ratio of the salt additive to the compound of claim 1 ranges from about 1.0:1.0 to about 50.0:1.0

29. The method of claim 21, wherein the volume to volume ratio of the solvent to the monomer A, B, or C ranges from about 0.1:1.0 to about 10.0:1.0.

30. The method of claim 21, wherein the process is conducted at a temperature from about 0° C. to about 50° C.

31. The method of claim 21, wherein the linear polymer has a dispersity (D) less than or equal to 1.20.

32. A dual catalytic method for forming an aryl carbon-nitrogen bond, the method comprising:
contacting an aryl halide with an amine in the presence of a dual catalytic solution comprising a Ni(II) salt catalyst, a compound of claim 1, and an optional base, thereby forming a reaction mixture; and
exposing the reaction mixture to light under reactions conditions sufficient to form the aryl carbon-nitrogen bond.

33. The method of claim 32, wherein the reactions conditions comprise holding the reaction mixture at between about room temperature and about 80° C. for between about 1 hour and about 20 hours such that at least about 50% reaction yield is obtained.
